(19) United States
(12) Patent Application Publication
(10) Pub. No.: US 2026/0102403 A1
(43) Pub. Date: Apr. 16, 2026

(54) ORAL FORMULATIONS OF THR-BETA AGONIST

(71) Applicant: ALIGOS THERAPEUTICS, INC., South San Francisco, CA (US)

(72) Inventors: Felix ShangChung Lai, Fremont, CA (US); Meenakshi Venkatraman, Fremont, CA (US); Fanny Nadine Quemeneur, Ploermel (FR); Annabel Eliane Francine Igonin, Ploermel (FR); Noorullah Naqvi Mohammed, Brisbane, CA (US)

(73) Assignee: ALIGOS THERAPEUTICS, INC., South San Francisco, CA (US)

(21) Appl. No.: 19/354,523

(22) Filed: Oct. 9, 2025

Related U.S. Application Data
(60) Provisional application No. 63/706,346, filed on Oct. 11, 2024.

(51) Int. Cl. A61K 31/53; A61K 9/00; A61K 9/48; A61K 45/06

(52) U.S. Cl. CPC A61K 31/53; A61K 9/0053; A61K 9/4825; A61K 9/4858; A61K 9/4866; A61K 45/06

(57) ABSTRACT

Disclosed herein are pharmaceutical compositions comprising a compound of Formula I:

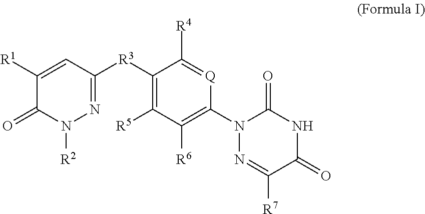

or a stereoisomer or a tautomer thereof, or a pharmaceutically acceptable salt thereof, and one or more surfactants, and methods of treating disease by administering or contacting a subject with one or more of the above pharmaceutical compositions.

### CROSS-REFERENCE TO RELATED APPLICATIONS

This application claims the benefit of priority to U.S. Provisional Patent Application Ser. No. 63/706,346, filed on Oct. 11, 2024, the entire disclosure of which is hereby incorporated by reference herein.

### FIELD OF THE DISCLOSURE

The present disclosure is in the field of pharmaceutical compositions and preparations and method of their use in the treatment of disease. In particular, the present disclosure is in the field of THR-β modulators and their use.

### BACKGROUND OF THE DISCLOSURE

In parallel with the global increase in obesity, metabolic dysfunction-associated steatotic liver disease (MASLD, formerly known as nonalcoholic fatty liver disease (NAFLD); MASLD and NAFLD are used interchangeably) is becoming the leading cause of chronic liver disease and liver transplantation worldwide [1,2]. MASLD is believed to affect 30% of the adult population and 70-80% of individuals who are obese and diabetic. MASLD is defined as excess liver fat accumulation greater than 5% induced by causes other than alcohol intake. MASLD progresses to liver inflammation (metabolic dysfunction-associated steatohepatitis (MASH), formerly known as nonalcoholic steatohepatitis, NASH; MASH and NASH are used interchangeably) and fibrosis in a variable proportion of individuals, ultimately leading to liver failure and hepatocellular carcinoma (HCC) in susceptible individuals [3].

In the United States alone, MASH is the third most common indication for liver transplantation and is on a trajectory to become the most common [4]. The most important medical need in patients with MASLD and MASH is an effective treatment to halt the progression and possibly reverse fibrosis, which is the main predictor of liver disease evolution [5,6].

Thyroid hormone (TH) is essential for normal development, growth and metabolism of all vertebrates. Its effects are mediated principally through triiodothyronine (T3), which acts as a ligand for the TH receptors (TRs, or THRs) β1, β2 and α1 [7]. In the absence of ligand, TR first binds as a heterodimer or homodimer on TH response elements (TRE) located in the promoter regions of target genes, where it interacts with corepressors. Upon ligand binding, the TR homodimers are dissociated in favor of heterodimer formation with the retinoid-X receptor (RXR), resulting in release of the corepressors and recruitment of coactivators. This new complex attracts a large number of proteins which engage the RNA polymerase II in the transcription of the targeted genes.

Two different genetic loci, denoted THRA and THRB, are responsible for encoding multiple interrelated TR isoforms that have distinct tissue distributions and biological functions. The two major isoforms with the broadest level of tissue expression are TRα1 and TRβ1 [8]. While TRα1 is expressed first during fetal development and is widely expressed in adult tissues, TRβ1 appears later in development and displays highest expression in the adult liver, kidney, and lung [9]. TRα1 is a key regulator of cardiac output, whereas TRβ1 helps in the control of metabolism in the liver. Importantly, the natural thyroid hormone T3 activates both TRα1 and TRβ1 without any significant selectivity.

Design of thyromimetic small molecule agents led to the identification of TR (or THR) agonists with varying levels of TRβ selectivity despite high structural similarity between the ligand-binding domains for TRβ and TRα. TRβ selectivity achieved by some of these compounds resulted in an improved therapeutic index for lipid lowering relative to cardiac effects such as heart rate, cardiac hypertrophy, and contractility [10-12].

Another strategy to avoid activation of TRα in cardiac tissue is to design prodrugs of phosphonate-containing TR agonists that are specifically converted to the active agonist in the liver but remain stable as an inactive prodrug in blood and extrahepatic tissues, including the heart [13]. TRα and TRβ agonists are also used in indications other than liver-related disorders, as has been known in the art. For example, TRβ selective agonists may be useful in the treatment of X-linked adrenoleukodystrophy [14, 15].

### SUMMARY

Provided herein, in one aspect, are pharmaceutical compositions comprising a compound of Formula I:

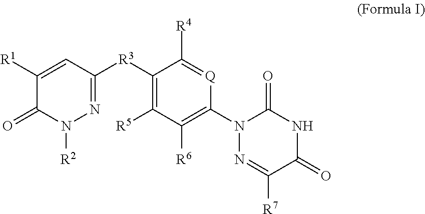

or a stereoisomer or a tautomer thereof, or a pharmaceutically acceptable salt thereof, and one or more surfactants;
wherein
R¹ is C₁-C₆ alkyl; C₂-C₁₀ alkenyl; a non-aromatic C₃-C₁₂ carbocyclic ring; a C₆-C₁₀ aryl group; a 3- to 6-membered heterocycloalkyl ring containing one to four ring heteroatoms independently selected from oxygen, sulfur, and nitrogen; a five- to ten-membered heteroaryl ring containing one to four ring heteroatoms independently selected from oxygen, sulfur, and nitrogen; a (carbocyclic)alkyl group; an aralkyl group; or a (heterocycloalkyl)alkyl group; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen;
R² is hydrogen or an optionally substituted C₁-C₆ alkyl;
R³ is independently —(C(R^{a})₂)_{z}—; oxygen; sulfur; or —NR^{a}—; wherein:
each R^{a} is independently a hydrogen or C₁-C₆ alkyl; and
z is 0, 1, 2, 3, 4 or 5;
each of R⁴ and R⁵ is independently selected from halogen, —CN, optionally substituted C₁-C₆ alkyl, optionally substituted C₁-C₆ alkoxy, optionally substituted C₂-C₆ alkenyl, or cyclopropyl;
R⁶ is selected from hydrogen, optionally substituted C₁-C₆ alkyl, optionally substituted C₁-C₆ alkoxy, cyano, or halogen;
optionally R⁵ and R⁶ taken together, along with the carbon atoms to which they are attached, form a 4-, 5- or 6-membered non-aromatic carbocyclic, heterocycloalkyl, aryl, or heteroaryl ring;
Q is nitrogen or —CR^{c}—, wherein R^{c} is hydrogen, halogen, or C₁-C₆ alkyl;
R⁷ is —(C(R^{d})₂)ₙ—N(R^{d})₂; wherein
each R^{d} is independently hydrogen or optionally substituted C₁-C₆ alkyl; and
each n is independently selected from 0 or 1; and
   wherein the composition is formulated for oral administration.

In some embodiments, the compound is present in the composition in an amount of about 0.025 wt. % to about 0.45 wt. %. In some embodiments, the one or more surfactants comprise a high hydrophilic-lipophilic balance (HLB) surfactant. In some embodiments, the high HLB surfactant is selected from the group consisting of polysorbate 80, PEG 35 castor oil, PEG 40 hydrogenated castor oil (also referred to as macrogolglycerol hydroxystearate), PEG 32 lauroyl glycerides, PEG 32 stearate, PEG 32 stearoyl glycerides, D alpha tocopherol PEG 1000 succinate, and any combination of two or more thereof. In some embodiments, the high HLB surfactant is castor oil. In some embodiments, the high HLB surfactant is PEG 40 hydrogenated castor oil. In some embodiments, the pharmaceutical composition further comprises a low HLB surfactant. In some embodiments, the low HLB surfactant is selected from the group consisting of propylene glycol monocaprylate, propylene glycol dicaprylocaprate, glyceryl monocaprylate, glyceryl monooleate, sorbitan monooleate, and a combination of two or more thereof. In some embodiments, the low HLB surfactant is propylene glycol monocaprylate. In some embodiments, the high HLB surfactant is present in the composition in an amount of about 50 wt. % to about 99.975 wt. %. In some embodiments, the high HLB surfactant is present in the composition in an amount of greater than 60 wt. % to about 99.975 wt. %. In some embodiments, the high HLB surfactant is present in the composition in an amount of greater than 60 wt. % to about 90 wt. %. In some embodiments, the high HLB surfactant is present in the composition in an amount of about 70 wt. % to about 85 wt. %. In some embodiments, the low HLB surfactant is present in the composition in an amount of about 10 wt. % to about 50 wt. %. In some embodiments, the low HLB surfactant is present in the composition in an amount of about 10 wt. % to less than 40 wt. %. In some embodiments, the low HLB surfactant is present in the composition in an amount of about 15 wt. % to about 30 wt. %. In some embodiments, wherein the low HLB surfactant is present in the composition in an amount of about 10 wt. % to about 25 wt. %. In some embodiments, the high HLB surfactant and the low HLB surfactant are present in the composition in a ratio of about 50:50 to about 90:10. In some embodiments, the high HLB surfactant and the low HLB surfactant are present in the composition in a ratio of greater than 60:40 to about 90:10. In some embodiments, the pharmaceutical composition further comprises one or more excipients selected from the group consisting of ethanol, isopropyl alcohol, benzyl alcohol, propylene glycol, N-methyl-2-pyrrolidone, glyceryol, dimethylsulfoxide, diethylene glycol monoethyl ether, poloxamer 124, and PEG 400. In some embodiments, the pharmaceutical composition further comprises one or more excipients selected from the group consisting of ethanol, diethylene glycol monoethyl ether, poloxamer 124, and PEG 400. In some embodiments, the one or more excipients are present in the composition in an amount of about 5 wt. % to about 10 wt. %; and the high HLB surfactant is present in the composition in an amount of greater than 54 wt. % to about 89.8 wt. %. In some embodiments, the one or more excipients are present in the composition in an amount of about 5 wt. % to about 10 wt. %; the high HLB surfactant and the low HLB surfactant are present in the composition in an total amount of about 89.8 wt. % to about 94.8 wt. %; and the high HLB surfactant and the low HLB surfactant are present in the composition in a ratio of greater than 60:40 to about 90:10.

In some embodiments, the one or more surfactants comprise a low HLB surfactant. In some embodiments, the low HLB surfactant is selected from the group consisting of propylene glycol monocaprylate, propylene glycol dicaprylocaprate, glyceryl monocaprylate, glyceryl monooleate, sorbitan monooleate, and a combination of two or more thereof. In some embodiments, the low HLB surfactant is propylene glycol monocaprylate. In some embodiments, the low HLB surfactant is present in the composition in an amount of about 10 wt. % to about 99.975 wt. %. In some embodiments, the pharmaceutical composition further comprises one or more excipients selected from the group consisting of ethanol, isopropyl alcohol, benzyl alcohol, propylene glycol, N-methyl-2-pyrrolidone, glyceryol, dimethylsulfoxide, diethylene glycol monoethyl ether, poloxamer 124, and PEG 400. In some embodiments, the one or more excipients are present in the composition in an amount of about 5 wt. % to about 10 wt. %; and the low HLB surfactant is present in the composition in an amount of about 9 wt. % to about 94.975 wt. %.

In some embodiments, the pharmaceutical composition comprises a compound of Formula I, or a stereoisomer or a tautomer thereof, or a pharmaceutically acceptable salt thereof, and PEG 40 hydrogenated castor oil. In some embodiments, the pharmaceutical composition comprises a compound of Formula I, or a stereoisomer or a tautomer thereof, or a pharmaceutically acceptable salt thereof; PEG 40 hydrogenated castor oil; and propylene glycol monocaprylate. In some embodiments, the pharmaceutical composition comprises a compound of Formula I, or a stereoisomer or a tautomer thereof, or a pharmaceutically acceptable salt thereof, and propylene glycol monocaprylate.

In some embodiments, the pharmaceutical composition consists essentially of a compound of Formula I, or a stereoisomer or a tautomer thereof, or a pharmaceutically acceptable salt thereof, and PEG 40 hydrogenated castor oil. In some embodiments, the pharmaceutical composition consists essentially of a compound of Formula I, or a stereoisomer or a tautomer thereof, or a pharmaceutically acceptable salt thereof; PEG 40 hydrogenated castor oil; and propylene glycol monocaprylate. In some embodiments, the pharmaceutical composition consists essentially of a compound of Formula I, or a stereoisomer or a tautomer thereof, or a pharmaceutically acceptable salt thereof, and propylene glycol monocaprylate.

In some embodiments, the pharmaceutical composition consists of a compound of Formula I, or a stereoisomer or a tautomer thereof, or a pharmaceutically acceptable salt thereof, and PEG 40 hydrogenated castor oil. In some embodiments, the pharmaceutical composition consists of a compound of Formula I, or a stereoisomer or a tautomer thereof, or a pharmaceutically acceptable salt thereof; PEG 40 hydrogenated castor oil; and propylene glycol monocaprylate. In some embodiments, the pharmaceutical composition consists of a compound of Formula I, or a stereoisomer or a tautomer thereof, or a pharmaceutically acceptable salt thereof, and propylene glycol monocaprylate.

In some embodiments, R¹ is C₁-C₆ alkyl, C₂-C₁₀ alkenyl, cyclopropyl, cyclobutyl, cyclopentyl, cyclohexyl, tetrahydrofuranyl, tetrahydropyranyl, azetidinyl, pyridazin-3(2H)-one, phenyl, naphthyl, pyridinyl, cinnolinyl, isoquinolinyl, quinolinyl, pyrazolo[1,5-a]pyridinyl, imidazo[1,5-a]pyridinyl, benzo[b]thiophenyl, a (cyclobutyl)alkyl group, a (cyclopentyl)alkyl group, a benzyl group, a (tetrahydrofuranyl)alkyl group, or a (tetrahydropyranyl)alkyl group; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen. In some embodiments, R¹ is a C₆-C₁₀ aryl group or a five- to ten-membered heteroaryl ring containing one to four ring heteroatoms independently selected from oxygen, sulfur, and nitrogen; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen. In some embodiments, R¹ is benzene optionally substituted with one to five R^{g} independently selected from the group consisting of hydroxy, halogen, CN, C₁-C₆ alkyl, C₁-C₆ alkoxy, and C₁-C₆ haloalkoxy, or two R^{g} together with the atoms to which they are attached form a ring, or two R^{g} together with the atoms to which they are attached form an aromatic or non-aromatic 3- to 6-membered ring, optionally containing one or two ring heteroatoms independently selected from oxygen, sulfur, and nitrogen. In some embodiments, R¹ is C₁-C₆ alkyl or a non-aromatic C₃-C₁₂ carbocyclic ring; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy. In some embodiments, R¹ is C₁-C₆ alkyl optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy. In some embodiments, R¹ is a non-aromatic C₃-C₁₂ carbocyclic ring optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy. In some embodiments, R³ is oxygen or —CH₂—. In some embodiments, Q is —CH—. In some embodiments, each of R⁴ and R⁵ is independently halogen or C₁-C₈ alkyl optionally substituted with one to five substituents independently selected from hydroxy, halogen, and C₁-C₆ alkoxy; and R⁶ is hydrogen; or R⁴ is halogen or C₁-C₅ alkyl optionally substituted with one to five substituents independently selected from hydroxy, halogen, and C₁-C₆ alkoxy; and R⁵ and R⁶ taken together, along with the carbon atoms to which they are attached, form a 4-, 5- or 6-membered carbocyclic ring.

In some embodiments, the compound is selected from the group consisting of:

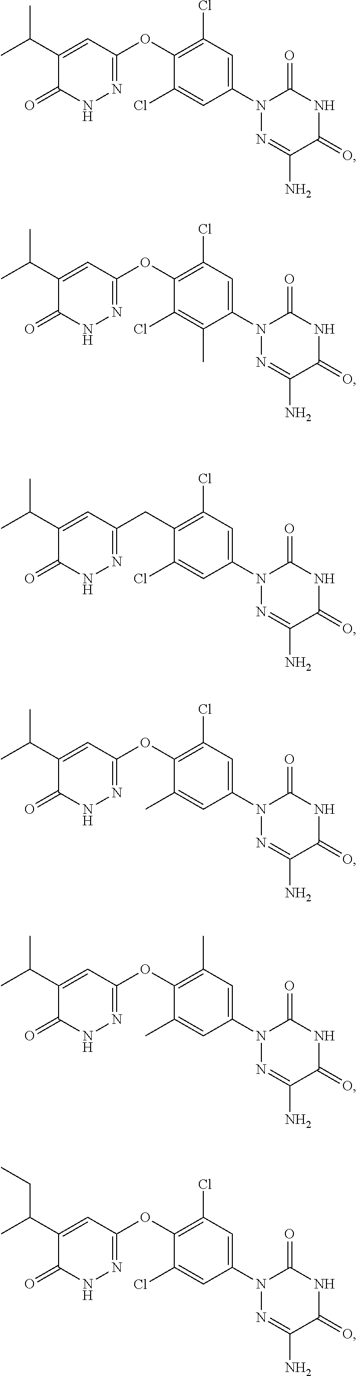 [SMILES: CC(C)c1cc(Oc2c(Cl)cc(-n3nc(N)c(=O)[nH]c3=O)cc2Cl)n[nH]c1=O ; Cc1c(-n2nc(N)c(=O)[nH]c2=O)cc(Cl)c(Oc2cc(C(C)C)c(=O)[nH]n2)c1Cl ; CC(C)c1cc(Cc2c(Cl)cc(-n3nc(N)c(=O)[nH]c3=O)cc2Cl)n[nH]c1=O ; Cc1cc(-n2nc(N)c(=O)[nH]c2=O)cc(Cl)c1Oc1cc(C(C)C)c(=O)[nH]n1 ; Cc1cc(-n2nc(N)c(=O)[nH]c2=O)cc(C)c1Oc1cc(C(C)C)c(=O)[nH]n1 ; CCC(C)c1cc(Oc2c(Cl)cc(-n3nc(N)c(=O)[nH]c3=O)cc2Cl)n[nH]c1=O]

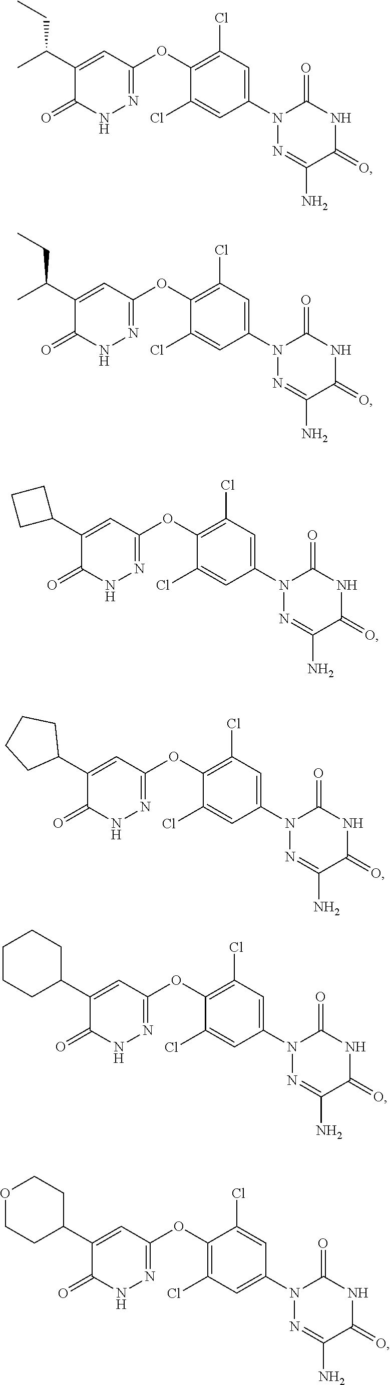 [SMILES: CC[C@@H](C)c1cc(Oc2c(Cl)cc(-n3nc(N)c(=O)[nH]c3=O)cc2Cl)n[nH]c1=O ; CC[C@H](C)c1cc(Oc2c(Cl)cc(-n3nc(N)c(=O)[nH]c3=O)cc2Cl)n[nH]c1=O ; Nc1nn(-c2cc(Cl)c(Oc3cc(C4CCC4)c(=O)[nH]n3)c(Cl)c2)c(=O)[nH]c1=O ; Nc1nn(-c2cc(Cl)c(Oc3cc(C4CCCC4)c(=O)[nH]n3)c(Cl)c2)c(=O)[nH]c1=O ; Nc1nn(-c2cc(Cl)c(Oc3cc(C4CCCCC4)c(=O)[nH]n3)c(Cl)c2)c(=O)[nH]c1=O ; Nc1nn(-c2cc(Cl)c(Oc3cc(C4CCOCC4)c(=O)[nH]n3)c(Cl)c2)c(=O)[nH]c1=O]

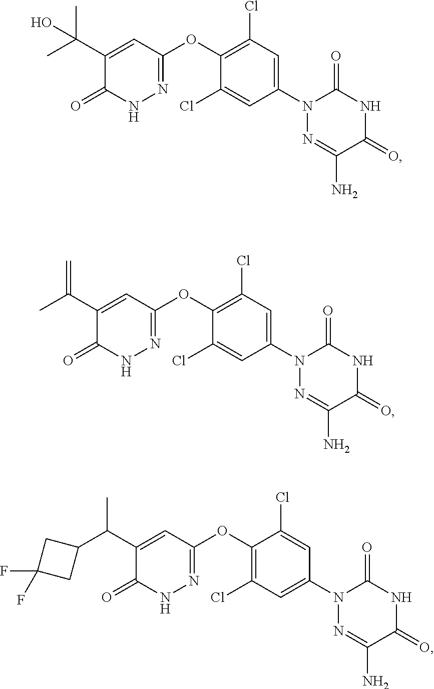 [SMILES: CC(C)(O)c1cc(Oc2c(Cl)cc(-n3nc(N)c(=O)[nH]c3=O)cc2Cl)n[nH]c1=O ; C=C(C)c1cc(Oc2c(Cl)cc(-n3nc(N)c(=O)[nH]c3=O)cc2Cl)n[nH]c1=O ; CC(c1cc(Oc2c(Cl)cc(-n3nc(N)c(=O)[nH]c3=O)cc2Cl)n[nH]c1=O)C1CC(F)(F)C1]

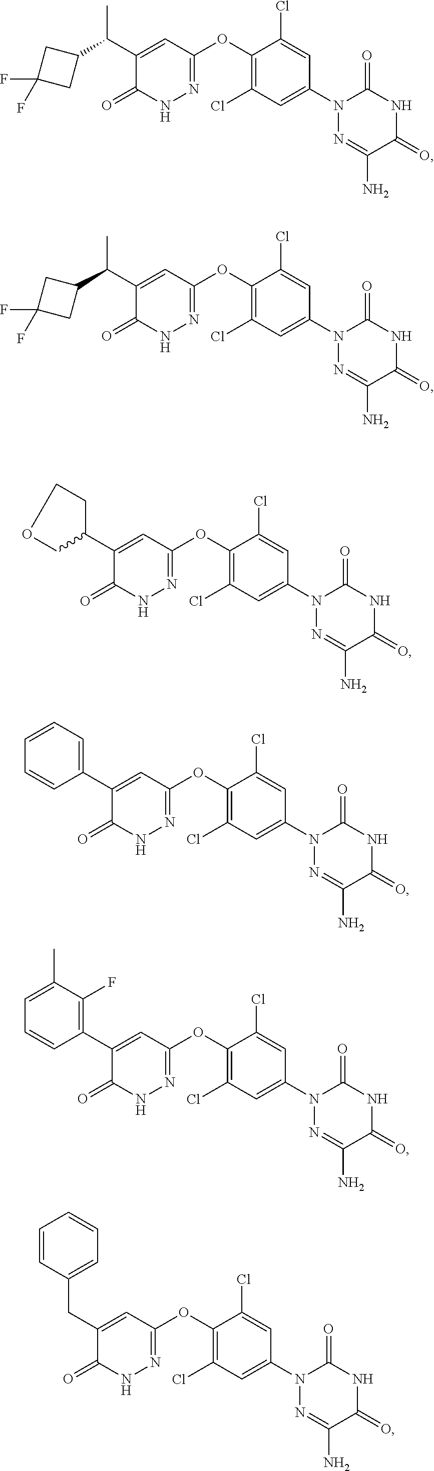 [SMILES: C[C@H](c1cc(Oc2c(Cl)cc(-n3nc(N)c(=O)[nH]c3=O)cc2Cl)n[nH]c1=O)C1CC(F)(F)C1 ; C[C@@H](c1cc(Oc2c(Cl)cc(-n3nc(N)c(=O)[nH]c3=O)cc2Cl)n[nH]c1=O)C1CC(F)(F)C1 ; Nc1nn(-c2cc(Cl)c(Oc3cc(C4CCOC4)c(=O)[nH]n3)c(Cl)c2)c(=O)[nH]c1=O ; Nc1nn(-c2cc(Cl)c(Oc3cc(-c4ccccc4)c(=O)[nH]n3)c(Cl)c2)c(=O)[nH]c1=O ; Cc1cccc(-c2cc(Oc3c(Cl)cc(-n4nc(N)c(=O)[nH]c4=O)cc3Cl)n[nH]c2=O)c1F ; Nc1nn(-c2cc(Cl)c(Oc3cc(Cc4ccccc4)c(=O)[nH]n3)c(Cl)c2)c(=O)[nH]c1=O]

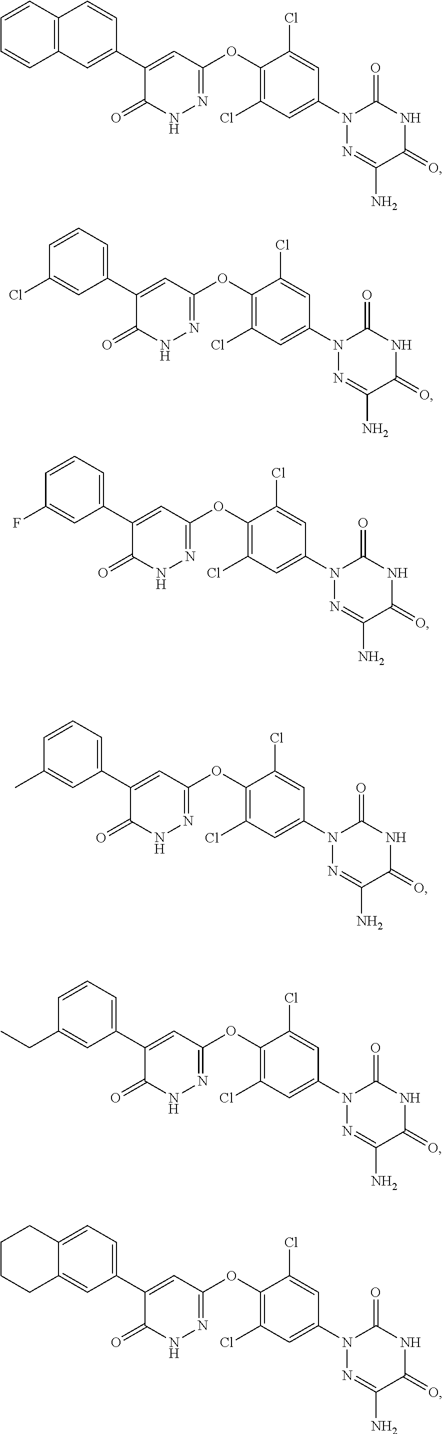 [SMILES: Nc1nn(-c2cc(Cl)c(Oc3cc(-c4ccc5ccccc5c4)c(=O)[nH]n3)c(Cl)c2)c(=O)[nH]c1=O ; Nc1nn(-c2cc(Cl)c(Oc3cc(-c4cccc(Cl)c4)c(=O)[nH]n3)c(Cl)c2)c(=O)[nH]c1=O ; Nc1nn(-c2cc(Cl)c(Oc3cc(-c4cccc(F)c4)c(=O)[nH]n3)c(Cl)c2)c(=O)[nH]c1=O ; Cc1cccc(-c2cc(Oc3c(Cl)cc(-n4nc(N)c(=O)[nH]c4=O)cc3Cl)n[nH]c2=O)c1 ; CCc1cccc(-c2cc(Oc3c(Cl)cc(-n4nc(N)c(=O)[nH]c4=O)cc3Cl)n[nH]c2=O)c1 ; Nc1nn(-c2cc(Cl)c(Oc3cc(-c4ccc5c(c4)CCCC5)c(=O)[nH]n3)c(Cl)c2)c(=O)[nH]c1=O]

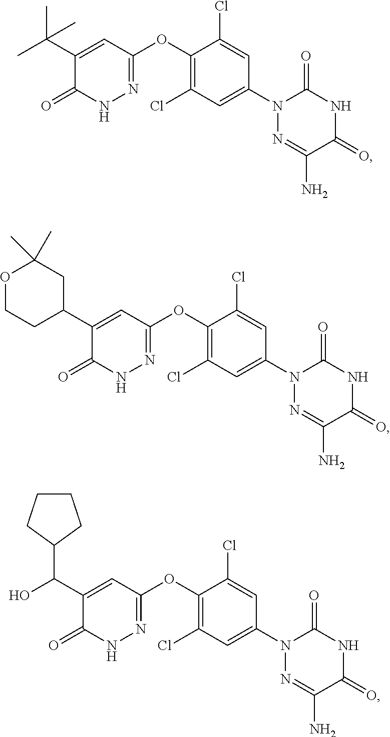 [SMILES: CC(C)(C)c1cc(Oc2c(Cl)cc(-n3nc(N)c(=O)[nH]c3=O)cc2Cl)n[nH]c1=O ; CC1(C)CC(c2cc(Oc3c(Cl)cc(-n4nc(N)c(=O)[nH]c4=O)cc3Cl)n[nH]c2=O)CCO1 ; Nc1nn(-c2cc(Cl)c(Oc3cc(C(O)C4CCCC4)c(=O)[nH]n3)c(Cl)c2)c(=O)[nH]c1=O]

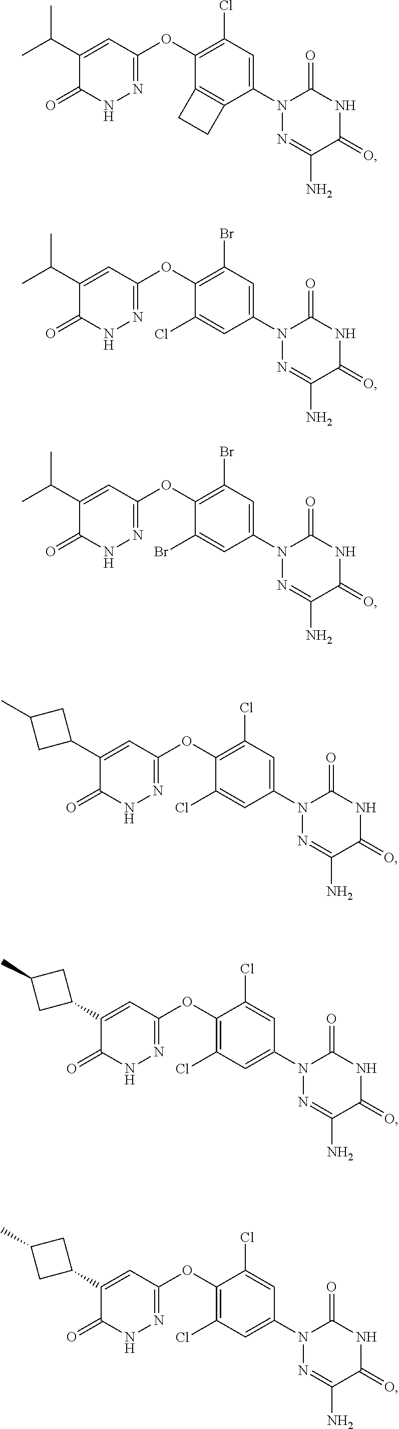 [SMILES: CC(C)c1cc(Oc2c(Cl)cc(-n3nc(N)c(=O)[nH]c3=O)c3c2CC3)n[nH]c1=O ; CC(C)c1cc(Oc2c(Cl)cc(-n3nc(N)c(=O)[nH]c3=O)cc2Br)n[nH]c1=O ; CC(C)c1cc(Oc2c(Br)cc(-n3nc(N)c(=O)[nH]c3=O)cc2Br)n[nH]c1=O ; CC1CC(c2cc(Oc3c(Cl)cc(-n4nc(N)c(=O)[nH]c4=O)cc3Cl)n[nH]c2=O)C1 ; C[C@H]1C[C@H](c2cc(Oc3c(Cl)cc(-n4nc(N)c(=O)[nH]c4=O)cc3Cl)n[nH]c2=O)C1 ; C[C@H]1C[C@@H](c2cc(Oc3c(Cl)cc(-n4nc(N)c(=O)[nH]c4=O)cc3Cl)n[nH]c2=O)C1]

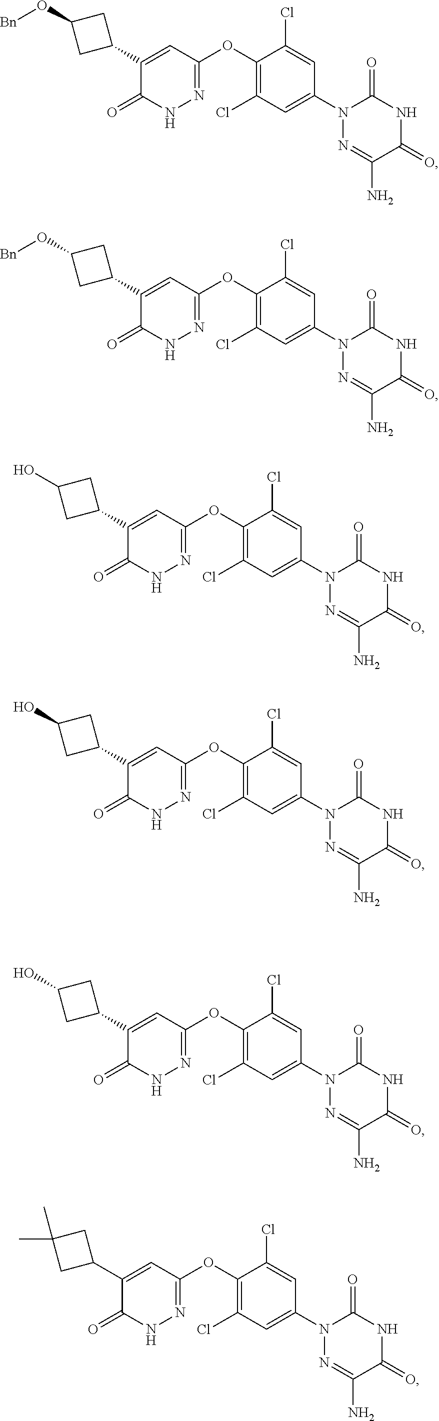

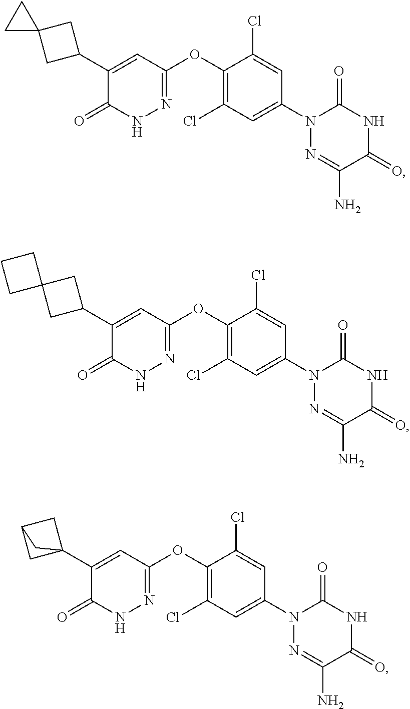 [SMILES: Nc1nn(-c2cc(Cl)c(Oc3cc(C4CC5(CC5)C4)c(=O)[nH]n3)c(Cl)c2)c(=O)[nH]c1=O ; Nc1nn(-c2cc(Cl)c(Oc3cc(C4CC5(CCC5)C4)c(=O)[nH]n3)c(Cl)c2)c(=O)[nH]c1=O ; Nc1nn(-c2cc(Cl)c(Oc3cc(C45CC(C4)C5)c(=O)[nH]n3)c(Cl)c2)c(=O)[nH]c1=O]

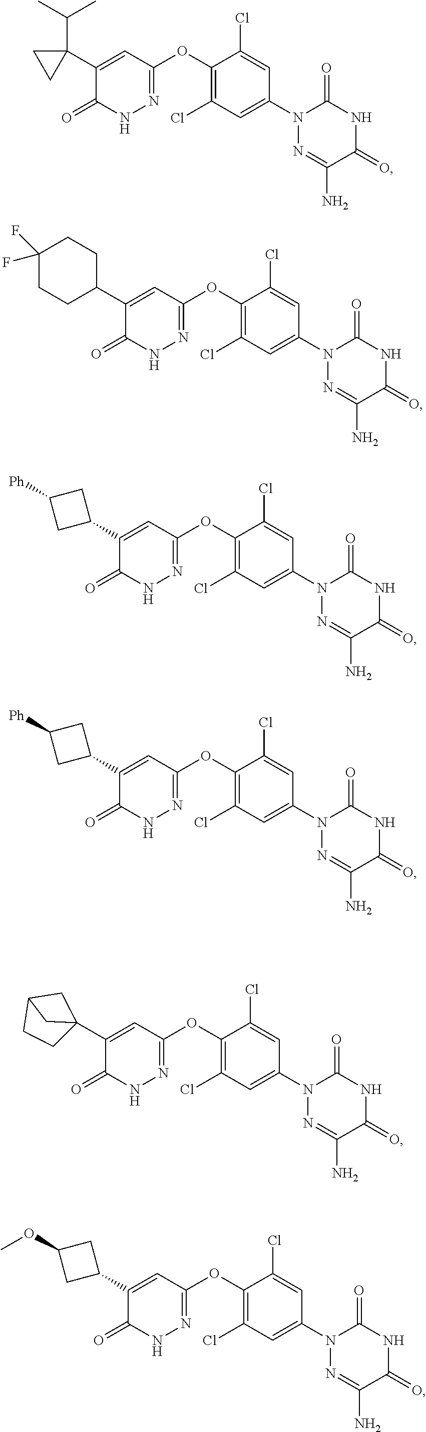 [SMILES: CC(C)C1(c2cc(Oc3c(Cl)cc(-n4nc(N)c(=O)[nH]c4=O)cc3Cl)n[nH]c2=O)CC1 ; Nc1nn(-c2cc(Cl)c(Oc3cc(C4CCC(F)(F)CC4)c(=O)[nH]n3)c(Cl)c2)c(=O)[nH]c1=O ; Nc1nn(-c2cc(Cl)c(Oc3cc([C@H]4C[C@@H](c5ccccc5)C4)c(=O)[nH]n3)c(Cl)c2)c(=O)[nH]c1=O ; Nc1nn(-c2cc(Cl)c(Oc3cc([C@H]4C[C@H](c5ccccc5)C4)c(=O)[nH]n3)c(Cl)c2)c(=O)[nH]c1=O ; Nc1nn(-c2cc(Cl)c(Oc3cc(C45CCC(C4)C5)c(=O)[nH]n3)c(Cl)c2)c(=O)[nH]c1=O ; CO[C@H]1C[C@H](c2cc(Oc3c(Cl)cc(-n4nc(N)c(=O)[nH]c4=O)cc3Cl)n[nH]c2=O)C1]

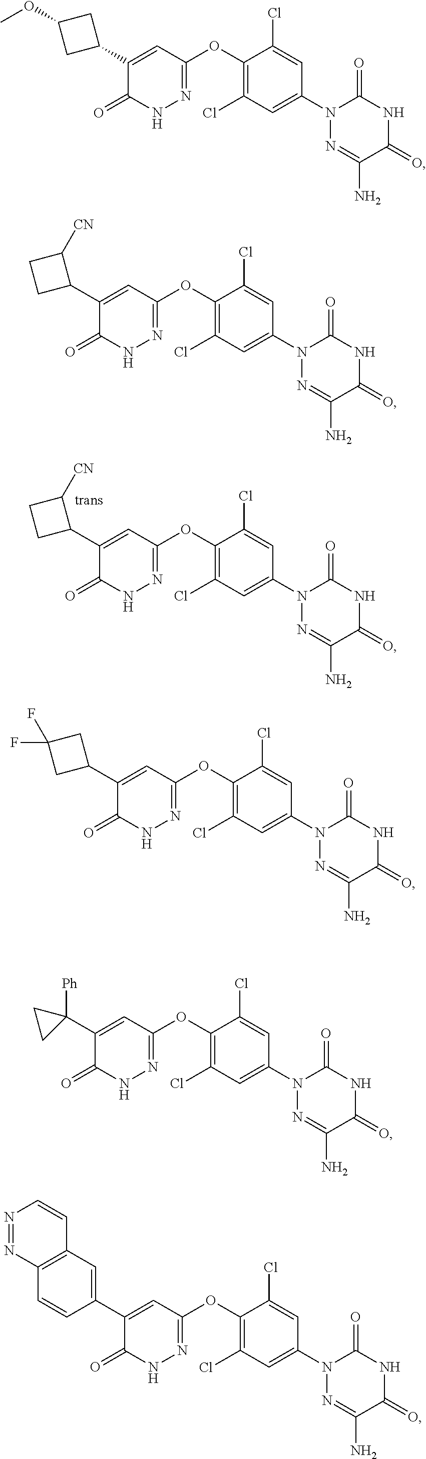 [SMILES: CO[C@H]1C[C@@H](c2cc(Oc3c(Cl)cc(-n4nc(N)c(=O)[nH]c4=O)cc3Cl)n[nH]c2=O)C1 ; N#CC1CCC1c1cc(Oc2c(Cl)cc(-n3nc(N)c(=O)[nH]c3=O)cc2Cl)n[nH]c1=O ; N#CC1CCC1c1cc(Oc2c(Cl)cc(-n3nc(N)c(=O)[nH]c3=O)cc2Cl)n[nH]c1=O ; Nc1nn(-c2cc(Cl)c(Oc3cc(C4CC(F)(F)C4)c(=O)[nH]n3)c(Cl)c2)c(=O)[nH]c1=O ; Nc1nn(-c2cc(Cl)c(Oc3cc(C4(c5ccccc5)CC4)c(=O)[nH]n3)c(Cl)c2)c(=O)[nH]c1=O ; Nc1nn(-c2cc(Cl)c(Oc3cc(-c4ccc5nnccc5c4)c(=O)[nH]n3)c(Cl)c2)c(=O)[nH]c1=O]

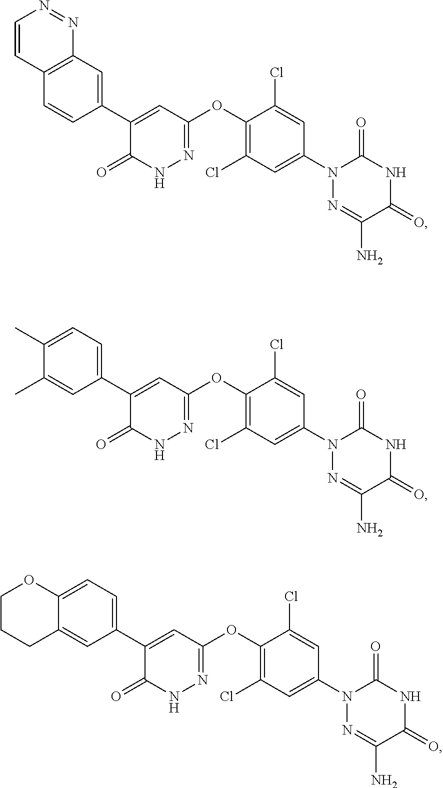 [SMILES: Nc1nn(-c2cc(Cl)c(Oc3cc(-c4ccc5ccnnc5c4)c(=O)[nH]n3)c(Cl)c2)c(=O)[nH]c1=O ; Cc1ccc(-c2cc(Oc3c(Cl)cc(-n4nc(N)c(=O)[nH]c4=O)cc3Cl)n[nH]c2=O)cc1C ; Nc1nn(-c2cc(Cl)c(Oc3cc(-c4ccc5c(c4)CCCO5)c(=O)[nH]n3)c(Cl)c2)c(=O)[nH]c1=O]

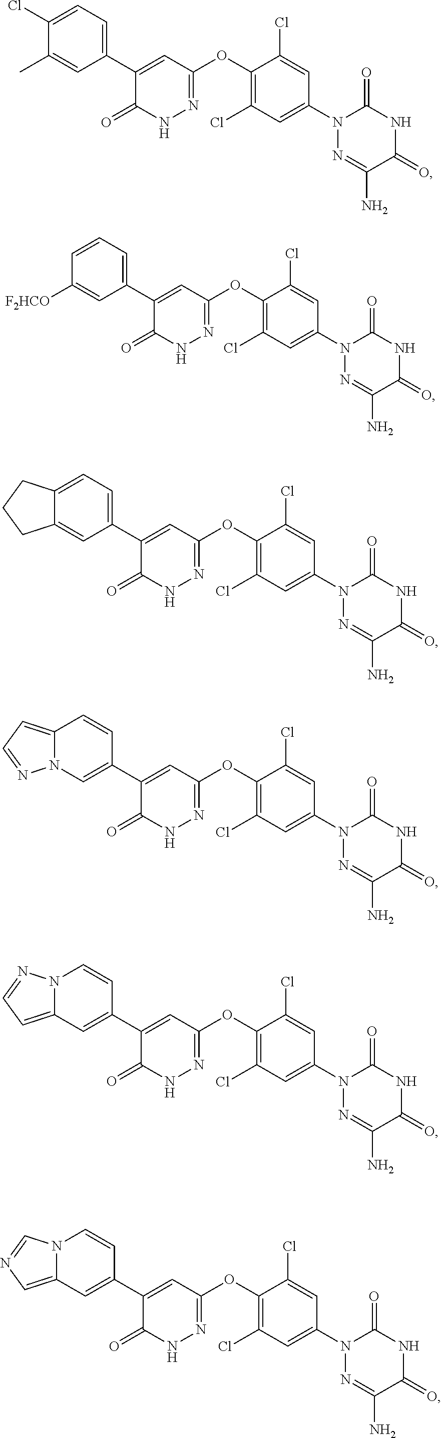 [SMILES: Cc1cc(-c2cc(Oc3c(Cl)cc(-n4nc(N)c(=O)[nH]c4=O)cc3Cl)n[nH]c2=O)ccc1Cl ; Nc1nn(-c2cc(Cl)c(Oc3cc(-c4cccc(OC(F)F)c4)c(=O)[nH]n3)c(Cl)c2)c(=O)[nH]c1=O ; Nc1nn(-c2cc(Cl)c(Oc3cc(-c4ccc5c(c4)CCC5)c(=O)[nH]n3)c(Cl)c2)c(=O)[nH]c1=O ; Nc1nn(-c2cc(Cl)c(Oc3cc(-c4ccc5ccnn5c4)c(=O)[nH]n3)c(Cl)c2)c(=O)[nH]c1=O ; Nc1nn(-c2cc(Cl)c(Oc3cc(-c4ccn5nccc5c4)c(=O)[nH]n3)c(Cl)c2)c(=O)[nH]c1=O ; Nc1nn(-c2cc(Cl)c(Oc3cc(-c4ccn5cncc5c4)c(=O)[nH]n3)c(Cl)c2)c(=O)[nH]c1=O]

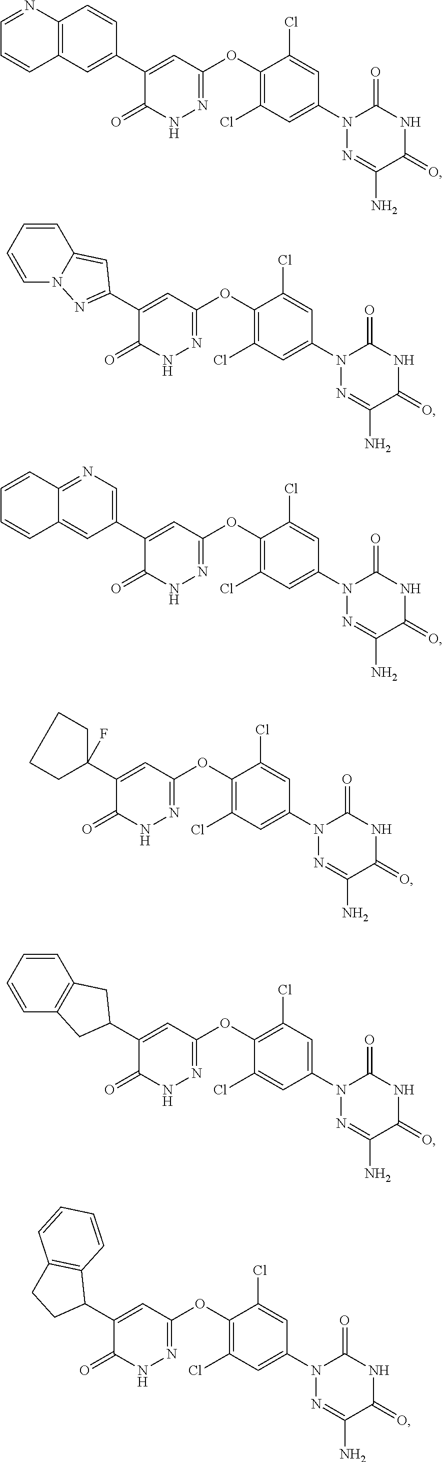 [SMILES: Nc1nn(-c2cc(Cl)c(Oc3cc(-c4ccc5ncccc5c4)c(=O)[nH]n3)c(Cl)c2)c(=O)[nH]c1=O ; Nc1nn(-c2cc(Cl)c(Oc3cc(-c4cc5ccccn5n4)c(=O)[nH]n3)c(Cl)c2)c(=O)[nH]c1=O ; Nc1nn(-c2cc(Cl)c(Oc3cc(-c4cnc5ccccc5c4)c(=O)[nH]n3)c(Cl)c2)c(=O)[nH]c1=O ; Nc1nn(-c2cc(Cl)c(Oc3cc(C4(F)CCCC4)c(=O)[nH]n3)c(Cl)c2)c(=O)[nH]c1=O ; Nc1nn(-c2cc(Cl)c(Oc3cc(C4Cc5ccccc5C4)c(=O)[nH]n3)c(Cl)c2)c(=O)[nH]c1=O ; Nc1nn(-c2cc(Cl)c(Oc3cc(C4CCc5ccccc54)c(=O)[nH]n3)c(Cl)c2)c(=O)[nH]c1=O]

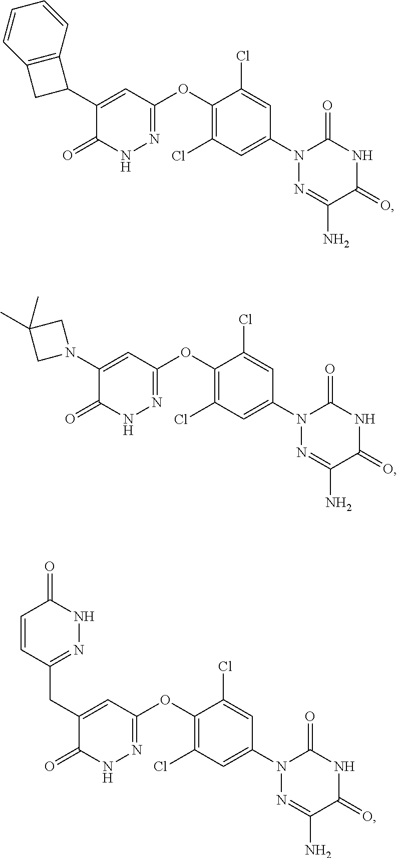 [SMILES: Nc1nn(-c2cc(Cl)c(Oc3cc(C4Cc5ccccc54)c(=O)[nH]n3)c(Cl)c2)c(=O)[nH]c1=O ; CC1(C)CN(c2cc(Oc3c(Cl)cc(-n4nc(N)c(=O)[nH]c4=O)cc3Cl)n[nH]c2=O)C1 ; Nc1nn(-c2cc(Cl)c(Oc3cc(Cc4ccc(=O)[nH]n4)c(=O)[nH]n3)c(Cl)c2)c(=O)[nH]c1=O]

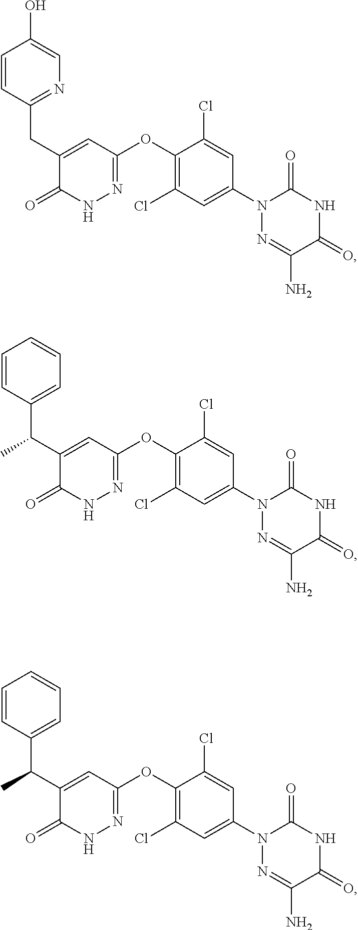 [SMILES: Nc1nn(-c2cc(Cl)c(Oc3cc(Cc4ccc(O)cn4)c(=O)[nH]n3)c(Cl)c2)c(=O)[nH]c1=O ; C[C@H](c1ccccc1)c1cc(Oc2c(Cl)cc(-n3nc(N)c(=O)[nH]c3=O)cc2Cl)n[nH]c1=O ; C[C@@H](c1ccccc1)c1cc(Oc2c(Cl)cc(-n3nc(N)c(=O)[nH]c3=O)cc2Cl)n[nH]c1=O]

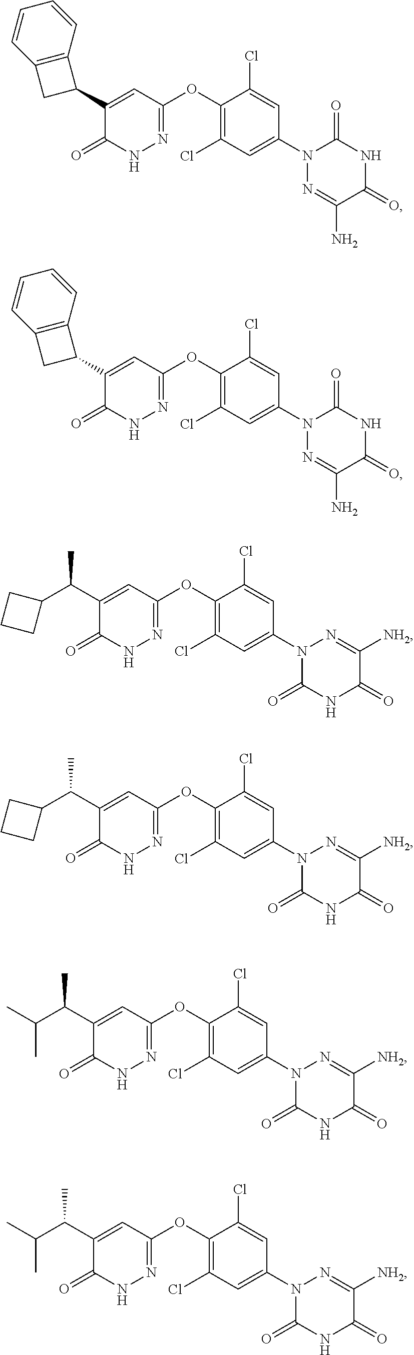 [SMILES: Nc1nn(-c2cc(Cl)c(Oc3cc([C@H]4Cc5ccccc54)c(=O)[nH]n3)c(Cl)c2)c(=O)[nH]c1=O ; Nc1nn(-c2cc(Cl)c(Oc3cc([C@@H]4Cc5ccccc54)c(=O)[nH]n3)c(Cl)c2)c(=O)[nH]c1=O ; C[C@@H](c1cc(Oc2c(Cl)cc(-n3nc(N)c(=O)[nH]c3=O)cc2Cl)n[nH]c1=O)C1CCC1 ; C[C@H](c1cc(Oc2c(Cl)cc(-n3nc(N)c(=O)[nH]c3=O)cc2Cl)n[nH]c1=O)C1CCC1 ; CC(C)[C@@H](C)c1cc(Oc2c(Cl)cc(-n3nc(N)c(=O)[nH]c3=O)cc2Cl)n[nH]c1=O ; CC(C)[C@H](C)c1cc(Oc2c(Cl)cc(-n3nc(N)c(=O)[nH]c3=O)cc2Cl)n[nH]c1=O]

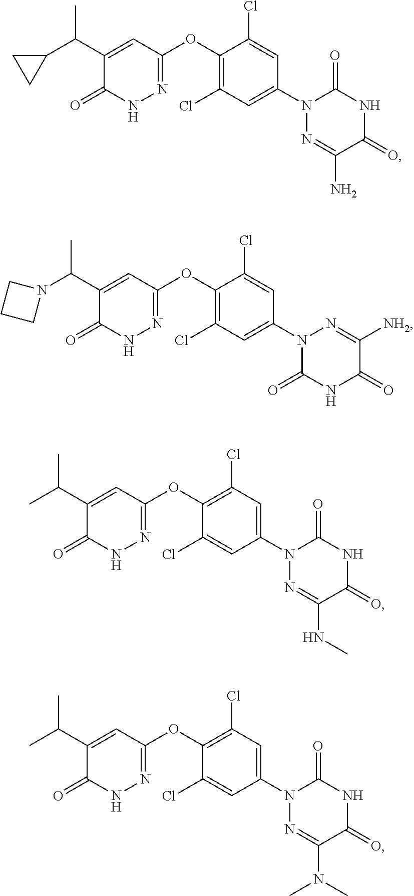 [SMILES: CC(c1cc(Oc2c(Cl)cc(-n3nc(N)c(=O)[nH]c3=O)cc2Cl)n[nH]c1=O)C1CC1 ; CC(c1cc(Oc2c(Cl)cc(-n3nc(N)c(=O)[nH]c3=O)cc2Cl)n[nH]c1=O)N1CCC1 ; CNc1nn(-c2cc(Cl)c(Oc3cc(C(C)C)c(=O)[nH]n3)c(Cl)c2)c(=O)[nH]c1=O ; CC(C)c1cc(Oc2c(Cl)cc(-n3nc(N(C)C)c(=O)[nH]c3=O)cc2Cl)n[nH]c1=O]

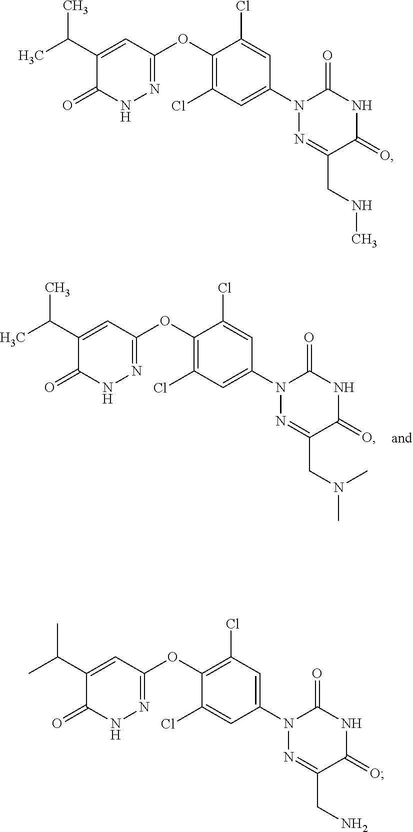

or a stereoisomer or a tautomer thereof, or a pharmaceutically acceptable salt thereof.

In some embodiments, R¹ is unsubstituted C₁-C₆ alkyl or unsubstituted C₃-C₆ carbocyclic ring; R² is hydrogen; R³ is —CH₂— or oxygen; Q is —CH—; R⁷ is NH₂ and (i) each of R⁴ and R⁵ is independently selected from halogen and C₁-C₆ alkyl, and R⁶ is hydrogen or C₁-C₆ alkyl, or (ii) R⁴ is halogen, and R⁵ and R⁶ taken together, along with the carbon atoms to which they are attached, form a 4-, 5- or 6-membered non-aromatic carbocyclic ring. In some embodiments, the compound is selected from the group consisting of:

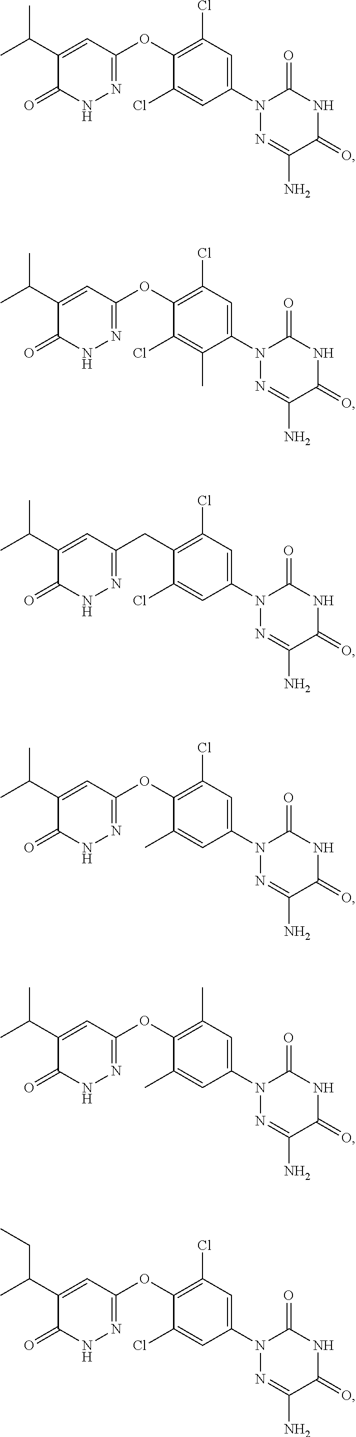 [SMILES: CC(C)c1cc(Oc2c(Cl)cc(-n3nc(N)c(=O)[nH]c3=O)cc2Cl)n[nH]c1=O ; Cc1c(-n2nc(N)c(=O)[nH]c2=O)cc(Cl)c(Oc2cc(C(C)C)c(=O)[nH]n2)c1Cl ; CC(C)c1cc(Cc2c(Cl)cc(-n3nc(N)c(=O)[nH]c3=O)cc2Cl)n[nH]c1=O ; Cc1cc(-n2nc(N)c(=O)[nH]c2=O)cc(Cl)c1Oc1cc(C(C)C)c(=O)[nH]n1 ; Cc1cc(-n2nc(N)c(=O)[nH]c2=O)cc(C)c1Oc1cc(C(C)C)c(=O)[nH]n1 ; CCC(C)c1cc(Oc2c(Cl)cc(-n3nc(N)c(=O)[nH]c3=O)cc2Cl)n[nH]c1=O]

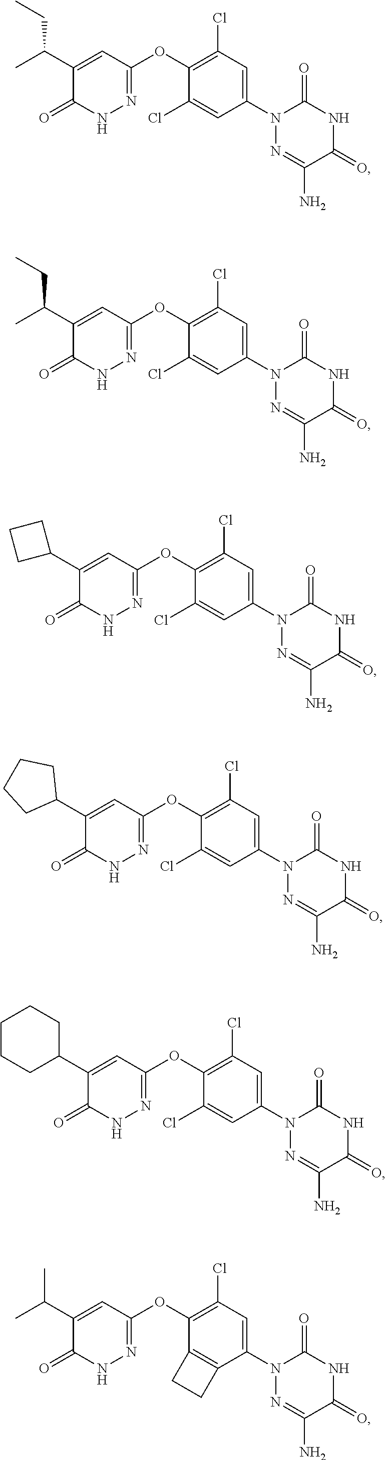 [SMILES: CC[C@@H](C)c1cc(Oc2c(Cl)cc(-n3nc(N)c(=O)[nH]c3=O)cc2Cl)n[nH]c1=O ; CC[C@H](C)c1cc(Oc2c(Cl)cc(-n3nc(N)c(=O)[nH]c3=O)cc2Cl)n[nH]c1=O ; Nc1nn(-c2cc(Cl)c(Oc3cc(C4CCC4)c(=O)[nH]n3)c(Cl)c2)c(=O)[nH]c1=O ; Nc1nn(-c2cc(Cl)c(Oc3cc(C4CCCC4)c(=O)[nH]n3)c(Cl)c2)c(=O)[nH]c1=O ; Nc1nn(-c2cc(Cl)c(Oc3cc(C4CCCCC4)c(=O)[nH]n3)c(Cl)c2)c(=O)[nH]c1=O ; CC(C)c1cc(Oc2c(Cl)cc(-n3nc(N)c(=O)[nH]c3=O)c3c2CC3)n[nH]c1=O]

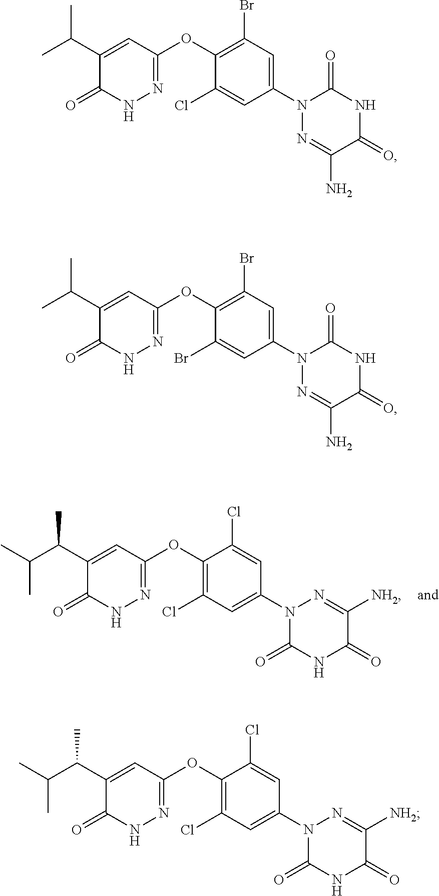 [SMILES: CC(C)c1cc(Oc2c(Cl)cc(-n3nc(N)c(=O)[nH]c3=O)cc2Br)n[nH]c1=O ; CC(C)c1cc(Oc2c(Br)cc(-n3nc(N)c(=O)[nH]c3=O)cc2Br)n[nH]c1=O ; CC(C)[C@@H](C)c1cc(Oc2c(Cl)cc(-n3nc(N)c(=O)[nH]c3=O)cc2Cl)n[nH]c1=O ; CC(C)[C@H](C)c1cc(Oc2c(Cl)cc(-n3nc(N)c(=O)[nH]c3=O)cc2Cl)n[nH]c1=O]

or a stereoisomer or a tautomer thereof, or a pharmaceutically acceptable salt thereof. In some embodiments, the pharmaceutical composition consists of:

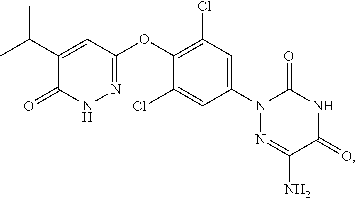 [SMILES: CC(C)c1cc(Oc2c(Cl)cc(-n3nc(N)c(=O)[nH]c3=O)cc2Cl)n[nH]c1=O]

or a pharmaceutically acceptable salt thereof, in an amount of from about 0.1 wt. % to about 0.3 wt. %; and propylene glycol monocaprylate and PEG 40 hydrogenated castor oil in a ratio of from about 30:70 to about 20:80 (propylene glycol monocaprylate:PEG 40 hydrogenated castor oil). In some embodiments,

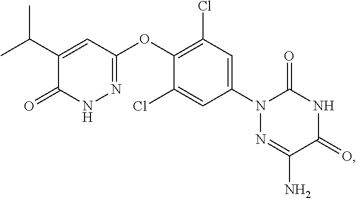 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of about 0.2 wt. %. In some embodiments,

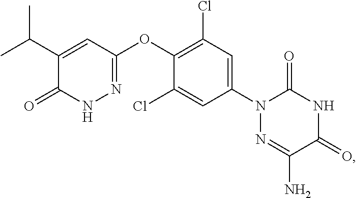 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of about 0.2 wt. %, and propylene glycol monocaprylate and PEG 40 hydrogenated castor oil are present in a ratio of 25:75 (propylene glycol monocaprylate:PEG 40 hydrogenated castor oil). In some embodiments,

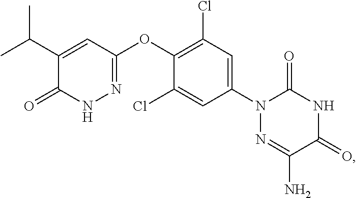 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of from about 100 μg to about 2000 μg. In some embodiments,

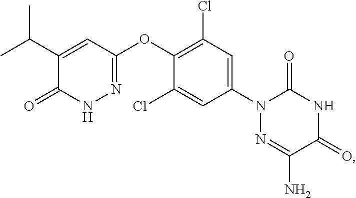 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of about 300, 500, 700, 900, or 1200 μg.

In some embodiments, the composition is in the form of a capsule. In some embodiments, the capsule is a soft gel capsule.

Provided herein, in another aspect, is a method of treating a disorder or disease in a subject in need thereof, the method comprising administering to the subject a therapeutically effective amount of a pharmaceutical composition disclosed herein, wherein the disorder or disease is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer. In some embodiments, the pharmaceutical composition described herein is administered in combination with a KHK inhibitor, an FXR agonist, a SSAO inhibitor, a FASN inhibitor, or a SCD1 modulator. In some embodiments, the KHK inhibitor is PF-06835919; the FXR agonist is TERN-101 (LY2562175), Tropifexor, obeticholic acid (OCA), or ASC42; the SSAO inhibitor is TERN-201; the FASN inhibitor is ASC40; and the SCD1 modulator is aramchol.

Provided herein, in another aspect, is a use of a pharmaceutical composition disclosed herein for the manufacture of a medicament for the treatment of a disorder or disease is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a composition disclosed herein for use in treating a disorder or disease is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a method of treating a thyroid hormone receptor related disorder in a patient, the method comprising the steps of identifying a patient in need of treatment for the thyroid hormone receptor related disorder, and administering to the patient, or contacting the patient with, a therapeutically effective amount of a pharmaceutical composition disclosed herein. In some embodiments, the thyroid hormone receptor related disorder is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

Provided herein, in another aspect, is a soft gel capsule comprising a fill formulation and a capsule shell, wherein: the fill formulation comprises a compound of Formula I, or a stereoisomer or a tautomer thereof, or a pharmaceutically acceptable salt thereof, in an amount of from about 0.1 wt. % to about 0.3 wt. % of the fill formulation; and propylene glycol monocaprylate and PEG 40 hydrogenated castor oil in a ratio of from about 30:70 to about 20:80 (propylene glycol monocaprylate:PEG 40 hydrogenated castor oil); and the capsule shell is prepared from an initial composition comprising gelatin, glycerol, dye, titanium dioxide, and purified water; and the initial composition undergoes drying to substantially remove the purified water to prepare the capsule shell.

Provided herein, in another aspect, is a soft gel capsule comprising a fill formulation and a capsule shell, wherein: the fill formulation comprises a compound of Formula I, or a stereoisomer or a tautomer thereof, or a pharmaceutically acceptable salt thereof, in an amount of from about 0.1 wt. % to about 0.3 wt. % of the fill formulation; and propylene glycol monocaprylate and PEG 40 hydrogenated castor oil in a ratio of from about 30:70 to about 20:80 (propylene glycol monocaprylate:PEG 40 hydrogenated castor oil); and the capsule shell is prepared from an initial composition comprising gelatin in an amount of from about 41 wt. % to about 48 wt. % of the initial composition; glycerol in an amount of from about 20 wt. % to about 25 wt. % of the initial composition; dye in an amount of about 0.02 wt. % of the initial composition; titanium dioxide in an amount of about 1 wt. % of the initial composition; and purified water in an amount of from about 29 wt. % to about 33 wt. % of the initial composition; and the initial composition undergoes drying to substantially remove the purified water to prepare the capsule shell. In some embodiments, propylene glycol monocaprylate and PEG 40 hydrogenated castor oil are present in the fill formulation in a ratio of about 30:70, about 25:75, or about 20:80 (propylene glycol monocaprylate:PEG 40 hydrogenated castor oil).

Provided herein, in another aspect, is a soft gel capsule comprising a fill formulation and a capsule shell, wherein: the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

Provided herein, in another aspect, is a soft gel capsule comprising a fill formulation and a capsule shell, wherein: the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and the capsule shell is prepared from an initial composition comprising gelatin in an amount of from about 35 wt. % to about 45 wt. % of the initial composition; the mixture of sorbitol and sorbitan in an amount of from about 8 wt. % to about 15 wt. % of the initial composition; glycerin in an amount of from about 8 wt. % to about 15 wt. % of the initial composition; titanium dioxide in an amount of from about 1 wt. % to about 2 wt. % of the initial composition; and purified water in an amount of from about 30 wt. % to about 40 wt. % of the initial composition; and the initial composition undergoes drying to substantially remove the purified water to prepare the capsule shell.

In some embodiments, the compound of Formula I, or a stereoisomer or a tautomer thereof, or a pharmaceutically acceptable salt thereof, in an amount of from about 0.05 wt. % to about 0.4 wt. % of the fill formulation. In some embodiments, the PEG 40 hydrogenated castor oil and the propylene glycol monocaprylate are present in the fill formulation in a ratio of from about 70:30 to about 80:20 (PEG 40 hydrogenated castor oil:propylene glycol monocaprylate). In some embodiments, the PEG 40 hydrogenated castor oil and the propylene glycol monocaprylate are present in the fill formulation in a ratio of about 75:25 (PEG 40 hydrogenated castor oil:propylene glycol monocaprylate). In some embodiments, the gelatin is 160-175 Bloom gelatin. In some embodiments, the gelatin is 175 Bloom gelatin. In some embodiments, the gelatin is limed bone gelatin.

Provided herein, in another aspect, is a method to prepare a soft gel capsule, wherein the method comprises: combining (i) a compound of Formula I, or a stereoisomer or a tautomer thereof, or a pharmaceutically acceptable salt thereof; (ii) propylene glycol monocaprylate; and (iii) PEG 40 hydrogenated castor oil under oxygen-free conditions to form a fill formulation; combining gelatin, a mixture of sorbitol and sorbitan, glycerin, and titanium dioxide in purified water to provide a gelatin mass; and encapsulating the fill formulation within the gelatin mass followed by drying to form individual soft gel capsules. In some embodiments, the compound of Formula I, or the stereoisomer or the tautomer thereof, or the pharmaceutically acceptable salt thereof, is present in the fill formulation in an amount of from about 0.025 wt. % to about 1.0 wt. %. In some embodiments, the compound of Formula I, or the stereoisomer or the tautomer thereof, or the pharmaceutically acceptable salt thereof, is present in the fill formulation in an amount of from about 0.05 wt. % to about 0.4 wt. %. In some embodiments, the PEG 40 hydrogenated castor oil and the propylene glycol monocaprylate are present in the fill formulation in a ratio of from about 50:50 to about 90:10 (PEG 40 hydrogenated castor oil:propylene glycol monocaprylate). In some embodiments, the PEG 40 hydrogenated castor oil and the propylene glycol monocaprylate are present in the fill formulation in a ratio of from about 70:30 to about 80:20 (PEG 40 hydrogenated castor oil:propylene glycol monocaprylate). In some embodiments, the PEG 40 hydrogenated castor oil and the propylene glycol monocaprylate are present in the fill formulation in a ratio of about 75:25 (PEG 40 hydrogenated castor oil:propylene glycol monocaprylate). In some embodiments, the gelatin is 160-175 Bloom gelatin. In some embodiments, the gelatin is 175 Bloom gelatin. In some embodiments, the gelatin is limed bone gelatin.

Provided herein, in another aspect, is a soft gel capsule prepared by the method described herein.

### BRIEF DESCRIPTION OF THE DRAWINGS

FIG. 1 depicts mean (+SD) plasma concentration-time profiles of compound A following single oral doses of solution vs solid formulations in fasted condition in healthy subjects (log-linear scale).

FIG. 2 depicts mean (+SD) plasma concentration-time profiles of compound A following single oral doses of solid formulation in fasted conditions in healthy subjects (log-linear scale).

FIG. 3 depicts in vitro dissolution data for soft gel capsules containing compound A after storage at 25° C. and 60% relative humidity.

FIG. 4 depicts in vitro dissolution data for soft gel capsules containing compound A after storage at 40° C. and 75% relative humidity.

FIG. 5A and FIG. 5B depict in vitro dissolution data for soft gel capsules containing compound A with a capsule shell containing 175 Bloom limed bone gelatin (FIG. 5A) or 166 Bloom gelatin STABICAPS™ (FIG. 5B) after storage at 40° C. and 75% relative humidity.

### DETAILED DESCRIPTION

### Definitions

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

In the definition of chemical substituents, each of Rₓ and R_{y} is independently hydrogen, alkyl, carbocyclic ring, heterocyclic ring, aryl, or heteroaryl, all of which, except hydrogen, are optionally substituted.

Unless otherwise indicated, the abbreviations “TR” and “THR” refer to thyroid hormone receptors.

As used herein, “pharmaceutically acceptable salt” refers to a salt of a compound that does not cause significant irritation to a patient to which it is administered and does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methane-sulfonates, ethanesulfonates, p-toluenesulfonates and salicylates.

As used herein, “pharmaceutically acceptable ester” refers to an ester of a compound that does not cause significant irritation to a patient to which it is administered. The ester is metabolized in the body to result in the parent compound, e.g., the claimed compound. Accordingly, the ester does not abrogate the biological activity and properties of the compound. Pharmaceutical esters can be obtained by reaction of a compound disclosed herein with an alcohol. Methyl, ethyl, and isopropyl esters are some of the common esters to be prepared. Other esters suitable are well-known to those skilled in the art (see, for example Wuts, P. G. M., Greene's Protective Groups in Organic Synthesis, 5^{th} Ed., John Wiley & Sons, New York, N.Y., 2014, which is incorporated herein by reference in its entirety).

Where the compounds disclosed herein have at least one chiral center, they may exist as a racemate or as individual enantiomers. It should be noted that all such isomers and mixtures thereof are included in the scope of the present disclosure. Thus, the illustration of a chiral center without a designation of R or S signifies that the scope of the disclosure includes the R isomer, the S isomer, the racemic mixture of the isomers, or mixtures where one isomer is present in greater abundance than the other.

Where the processes for the preparation of the compounds disclosed herein give rise to mixtures of stereoisomers, such isomers may be separated by conventional techniques such as preparative chiral chromatography. The compounds may be prepared in racemic form or individual enantiomers may be prepared by stereoselective synthesis or by resolution. The compounds may be resolved into their component enantiomers by standard techniques, such as the formation of diastereomeric pairs by salt formation with an optically active acid, such as (−)-di-p-toluoyl-d-tartaric acid and/or (+)-di-p-toluoyl-l-tartaric acid, followed by fractional crystallization and regeneration of the free base. The compounds may also be resolved by formation of diastereomeric esters or amides followed by chromatographic separation and removal of the chiral auxiliary.

Unless otherwise indicated, when a substituent is deemed to be “optionally substituted” it is meant that the substituent is a group that may be substituted with one or more (e.g., 1 or 2, or 1 to 3, or 1 to 4 or 1 to 5, or 1 to 6) group(s) individually and independently selected, without limitation, from alkyl, alkenyl, alkynyl, cycloalkyl, aryl, heteroaryl, heterocycloalkyl, hydroxy, alkoxy, aryloxy, mercapto, alkylthio, arylthio, cyano, halo, carbonyl, thiocarbonyl, O-carbamoyl, N-carbamoyl, O-thiocarbamoyl, N-thiocarbamoyl, C-amido, N-amido, S-sulfonamido, N-sulfonamido, C-carboxy, O-carboxy, is O-cyanato, thiocyanato, isothiocyanato, nitro, silyl, trihalomethanesulfonyl, and amino (e.g., —NRₓR_{y}), including mono- and di-substituted amino groups, and the protected derivatives thereof. The protecting groups that may form the protective derivatives of the above substituents are known to those of skill in the art and may be found in references such as Wuts, above.

As used herein, a “carbocyclic ring” is an aromatic or non-aromatic ring structure in which all the atoms in the ring are carbon atoms. As such, the ring structure may be fully saturated, fully unsaturated, or partially saturated. If any of the atoms in the ring is anything other than a carbon atom, then the ring is a “heterocyclic ring.” Examples of atoms that are within a ring include sulfur, oxygen, and nitrogen. A carbocyclic ring or a heterocyclic ring may be polycyclic, e.g., a fused ring system, a spirocyclic ring system, or a bridged ring system. These polycyclic rings include, for example, adamantyl, norbornyl (i.e., bicyclo[2.2.1]heptanyl), norbornenyl, decalinyl, 7,7-dimethyl-bicyclo[2.2.1]heptanyl, and the like. Additional non-limiting examples include bicyclic rings such as but not limited to:

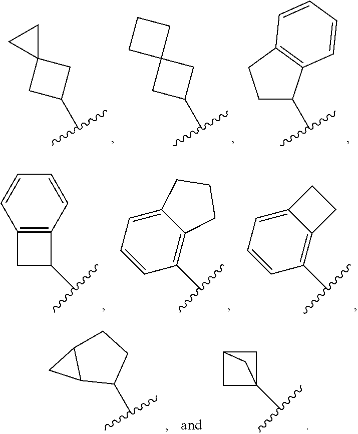

As used herein, “aryl” refers to a carbocyclic (all carbon) ring that has a fully delocalized pi-electron system. The “aryl” group can be made up of two or more fused rings (rings that share two adjacent carbon atoms). When the aryl is a fused ring system, then the ring that is connected to the rest of the molecule has a fully delocalized pi-electron system. The other ring(s) in the fused ring system may or may not have a fully delocalized pi-electron system. Further, the other ring(s) may or may not contain one or more heteroatoms (e.g., O, N, or S). Examples of aryl groups include, without limitation, the radicals of benzene, naphthalene and azulene. Additional non-limiting examples include:

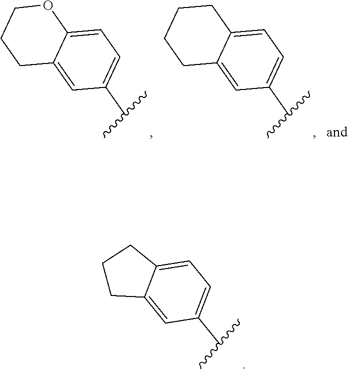 [SMILES: *c1ccc2c(c1)CCCO2 ; *c1ccc2c(c1)CCCC2 ; *c1ccc2c(c1)CCC2]

As used herein, “heteroaryl” refers to a ring that has a fully delocalized pi-electron system and contains one or more heteroatoms selected from the group consisting of nitrogen, oxygen, and sulfur in the ring. The “heteroaryl” group can be made up of two or more fused rings (rings that share two adjacent carbon atoms). When the heteroaryl is a fused ring system, then the ring that is connected to the rest of the molecule has a fully delocalized pi-electron system. The other ring(s) in the fused ring system may or may not have a fully delocalized pi-electron system. Examples of heteroaryl rings include, without limitation, furan, thiophene, phthalazinone, pyrrole, oxazole, thiazole, imidazole, pyrazole, isoxazole, isothiazole, triazole, thiadiazole, pyridine, pyridazine, pyrimidine, pyrazine and triazine.

Wherever “hetero” is used it is intended to mean a group as specified, such as an alkyl or an aryl group, where at least one carbon atom has been replaced with a heteroatom selected from nitrogen, oxygen and sulfur.

As used herein, “alkyl” refers to a straight or branched chain fully saturated (no double or triple bonds) hydrocarbon group. An alkyl group of the presently disclosed compounds may comprise from 1 to 20 carbon atoms. An alkyl group herein may also be of medium size having 1 to 10 carbon atoms. An alkyl group herein may also be a lower alkyl having 1 to 5 carbon atoms or 1 to 6 carbon atoms. Examples of alkyl groups include, without limitation, methyl, ethyl, n-propyl, isopropyl, n-butyl, i-butyl, sec-butyl, t-butyl, amyl, t-amyl, hexyl, heptyl, octyl, nonyl, decyl, undecyl and dodecyl.

An alkyl group of the presently disclosed compounds may be substituted or unsubstituted. When substituted, the substituent group(s) can be one or more group(s) independently selected from cycloalkyl, aryl, heteroaryl, heterocycloalkyl, hydroxy, protected hydroxy, alkoxy, aryloxy, mercapto, alkylthio, arylthio, cyano, halogen, carbonyl, thiocarbonyl, O-carbamoyl, N-carbamoyl, O-thiocarbamoyl, N-thiocarbamoyl, C-amido, N-amido, S-sulfonamido, N-sulfonamido, C-carboxy, protected C-carboxy, O-carboxy, isocyanato, thiocyanato, isothiocyanato, nitro, silyl, trihalomethanesulfonyl, amino (e.g., —NRₓR_{y}) and protected amino.

As used herein, “alkenyl” refers to an alkyl group that contains in the straight or branched hydrocarbon chain one or more double bonds. An alkenyl group of the presently disclosed compounds may be unsubstituted or substituted. When substituted, the substituent(s) may be selected from the same groups disclosed above regarding alkyl group substitution, or with regard to optional substitution.

As used herein, “alkynyl” refers to an alkyl group that contains in the straight or branched hydrocarbon chain one or more triple bonds. An alkynyl group of the presently disclosed compounds may be unsubstituted or substituted. When substituted, the substituent(s) may be selected from the same groups disclosed above regarding alkyl group substitution, or with regard to optional substitution.

As used herein, “acyl” refers to an “RₓC(═O)—” group.

As used herein, “cycloalkyl” refers to a completely saturated (no double bonds) hydrocarbon ring. In some embodiments, cycloalkyl refers to a hydrocarbon ring containing no double bonds or one or more double bonds provided that they do not form a fully delocalized pi-electron system in the ring. Cycloalkyl groups of the presently disclosed compounds may range from C₃ to C₈. A cycloalkyl group may be unsubstituted or substituted. If substituted, the substituent(s) may be selected from those indicated above regarding substitution of an alkyl group. The “cycloalkyl” group can be made up of two or more fused rings (rings that share two adjacent carbon atoms). When the cycloalkyl group is a fused ring system, then the ring that is connected to the rest of the molecule is a cycloalkyl group as defined above. The other ring(s) in the fused ring system may be a cycloalkyl, a cycloalkenyl, an aryl, a heteroaryl, or a heterocycloalkyl group.

As used herein, “cycloalkenyl” refers to a cycloalkyl group that contains one or more double bonds in the ring although, if there is more than one, they cannot form a fully delocalized pi-electron system in the ring (otherwise the group would be “aryl,” as defined herein). A cycloalkenyl group of the presently disclosed compounds may unsubstituted or substituted. When substituted, the substituent(s) may be selected from the same groups disclosed above regarding alkyl group substitution. The “cycloalkenyl” group can be made up of two or more fused rings (rings that share two adjacent carbon atoms). When the cycloalkenyl group is a fused ring system, then the ring that is connected to the rest of the molecule is a cycloalkenyl group as defined above. The other ring(s) in the fused ring system may be a cycloalkyl, a cycloalkenyl, an aryl, a heteroaryl, or a heterocycloalkyl group.

The term “alkylene” refers to an alkyl group, as defined herein, which is a biradical and is connected to two other moieties. Thus, methylene (—CH₂—), ethylene (—CH₂CH₂—), propylene (—CH₂CH₂CH₂—), isopropylene (IUPAC: (methyl)ethylene) (—CH₂—CH(CH₃)—), and isobutylene (IUPAC: 2-(methyl) propylene) (—CH₂—CH(CH₃)—CH₂—) are examples, without limitation, of an alkylene group.

As used herein, “heterocycloalkyl” refers to a ring having in the ring system one or more heteroatoms independently selected from nitrogen, oxygen and sulfur. The ring may also contain one or more double bonds provided that they do not form a fully delocalized pi-electron system in the rings. The ring defined herein can be a stable 3- to 18-membered ring that consists of carbon atoms and from one to five heteroatoms selected from the group consisting of nitrogen, oxygen, and sulfur. Heterocycloalkyl groups of the presently disclosed compounds may be unsubstituted or substituted. When substituted, the substituent(s) may be one or more groups independently selected from the group consisting of halogen, hydroxy, protected hydroxy, cyano, nitro, alkyl, alkoxy, acyl, acyloxy, carboxy, protected carboxy, amino, protected amino, carboxamide, protected carboxamide, alkylsulfonamido and trifluoromethane-sulfonamido. The “heterocycloalkyl” group can be made up of two or more fused rings (rings that share two adjacent carbon atoms). When the heterocycloalkyl group is a fused ring system, then the ring that is connected to the rest of the molecule is a heterocycloalkyl group as defined above. The other ring(s) in the fused ring system may be a cycloalkyl, a cycloalkenyl, an aryl, a heteroaryl, or a heterocycloalkyl group.

As used herein, “aralkyl” refers to an alkylene substituted with an aryl group.

As used herein, “(carbocyclic)alkyl” refers to an alkylene substituted with a carbocyclic group.

As used herein, “(heterocycloalkyl)alkyl” refers to an alkylene substituted with a heterocycloalkyl group.

An “O-carboxy” group refers to a “RₓC(═O)O—” group.

A “C-carboxy” group refers to a “—C(═O)ORₓ” group.

An “acetyl” group refers to a CH₃C(═O)— group.

A “C-amido” group refers to a “—C(═O)NRₓR_{y}” group.

An “N-amido” group refers to a “R_{y}C(═O)NRₓ—” group.

Any unsubstituted or monosubstituted amine group on a compound herein can be converted to an amide, any hydroxy group can be converted to an ester and any carboxyl group can be converted to either an amide or ester using techniques well-known to those skilled in the art (see, for example Wuts, above).

It is understood that, in any compound of the presently disclosed compounds having one or more chiral centers, if an absolute stereochemistry is not expressly indicated, then each center may independently be R or S or a mixture thereof. In addition, it is understood that, in any compound of the presently disclosed compounds having one or more double bond(s) generating geometrical isomers that can be defined as E or Z each double bond may independently be E or Z, or a mixture thereof.

It is understood that the disclosure of a compound herein inherently includes the disclosure of a tautomer thereof, if applicable. For instance, the disclosure of:

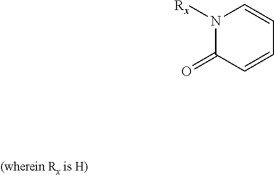

also includes the disclosure of:

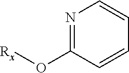

and vice versa, even if only one of the two structures is disclosed.

Throughout the present disclosure, when a compound is illustrated or named, it is understood that the isotopically enriched analogs of the compound are also contemplated. For example, a compound may have a deuterium incorporated instead of a hydrogen, or a carbon-13 instead of carbon with natural isotopic distribution. The isotopic enrichment may be in one location on the compound, i.e., only one hydrogen is replaced by a deuterium, or in more than one location. The present disclosure also encompasses compounds where all the similar atoms are replaced by their less common isotope, for example, a perdeutero compound where all the hydrogen atoms are replaced by a deuterium. The isotopically enriched compounds are useful when obtaining NMR spectra or when making use of an isotope effect in managing the kinetics of the reaction the compound undergoing.

The term “pharmaceutical composition” refers to a mixture of one or more compounds disclosed herein with one or more pharmaceutically acceptable excipients, such as, but not limited to, one or more surfactants, co-solvents, or a combination thereof. The pharmaceutical composition facilitates administration of the compound to an organism. The pharmaceutical compositions described herein are orally administered.

The term “pharmaceutically acceptable” defines an excipient or a salt that does not abrogate the biological activity and properties of the compound.

The term “therapeutically effective amount” refers to an amount of compound effective to prevent, alleviate, or ameliorate symptoms of disease or prolong the survival of the subject being treated. In some embodiments, a therapeutically effective amount means an amount of compound effective to alleviate or ameliorate symptoms of disease or prolong the survival of the subject being treated.

### Pharmaceutical Compositions

In one aspect, provided herein are pharmaceutical compositions comprising, consisting essentially of, or consisting of a compound of Formula I:

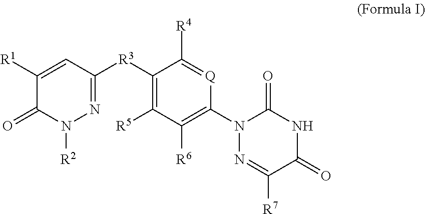

or a stereoisomer or a tautomer thereof, or a pharmaceutically acceptable salt thereof, and one or more surfactants;
wherein
R¹ is C₁-C₆ alkyl; C₂-C₁₀ alkenyl; a non-aromatic C₃-C₁₂ carbocyclic ring; a C₆-C₁₀ aryl group; a 3- to 6-membered heterocycloalkyl ring containing one to four ring heteroatoms independently selected from oxygen, sulfur, and nitrogen; a five- to ten-membered heteroaryl ring containing one to four ring heteroatoms independently selected from oxygen, sulfur, and nitrogen; a (carbocyclic)alkyl group; an aralkyl group; or a (heterocycloalkyl)alkyl group; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen;
R² is hydrogen or an optionally substituted C₁-C₆ alkyl;
R³ is independently —(C(R^{a})₂)_{z}—; oxygen; sulfur; or —NR^{a}—; wherein:
each R^{a} is independently a hydrogen or C₁-C₆ alkyl; and
z is 0, 1, 2, 3, 4 or 5;
each of R⁴ and R⁵ is independently selected from halogen, —CN, optionally substituted C₁-C₆ alkyl, optionally substituted C₁-C₆ alkoxy, optionally substituted C₂-C₆ alkenyl, or cyclopropyl;
R⁶ is selected from hydrogen, optionally substituted C₁-C₆ alkyl, optionally substituted C₁-C₆ alkoxy, cyano, or halogen;
optionally R⁵ and R⁶ taken together, along with the carbon atoms to which they are attached, form a 4-, 5- or 6-membered non-aromatic carbocyclic, heterocycloalkyl, aryl, or heteroaryl ring;
Q is nitrogen or —CR^{c}—, wherein R^{c} is hydrogen, halogen, or C₁-C₆ alkyl;
R⁷ is —(C(R^{d})₂)ₙ—N(R^{d})₂; wherein
each R^{d} is independently hydrogen or optionally substituted C₁-C₆ alkyl; and
each n is independently selected from 0 or 1; and
   wherein the composition is formulated for oral administration.

In some embodiments, R¹ is C₁-C₆ alkyl, C₂-C₁₀ alkenyl, cyclopropyl, cyclobutyl, cyclopentyl, cyclohexyl, tetrahydrofuranyl, tetrahydropyranyl, azetidinyl, pyridazin-3(2H)-one, phenyl, naphthyl, pyridinyl, cinnolinyl, isoquinolinyl, quinolinyl, pyrazolo[1,5-a]pyridinyl, imidazo[1,5-a]pyridinyl, benzo[b]thiophenyl, a (cyclobutyl)alkyl group, a (cyclopentyl)alkyl group, a benzyl group, a (tetrahydrofuranyl)alkyl group, or a (tetrahydropyranyl)alkyl group; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen.

In some embodiments, R¹ is a C₆-C₁₀ aryl group or a five- to ten-membered heteroaryl ring containing one to four ring heteroatoms independently selected from oxygen, sulfur, and nitrogen; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen.

In some embodiments, R¹ is benzene optionally substituted with one to five R^{g} independently selected from the group consisting of hydroxy, halogen, CN, C₁-C₆ alkyl, C₁-C₆ alkoxy, and C₁-C₆ haloalkoxy, or two R^{g} together with the atoms to which they are attached form a ring, or two R^{g} together with the atoms to which they are attached form an aromatic or non-aromatic 3- to 6-membered ring, optionally containing one or two ring heteroatoms independently selected from oxygen, sulfur, and nitrogen.

In some embodiments, R¹ is C₁-C₆ alkyl or a non-aromatic C₃-C₁₂ carbocyclic ring; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy. In some embodiments, R¹ is C₁-C₆ alkyl optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy. In some embodiments, R¹ is a non-aromatic C₃-C₁₂ carbocyclic ring optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy.

In some embodiments, R³ is oxygen or —CH₂—.

In some embodiments, Q is —CH—.

In some embodiments, each of R⁴ and R⁵ is independently halogen or C₁-C₈alkyl optionally substituted with one to five substituents independently selected from hydroxy, halogen, and C₁-C₆ alkoxy; and R⁶ is hydrogen; or R⁴ is halogen or C₁-C₈alkyl optionally substituted with one to five substituents independently selected from hydroxy, halogen, and C₁-C₆ alkoxy; and R⁵ and R⁶ taken together, along with the carbon atoms to which they are attached, form a 4-, 5- or 6-membered carbocyclic ring.

In some embodiments, the compound is selected from the group consisting of:

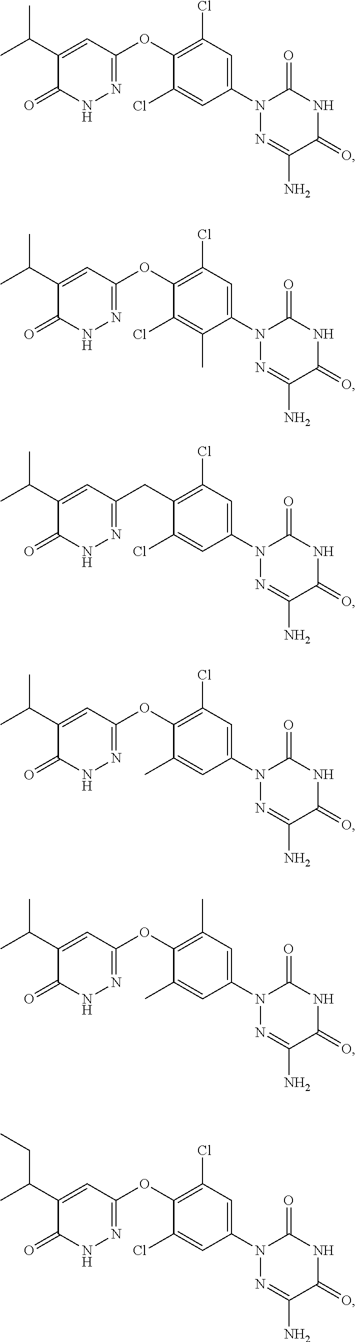 [SMILES: CC(C)c1cc(Oc2c(Cl)cc(-n3nc(N)c(=O)[nH]c3=O)cc2Cl)n[nH]c1=O ; Cc1c(-n2nc(N)c(=O)[nH]c2=O)cc(Cl)c(Oc2cc(C(C)C)c(=O)[nH]n2)c1Cl ; CC(C)c1cc(Cc2c(Cl)cc(-n3nc(N)c(=O)[nH]c3=O)cc2Cl)n[nH]c1=O ; Cc1cc(-n2nc(N)c(=O)[nH]c2=O)cc(Cl)c1Oc1cc(C(C)C)c(=O)[nH]n1 ; Cc1cc(-n2nc(N)c(=O)[nH]c2=O)cc(C)c1Oc1cc(C(C)C)c(=O)[nH]n1 ; CCC(C)c1cc(Oc2c(Cl)cc(-n3nc(N)c(=O)[nH]c3=O)cc2Cl)n[nH]c1=O]

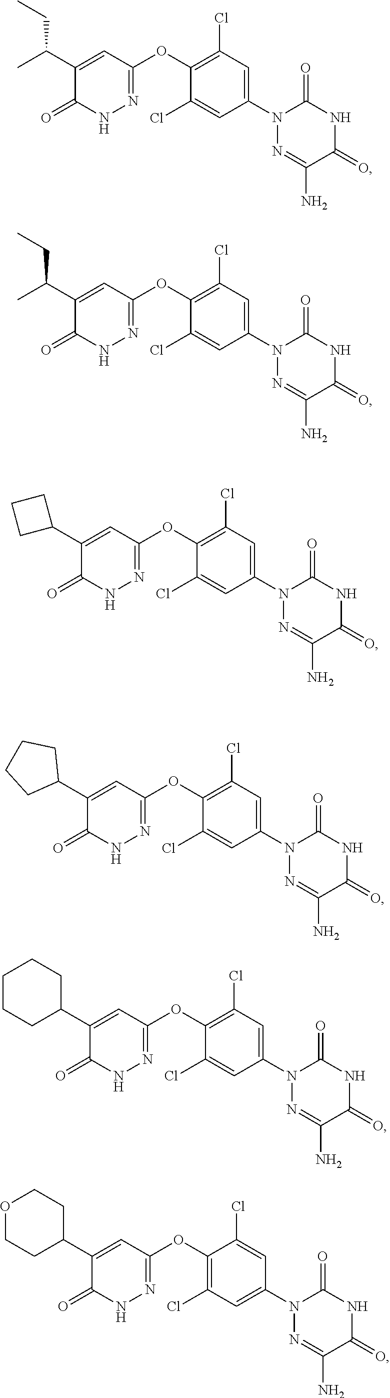 [SMILES: CC[C@@H](C)c1cc(Oc2c(Cl)cc(-n3nc(N)c(=O)[nH]c3=O)cc2Cl)n[nH]c1=O ; CC[C@H](C)c1cc(Oc2c(Cl)cc(-n3nc(N)c(=O)[nH]c3=O)cc2Cl)n[nH]c1=O ; Nc1nn(-c2cc(Cl)c(Oc3cc(C4CCC4)c(=O)[nH]n3)c(Cl)c2)c(=O)[nH]c1=O ; Nc1nn(-c2cc(Cl)c(Oc3cc(C4CCCC4)c(=O)[nH]n3)c(Cl)c2)c(=O)[nH]c1=O ; Nc1nn(-c2cc(Cl)c(Oc3cc(C4CCCCC4)c(=O)[nH]n3)c(Cl)c2)c(=O)[nH]c1=O ; Nc1nn(-c2cc(Cl)c(Oc3cc(C4CCOCC4)c(=O)[nH]n3)c(Cl)c2)c(=O)[nH]c1=O]

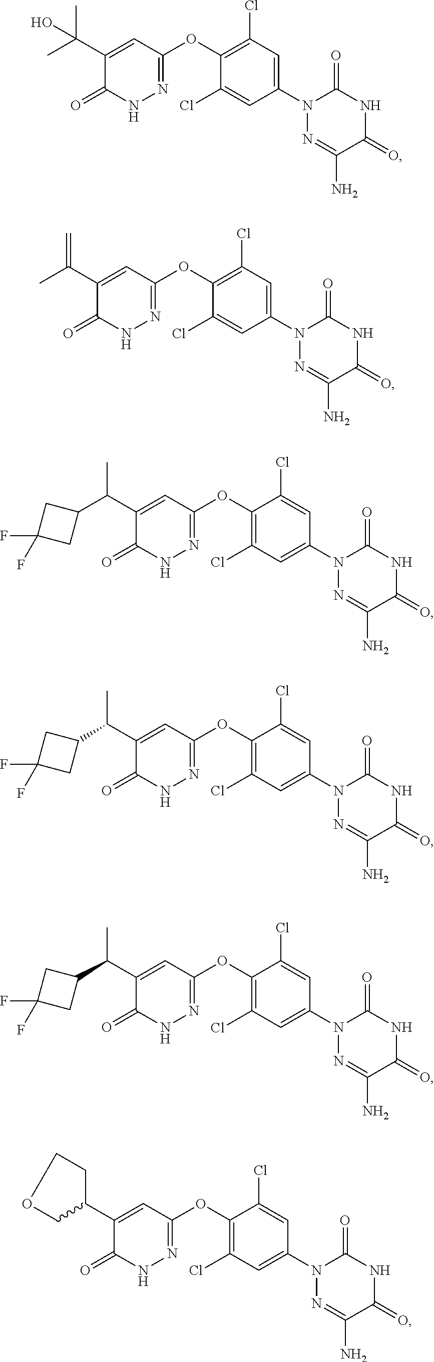 [SMILES: CC(C)(O)c1cc(Oc2c(Cl)cc(-n3nc(N)c(=O)[nH]c3=O)cc2Cl)n[nH]c1=O ; C=C(C)c1cc(Oc2c(Cl)cc(-n3nc(N)c(=O)[nH]c3=O)cc2Cl)n[nH]c1=O ; CC(c1cc(Oc2c(Cl)cc(-n3nc(N)c(=O)[nH]c3=O)cc2Cl)n[nH]c1=O)C1CC(F)(F)C1 ; C[C@H](c1cc(Oc2c(Cl)cc(-n3nc(N)c(=O)[nH]c3=O)cc2Cl)n[nH]c1=O)C1CC(F)(F)C1 ; C[C@@H](c1cc(Oc2c(Cl)cc(-n3nc(N)c(=O)[nH]c3=O)cc2Cl)n[nH]c1=O)C1CC(F)(F)C1 ; Nc1nn(-c2cc(Cl)c(Oc3cc(C4CCOC4)c(=O)[nH]n3)c(Cl)c2)c(=O)[nH]c1=O]

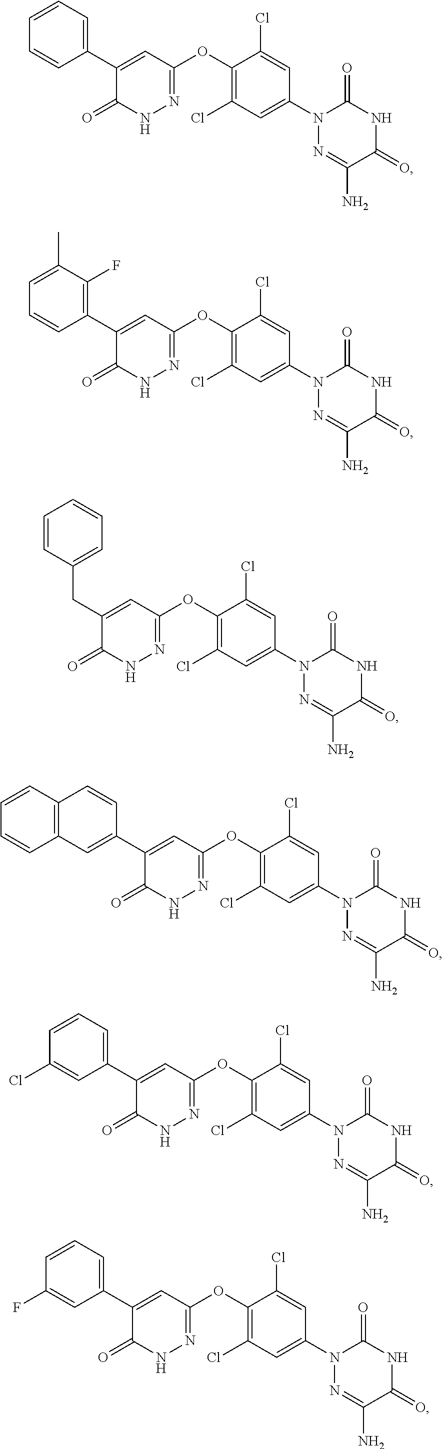 [SMILES: Nc1nn(-c2cc(Cl)c(Oc3cc(-c4ccccc4)c(=O)[nH]n3)c(Cl)c2)c(=O)[nH]c1=O ; Cc1cccc(-c2cc(Oc3c(Cl)cc(-n4nc(N)c(=O)[nH]c4=O)cc3Cl)n[nH]c2=O)c1F ; Nc1nn(-c2cc(Cl)c(Oc3cc(Cc4ccccc4)c(=O)[nH]n3)c(Cl)c2)c(=O)[nH]c1=O ; Nc1nn(-c2cc(Cl)c(Oc3cc(-c4ccc5ccccc5c4)c(=O)[nH]n3)c(Cl)c2)c(=O)[nH]c1=O ; Nc1nn(-c2cc(Cl)c(Oc3cc(-c4cccc(Cl)c4)c(=O)[nH]n3)c(Cl)c2)c(=O)[nH]c1=O ; Nc1nn(-c2cc(Cl)c(Oc3cc(-c4cccc(F)c4)c(=O)[nH]n3)c(Cl)c2)c(=O)[nH]c1=O]

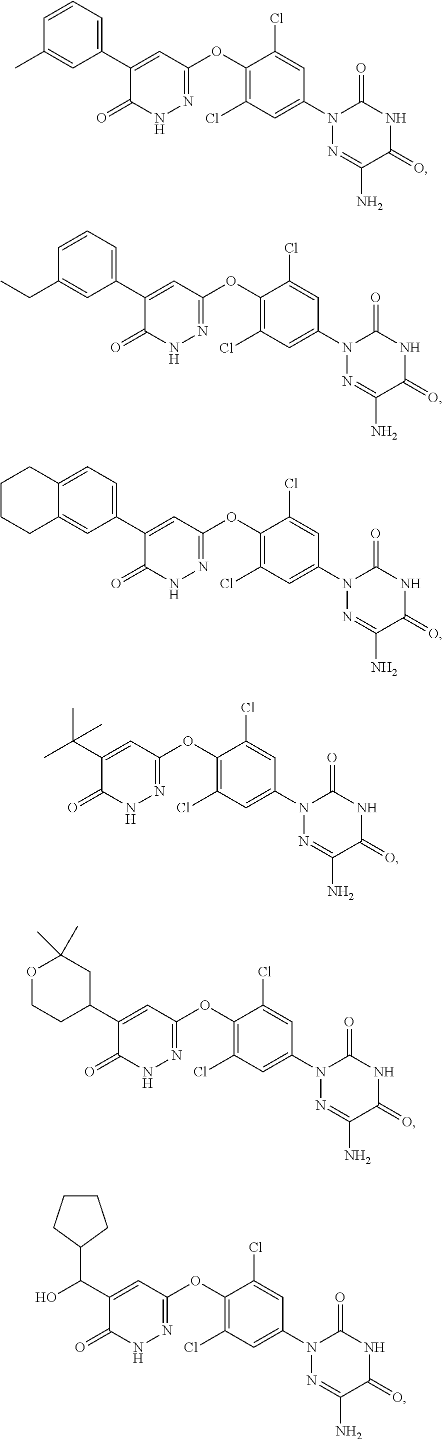 [SMILES: Cc1cccc(-c2cc(Oc3c(Cl)cc(-n4nc(N)c(=O)[nH]c4=O)cc3Cl)n[nH]c2=O)c1 ; CCc1cccc(-c2cc(Oc3c(Cl)cc(-n4nc(N)c(=O)[nH]c4=O)cc3Cl)n[nH]c2=O)c1 ; Nc1nn(-c2cc(Cl)c(Oc3cc(-c4ccc5c(c4)CCCC5)c(=O)[nH]n3)c(Cl)c2)c(=O)[nH]c1=O ; CC(C)(C)c1cc(Oc2c(Cl)cc(-n3nc(N)c(=O)[nH]c3=O)cc2Cl)n[nH]c1=O ; CC1(C)CC(c2cc(Oc3c(Cl)cc(-n4nc(N)c(=O)[nH]c4=O)cc3Cl)n[nH]c2=O)CCO1 ; Nc1nn(-c2cc(Cl)c(Oc3cc(C(O)C4CCCC4)c(=O)[nH]n3)c(Cl)c2)c(=O)[nH]c1=O]

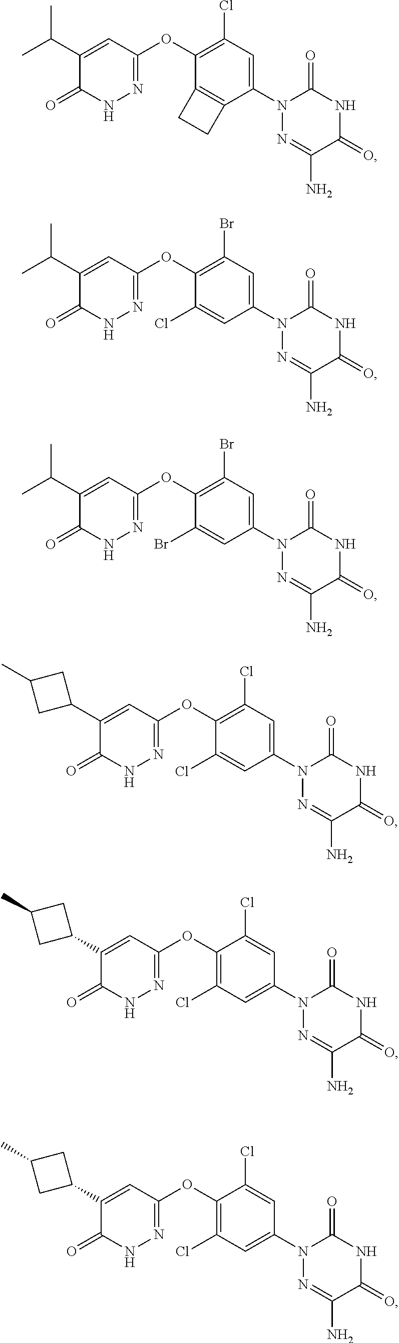 [SMILES: CC(C)c1cc(Oc2c(Cl)cc(-n3nc(N)c(=O)[nH]c3=O)c3c2CC3)n[nH]c1=O ; CC(C)c1cc(Oc2c(Cl)cc(-n3nc(N)c(=O)[nH]c3=O)cc2Br)n[nH]c1=O ; CC(C)c1cc(Oc2c(Br)cc(-n3nc(N)c(=O)[nH]c3=O)cc2Br)n[nH]c1=O ; CC1CC(c2cc(Oc3c(Cl)cc(-n4nc(N)c(=O)[nH]c4=O)cc3Cl)n[nH]c2=O)C1 ; C[C@H]1C[C@H](c2cc(Oc3c(Cl)cc(-n4nc(N)c(=O)[nH]c4=O)cc3Cl)n[nH]c2=O)C1 ; C[C@H]1C[C@@H](c2cc(Oc3c(Cl)cc(-n4nc(N)c(=O)[nH]c4=O)cc3Cl)n[nH]c2=O)C1]

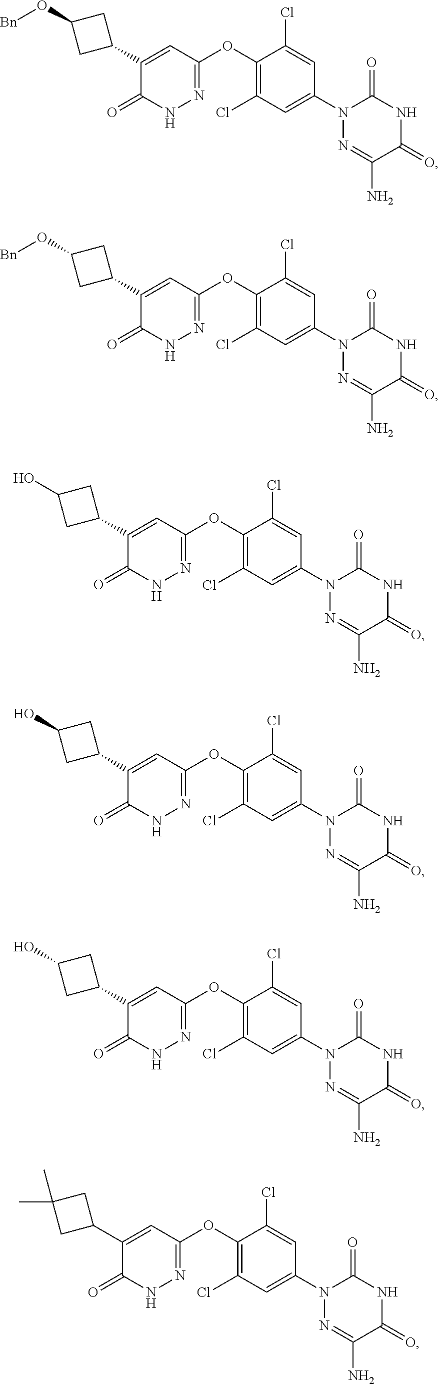

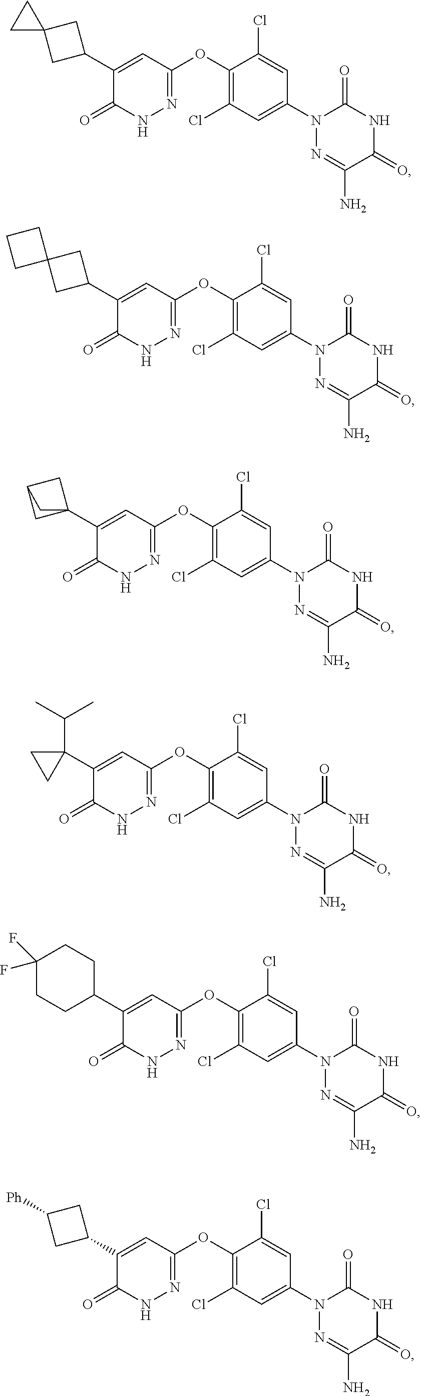 [SMILES: Nc1nn(-c2cc(Cl)c(Oc3cc(C4CC5(CC5)C4)c(=O)[nH]n3)c(Cl)c2)c(=O)[nH]c1=O ; Nc1nn(-c2cc(Cl)c(Oc3cc(C4CC5(CCC5)C4)c(=O)[nH]n3)c(Cl)c2)c(=O)[nH]c1=O ; Nc1nn(-c2cc(Cl)c(Oc3cc(C45CC(C4)C5)c(=O)[nH]n3)c(Cl)c2)c(=O)[nH]c1=O ; CC(C)C1(c2cc(Oc3c(Cl)cc(-n4nc(N)c(=O)[nH]c4=O)cc3Cl)n[nH]c2=O)CC1 ; Nc1nn(-c2cc(Cl)c(Oc3cc(C4CCC(F)(F)CC4)c(=O)[nH]n3)c(Cl)c2)c(=O)[nH]c1=O ; Nc1nn(-c2cc(Cl)c(Oc3cc([C@H]4C[C@@H](c5ccccc5)C4)c(=O)[nH]n3)c(Cl)c2)c(=O)[nH]c1=O]

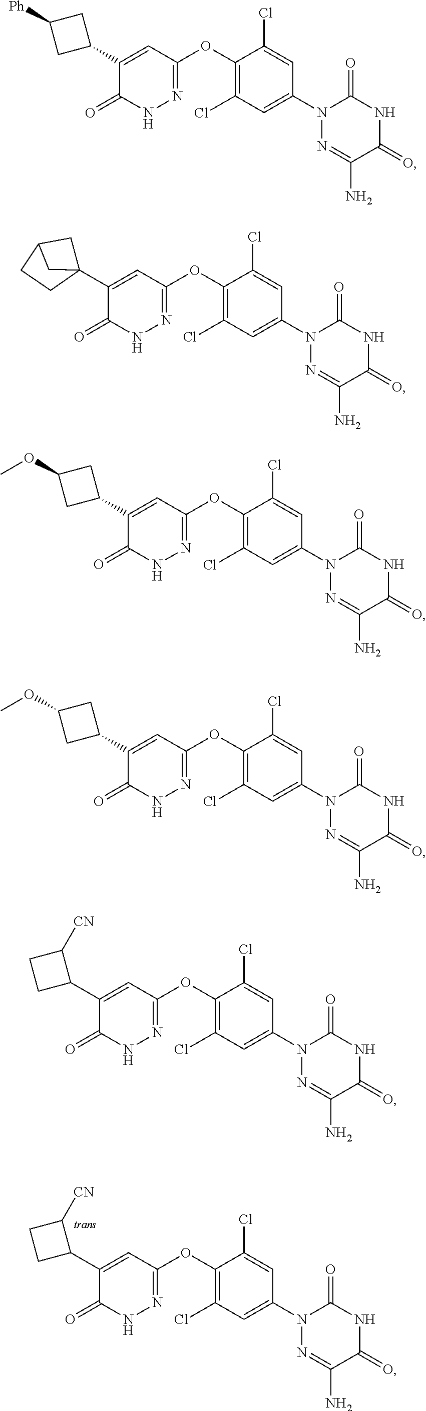 [SMILES: Nc1nn(-c2cc(Cl)c(Oc3cc([C@H]4C[C@H](c5ccccc5)C4)c(=O)[nH]n3)c(Cl)c2)c(=O)[nH]c1=O ; Nc1nn(-c2cc(Cl)c(Oc3cc(C45CCC(C4)C5)c(=O)[nH]n3)c(Cl)c2)c(=O)[nH]c1=O ; CO[C@H]1C[C@H](c2cc(Oc3c(Cl)cc(-n4nc(N)c(=O)[nH]c4=O)cc3Cl)n[nH]c2=O)C1 ; CO[C@H]1C[C@@H](c2cc(Oc3c(Cl)cc(-n4nc(N)c(=O)[nH]c4=O)cc3Cl)n[nH]c2=O)C1 ; N#CC1CCC1c1cc(Oc2c(Cl)cc(-n3nc(N)c(=O)[nH]c3=O)cc2Cl)n[nH]c1=O ; N#CC1CCC1c1cc(Oc2c(Cl)cc(-n3nc(N)c(=O)[nH]c3=O)cc2Cl)n[nH]c1=O]

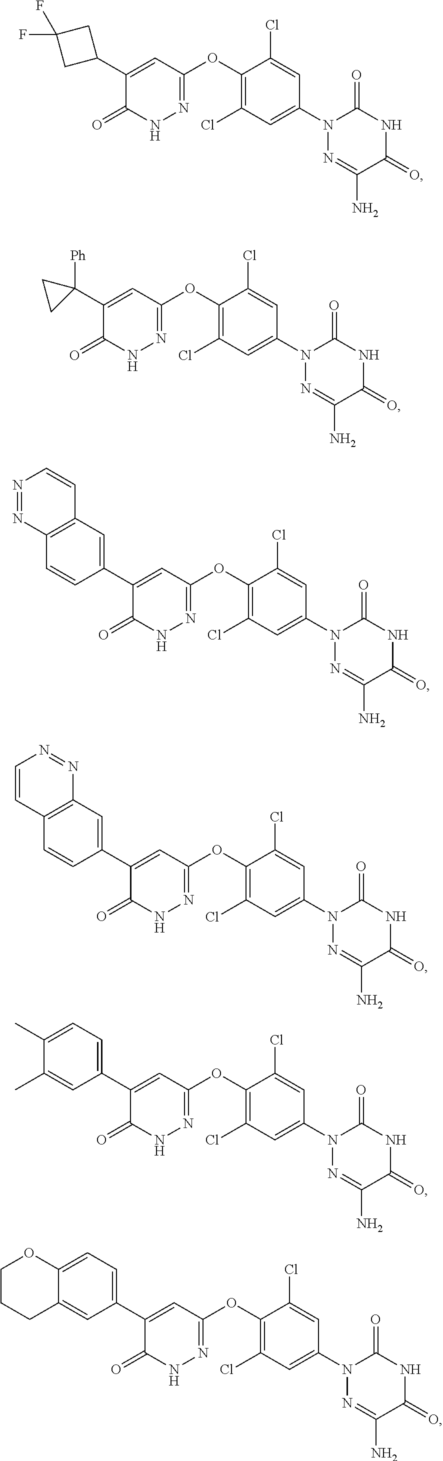 [SMILES: Nc1nn(-c2cc(Cl)c(Oc3cc(C4CC(F)(F)C4)c(=O)[nH]n3)c(Cl)c2)c(=O)[nH]c1=O ; Nc1nn(-c2cc(Cl)c(Oc3cc(C4(c5ccccc5)CC4)c(=O)[nH]n3)c(Cl)c2)c(=O)[nH]c1=O ; Nc1nn(-c2cc(Cl)c(Oc3cc(-c4ccc5nnccc5c4)c(=O)[nH]n3)c(Cl)c2)c(=O)[nH]c1=O ; Nc1nn(-c2cc(Cl)c(Oc3cc(-c4ccc5ccnnc5c4)c(=O)[nH]n3)c(Cl)c2)c(=O)[nH]c1=O ; Cc1ccc(-c2cc(Oc3c(Cl)cc(-n4nc(N)c(=O)[nH]c4=O)cc3Cl)n[nH]c2=O)cc1C ; Nc1nn(-c2cc(Cl)c(Oc3cc(-c4ccc5c(c4)CCCO5)c(=O)[nH]n3)c(Cl)c2)c(=O)[nH]c1=O]

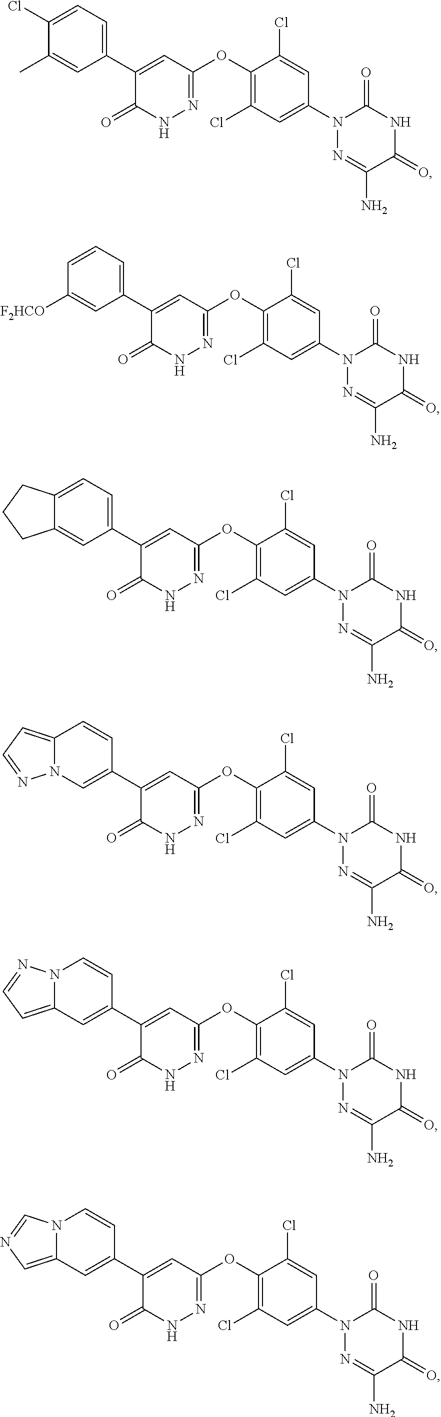 [SMILES: Cc1cc(-c2cc(Oc3c(Cl)cc(-n4nc(N)c(=O)[nH]c4=O)cc3Cl)n[nH]c2=O)ccc1Cl ; Nc1nn(-c2cc(Cl)c(Oc3cc(-c4cccc(OC(F)F)c4)c(=O)[nH]n3)c(Cl)c2)c(=O)[nH]c1=O ; Nc1nn(-c2cc(Cl)c(Oc3cc(-c4ccc5c(c4)CCC5)c(=O)[nH]n3)c(Cl)c2)c(=O)[nH]c1=O ; Nc1nn(-c2cc(Cl)c(Oc3cc(-c4ccc5ccnn5c4)c(=O)[nH]n3)c(Cl)c2)c(=O)[nH]c1=O ; Nc1nn(-c2cc(Cl)c(Oc3cc(-c4ccn5nccc5c4)c(=O)[nH]n3)c(Cl)c2)c(=O)[nH]c1=O ; Nc1nn(-c2cc(Cl)c(Oc3cc(-c4ccn5cncc5c4)c(=O)[nH]n3)c(Cl)c2)c(=O)[nH]c1=O]

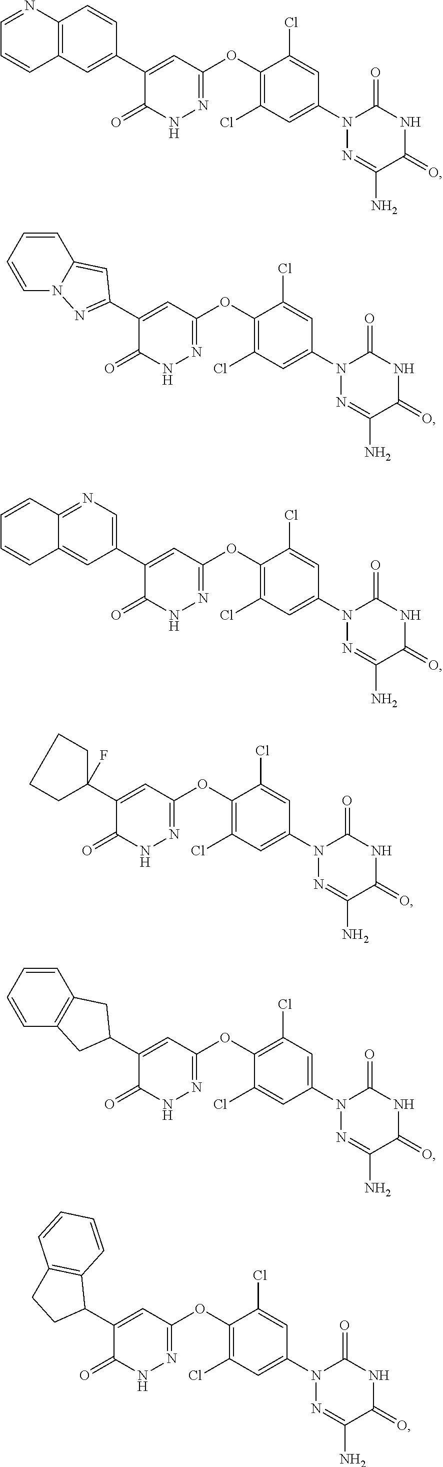 [SMILES: Nc1nn(-c2cc(Cl)c(Oc3cc(-c4ccc5ncccc5c4)c(=O)[nH]n3)c(Cl)c2)c(=O)[nH]c1=O ; Nc1nn(-c2cc(Cl)c(Oc3cc(-c4cc5ccccn5n4)c(=O)[nH]n3)c(Cl)c2)c(=O)[nH]c1=O ; Nc1nn(-c2cc(Cl)c(Oc3cc(-c4cnc5ccccc5c4)c(=O)[nH]n3)c(Cl)c2)c(=O)[nH]c1=O ; Nc1nn(-c2cc(Cl)c(Oc3cc(C4(F)CCCC4)c(=O)[nH]n3)c(Cl)c2)c(=O)[nH]c1=O ; Nc1nn(-c2cc(Cl)c(Oc3cc(C4Cc5ccccc5C4)c(=O)[nH]n3)c(Cl)c2)c(=O)[nH]c1=O ; Nc1nn(-c2cc(Cl)c(Oc3cc(C4CCc5ccccc54)c(=O)[nH]n3)c(Cl)c2)c(=O)[nH]c1=O]

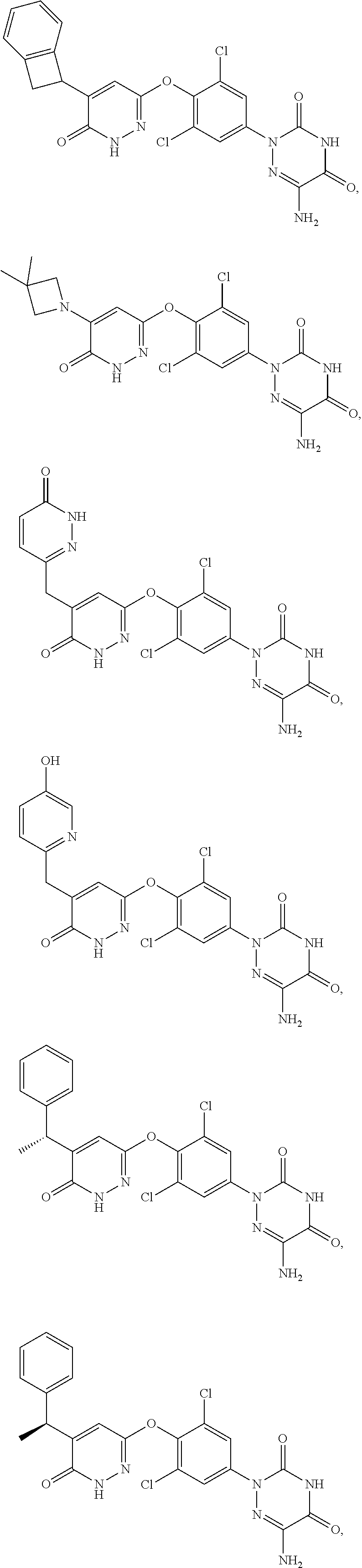 [SMILES: Nc1nn(-c2cc(Cl)c(Oc3cc(C4Cc5ccccc54)c(=O)[nH]n3)c(Cl)c2)c(=O)[nH]c1=O ; CC1(C)CN(c2cc(Oc3c(Cl)cc(-n4nc(N)c(=O)[nH]c4=O)cc3Cl)n[nH]c2=O)C1 ; Nc1nn(-c2cc(Cl)c(Oc3cc(Cc4ccc(=O)[nH]n4)c(=O)[nH]n3)c(Cl)c2)c(=O)[nH]c1=O ; Nc1nn(-c2cc(Cl)c(Oc3cc(Cc4ccc(O)cn4)c(=O)[nH]n3)c(Cl)c2)c(=O)[nH]c1=O ; C[C@H](c1ccccc1)c1cc(Oc2c(Cl)cc(-n3nc(N)c(=O)[nH]c3=O)cc2Cl)n[nH]c1=O ; C[C@@H](c1ccccc1)c1cc(Oc2c(Cl)cc(-n3nc(N)c(=O)[nH]c3=O)cc2Cl)n[nH]c1=O]

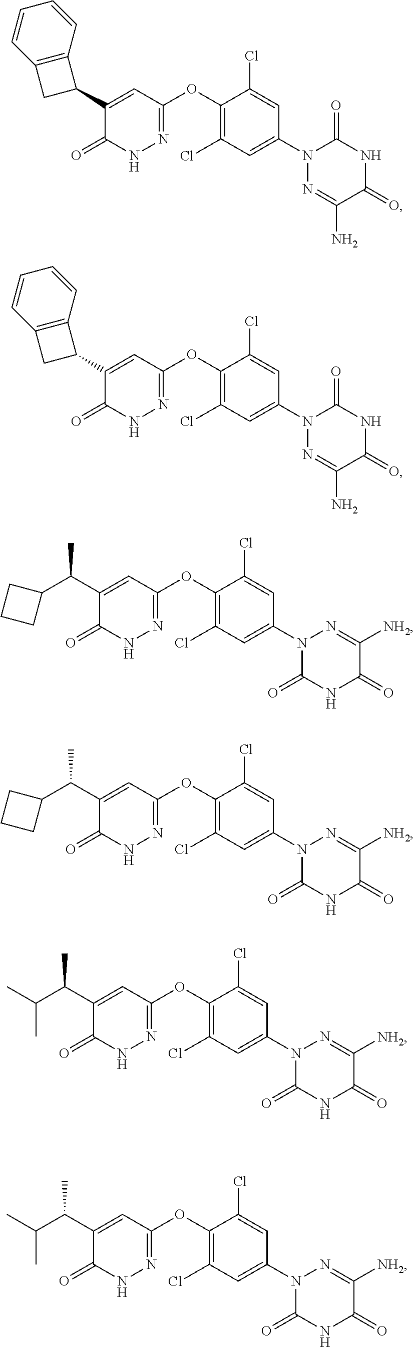 [SMILES: Nc1nn(-c2cc(Cl)c(Oc3cc([C@H]4Cc5ccccc54)c(=O)[nH]n3)c(Cl)c2)c(=O)[nH]c1=O ; Nc1nn(-c2cc(Cl)c(Oc3cc([C@@H]4Cc5ccccc54)c(=O)[nH]n3)c(Cl)c2)c(=O)[nH]c1=O ; C[C@@H](c1cc(Oc2c(Cl)cc(-n3nc(N)c(=O)[nH]c3=O)cc2Cl)n[nH]c1=O)C1CCC1 ; C[C@H](c1cc(Oc2c(Cl)cc(-n3nc(N)c(=O)[nH]c3=O)cc2Cl)n[nH]c1=O)C1CCC1 ; CC(C)[C@@H](C)c1cc(Oc2c(Cl)cc(-n3nc(N)c(=O)[nH]c3=O)cc2Cl)n[nH]c1=O ; CC(C)[C@H](C)c1cc(Oc2c(Cl)cc(-n3nc(N)c(=O)[nH]c3=O)cc2Cl)n[nH]c1=O]

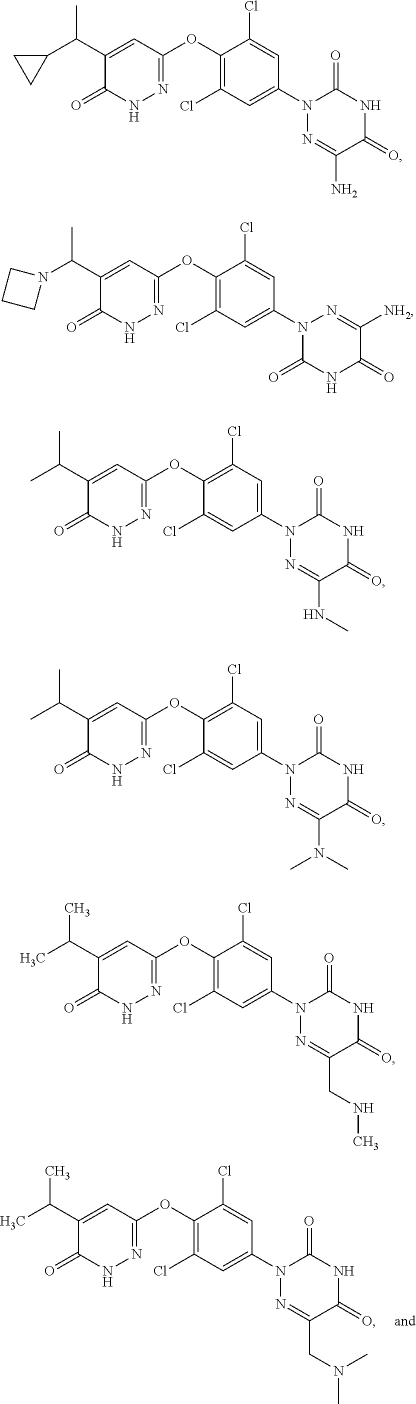

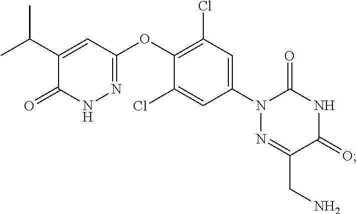 [SMILES: CC(C)c1cc(Oc2c(Cl)cc(-n3nc(CN)c(=O)[nH]c3=O)cc2Cl)n[nH]c1=O]

or a stereoisomer or a tautomer thereof, or a pharmaceutically acceptable salt thereof.

In some embodiments, R¹ is unsubstituted C₁-C₆ alkyl or unsubstituted C₃-C₆ carbocyclic ring; R² is hydrogen; R³ is —CH₂— or oxygen; Q is —CH—; R⁷ is NH₂ and (i) each of R⁴ and R⁵ is independently selected from halogen and C₁-C₆ alkyl, and R⁶ is hydrogen or C₁-C₆ alkyl, or (ii) R⁴ is halogen, and R⁵ and R⁶ taken together, along with the carbon atoms to which they are attached, form a 4-, 5- or 6-membered non-aromatic carbocyclic ring. In some embodiments, the compound is selected from the group consisting of:

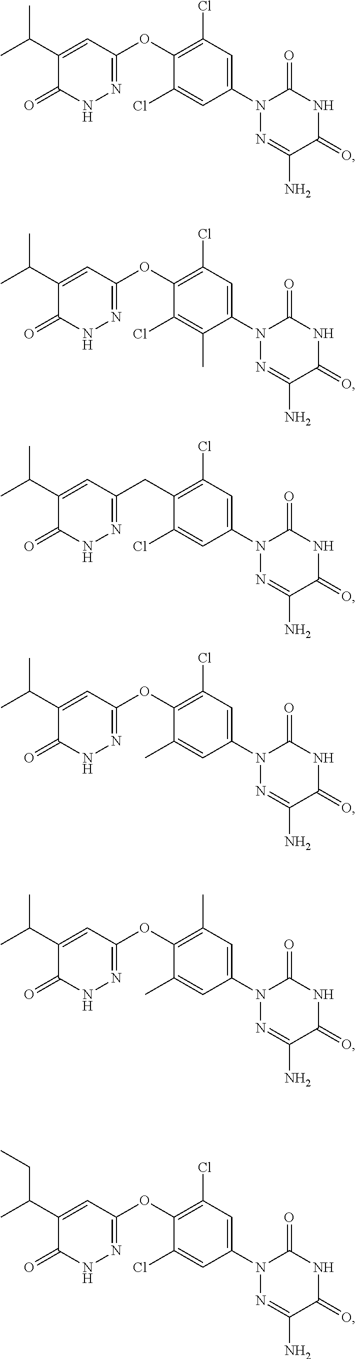 [SMILES: CC(C)c1cc(Oc2c(Cl)cc(-n3nc(N)c(=O)[nH]c3=O)cc2Cl)n[nH]c1=O ; Cc1c(-n2nc(N)c(=O)[nH]c2=O)cc(Cl)c(Oc2cc(C(C)C)c(=O)[nH]n2)c1Cl ; CC(C)c1cc(Cc2c(Cl)cc(-n3nc(N)c(=O)[nH]c3=O)cc2Cl)n[nH]c1=O ; Cc1cc(-n2nc(N)c(=O)[nH]c2=O)cc(Cl)c1Oc1cc(C(C)C)c(=O)[nH]n1 ; Cc1cc(-n2nc(N)c(=O)[nH]c2=O)cc(C)c1Oc1cc(C(C)C)c(=O)[nH]n1 ; CCC(C)c1cc(Oc2c(Cl)cc(-n3nc(N)c(=O)[nH]c3=O)cc2Cl)n[nH]c1=O]

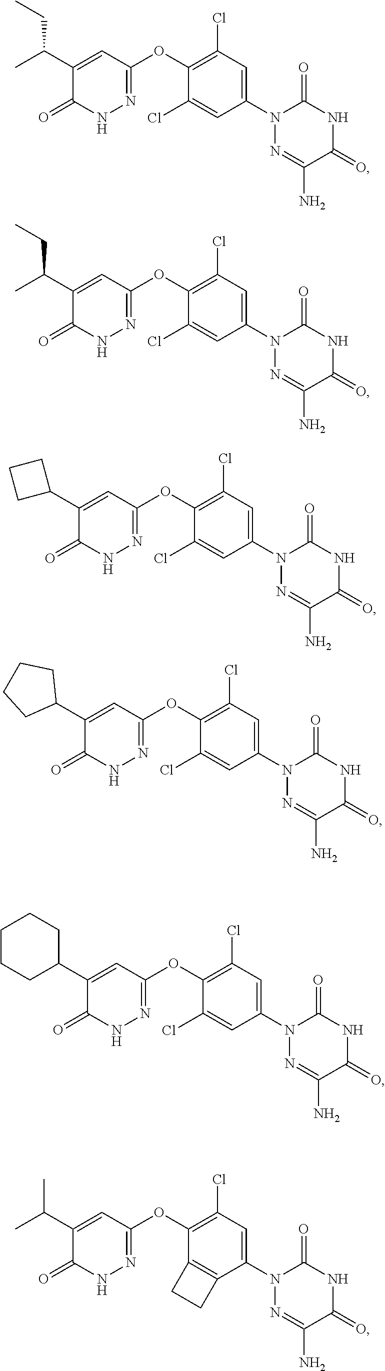 [SMILES: CC[C@@H](C)c1cc(Oc2c(Cl)cc(-n3nc(N)c(=O)[nH]c3=O)cc2Cl)n[nH]c1=O ; CC[C@H](C)c1cc(Oc2c(Cl)cc(-n3nc(N)c(=O)[nH]c3=O)cc2Cl)n[nH]c1=O ; Nc1nn(-c2cc(Cl)c(Oc3cc(C4CCC4)c(=O)[nH]n3)c(Cl)c2)c(=O)[nH]c1=O ; Nc1nn(-c2cc(Cl)c(Oc3cc(C4CCCC4)c(=O)[nH]n3)c(Cl)c2)c(=O)[nH]c1=O ; Nc1nn(-c2cc(Cl)c(Oc3cc(C4CCCCC4)c(=O)[nH]n3)c(Cl)c2)c(=O)[nH]c1=O ; CC(C)c1cc(Oc2c(Cl)cc(-n3nc(N)c(=O)[nH]c3=O)c3c2CC3)n[nH]c1=O]

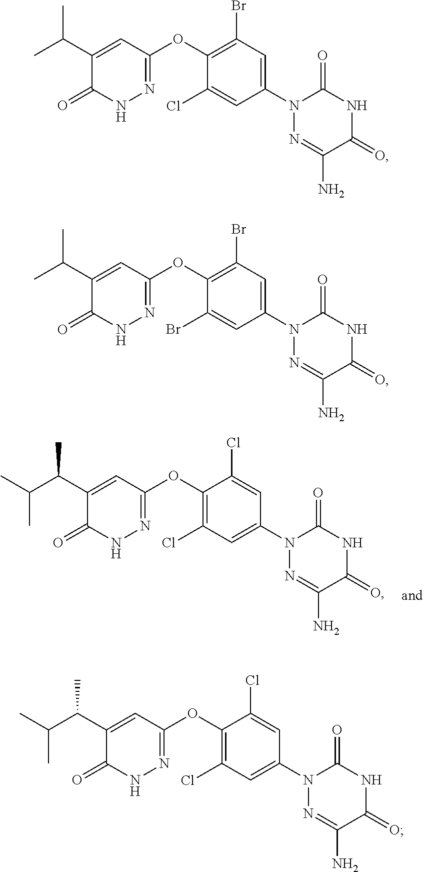 [SMILES: CC(C)c1cc(Oc2c(Cl)cc(-n3nc(N)c(=O)[nH]c3=O)cc2Br)n[nH]c1=O ; CC(C)c1cc(Oc2c(Br)cc(-n3nc(N)c(=O)[nH]c3=O)cc2Br)n[nH]c1=O ; CC(C)[C@@H](C)c1cc(Oc2c(Cl)cc(-n3nc(N)c(=O)[nH]c3=O)cc2Cl)n[nH]c1=O ; CC(C)[C@H](C)c1cc(Oc2c(Cl)cc(-n3nc(N)c(=O)[nH]c3=O)cc2Cl)n[nH]c1=O]

or a stereoisomer or a tautomer thereof, or a pharmaceutically acceptable salt thereof.

In some embodiments, the compound is

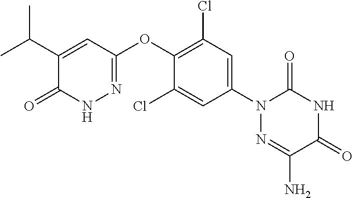 [SMILES: CC(C)c1cc(Oc2c(Cl)cc(-n3nc(N)c(=O)[nH]c3=O)cc2Cl)n[nH]c1=O]

or a stereoisomer or a tautomer thereof, or a pharmaceutically acceptable salt thereof. In some embodiments, the compound is

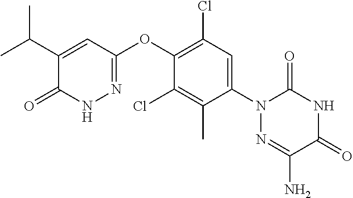 [SMILES: Cc1c(-n2nc(N)c(=O)[nH]c2=O)cc(Cl)c(Oc2cc(C(C)C)c(=O)[nH]n2)c1Cl]

or a stereoisomer or a tautomer thereof, or a pharmaceutically acceptable salt thereof. In some embodiments, the compound is

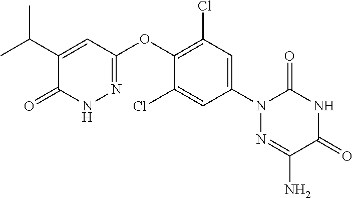 [SMILES: CC(C)c1cc(Oc2c(Cl)cc(-n3nc(N)c(=O)[nH]c3=O)cc2Cl)n[nH]c1=O]

or a stereoisomer or a tautomer thereof, or a pharmaceutically acceptable salt thereof. In some embodiments, the compound is

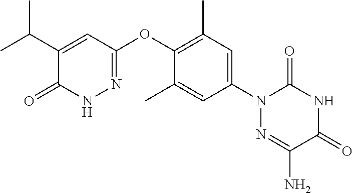 [SMILES: Cc1cc(-n2nc(N)c(=O)[nH]c2=O)cc(C)c1Oc1cc(C(C)C)c(=O)[nH]n1]

or a stereoisomer or a tautomer thereof, or a pharmaceutically acceptable salt thereof. In some embodiments, the compound is

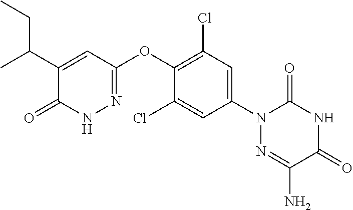 [SMILES: CCC(C)c1cc(Oc2c(Cl)cc(-n3nc(N)c(=O)[nH]c3=O)cc2Cl)n[nH]c1=O]

or a stereoisomer or a tautomer thereof, or a pharmaceutically acceptable salt thereof. In some embodiments, the compound is

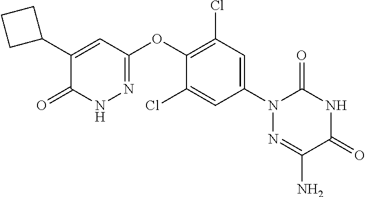 [SMILES: Nc1nn(-c2cc(Cl)c(Oc3cc(C4CCC4)c(=O)[nH]n3)c(Cl)c2)c(=O)[nH]c1=O]

or a stereoisomer or a tautomer thereof, or a pharmaceutically acceptable salt thereof. In some embodiments, the compound is In some embodiments, the compound is

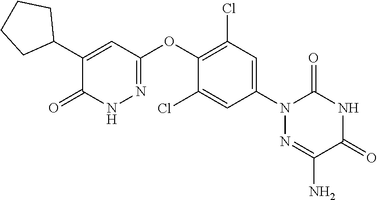 [SMILES: Nc1nn(-c2cc(Cl)c(Oc3cc(C4CCCC4)c(=O)[nH]n3)c(Cl)c2)c(=O)[nH]c1=O]

or a stereoisomer or a tautomer thereof, or a pharmaceutically acceptable salt thereof. In some embodiments, the compound is

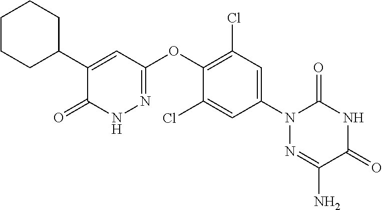 [SMILES: Nc1nn(-c2cc(Cl)c(Oc3cc(C4CCCCC4)c(=O)[nH]n3)c(Cl)c2)c(=O)[nH]c1=O]

or a stereoisomer or a tautomer thereof, or a pharmaceutically acceptable salt thereof.

In some embodiments, the compound is present in the composition in an amount of about 0.025 wt. % to about 1.0 wt. %. This includes about 0.025, 0.030, 0.035, 0.040, 0.045, 0.050, 0.055, 0.060, 0.065, 0.070, 0.075, 0.080, 0.085, 0.090, 0.095, 0.100, 0.105, 0.11, 0.115, 0.12, 0.125, 0.13, 0.135, 0.14, 0.145, 0.15, 0.155, 0.16, 0.165, 0.17, 0.175, 0.18, 0.185, 0.19, 0.195, 0.20, 0.205, 0.21, 0.215, 0.22, 0.225, 0.23, 0.235, 0.24, 0.245, 0.25, 0.255, 0.26, 0.265, 0.27, 0.275, 0.28, 0.285, 0.29, 0.295, 0.30, 0.305, 0.31, 0.315, 0.32, 0.325, 0.33, 0.335, 0.34, 0.345, 0.35, 0.355, 0.36, 0.365, 0.37, 0.375, 0.38, 0.385, 0.39, 0.395, 0.40, 0.405, 0.41, 0.415, 0.42, 0.425, 0.43, 0.435, 0.44, 0.445, 0.45, 0.50, 0.55, 0.60, 0.65, 0.70, 0.75, 0.80, 0.85, 0.90, 0.95, or 1.0 wt. %, including any value therebetween. In some embodiments, the compound is present in the composition in an amount of about 0.025 wt. % to about 0.45 wt. %. In some embodiments, the compound is present in the composition in an amount of about 0.025 wt. % to about 0.3 wt. %, about 0.025 wt. % to about 0.25 wt. %, about 0.025 wt. % to about 0.2 wt. %, about 0.05 wt. % to about 0.4 wt. %, about 0.05 wt. % to about 0.3 wt. %, about 0.05 wt. % to about 0.2 wt. %, about 0.1 wt. % to about 0.4 wt. %, or about 0.1 wt. % to about 0.3 wt. %.

In some embodiments, the one or more surfactants comprise, consist essentially of, or consist of a high hydrophilic-lipophilic balance (HLB) surfactant. As used herein, a “high HLB surfactant” refers to a surfactant with an HLB value of over 10. Non-limiting examples of high HLB surfactants include, but are not limited to, polysorbate 80 (e.g., TWEEN® 80), PEG 35 castor oil (e.g., KOLLIPHOR® ELP), PEG 40 hydrogenated castor oil (e.g., KOLLIPHOR® RH 40), PEG 32 lauroyl glycerides (e.g., GELUCIRE® 44/14), PEG 32 stearate (e.g., GELUCIRE® 48/16), PEG 32 stearoyl glycerides (e.g., GELUCIRE® 50/13), and D alpha tocopherol PEG 1000 succinate. Accordingly, in some embodiments, the high HLB surfactant is selected from the group consisting of polysorbate 80, PEG 35 castor oil, PEG 40 hydrogenated castor oil, PEG 32 lauroyl glycerides, PEG 32 stearate, PEG 32 stearoyl glycerides, D alpha tocopherol PEG 1000 succinate (vitamin E TPGS), and any combination of two or more thereof. In some embodiments, the high HLB surfactant is castor oil. In some embodiments, the high HLB surfactant is PEG 40 hydrogenated castor oil. In some embodiments, the high HLB surfactant is PEG 35 castor oil.

In some embodiments, the high HLB surfactant is present in the composition in an amount of about 50 wt. % to about 99.975 wt. %. This includes 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt. %, including any value therebetween. In some embodiments, the high HLB surfactant is present in the composition in an amount of greater than 60 wt. % to about 99.975 wt. %. In some embodiments, the high HLB surfactant is present in the composition in an amount of greater than 60 wt. % to about 90 wt. %. In some embodiments, the high HLB surfactant is present in the composition in an amount of about 70 wt. % to about 85 wt. %.

In some embodiments, the pharmaceutical composition further comprises a low HLB surfactant. As used herein, a “low HLB surfactant” refers to a surfactant with an HLB value of less than 7. Non-limiting examples of low HLB surfactants include, but are not limited to, propylene glycol monocaprylate (e.g., CAPRYOL® 90), propylene glycol dicaprylocaprate (e.g., LABRAFAC® PG), glyceryl monocaprylate (e.g., CAPMUL® 808G), glyceryl monooleate (PECEOL®), and sorbitan monooleate (e.g., SPAN® 80). Accordingly, in some embodiments, the low HLB surfactant is selected from the group consisting of propylene glycol monocaprylate, propylene glycol dicaprylocaprate, glyceryl monocaprylate, glyceryl monooleate, sorbitan monooleate, and a combination of two or more thereof. In some embodiments, the low HLB surfactant is propylene glycol monocaprylate.

In some embodiments, the low HLB surfactant is present in the composition in an amount of about 10 wt. % to about 50 wt. %. This includes about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 wt. %, including any value therebetween. In some embodiments, the low HLB surfactant is present in the composition in an amount of about 10 wt. % to less than 40 wt. %. In some embodiments, the low HLB surfactant is present in the composition in an amount of about 15 wt. % to about 30 wt. %. In some embodiments, wherein the low HLB surfactant is present in the composition in an amount of about 10 wt. % to about 25 wt. %.

In some embodiments, the high HLB surfactant and the low HLB surfactant are present in the composition in a ratio of about 50:50 to about 90:10. In some embodiments, the high HLB surfactant and the low HLB surfactant are present in the composition in a ratio of greater than 60:40 to about 90:10. In some embodiments, the high HLB surfactant and the low HLB surfactant are present in the composition in a ratio of greater than 75:25 to about 90:10. In some embodiments, the high HLB surfactant and the low HLB surfactant are present in the composition in a ratio of greater than 75:25 to about 90:10. In some embodiments, the high HLB surfactant and the low HLB surfactant are present in the composition in a ratio of about 75:25, about 80:20, about 83:17, or about 90:10. In some embodiments, the HLB surfactant and the low HLB surfactant are present in the composition in a ratio of about 75:25.

In some embodiments, the pharmaceutical composition further comprises one or more excipients selected from the group consisting of ethanol, isopropyl alcohol (IPA), benzyl alcohol, propylene glycol, N-methyl-2-pyrrolidone (NMP), glyceryol, dimethylsulfoxide (DMSO), diethylene glycol monoethyl ether (e.g., TRANSCUTOL® HP), poloxamer 124 (e.g., KOLLISOLV® P124), and PEG 400 (e.g., KOLLISOV® PEG 400). In some embodiments, the pharmaceutical composition further comprises one or more excipients selected from the group consisting of ethanol, diethylene glycol monoethyl ether, poloxamer 124, and PEG 400. In some embodiments, the pharmaceutical composition further comprises ethanol. In some embodiments, the pharmaceutical composition further comprises diethylene glycol monoethyl ether. In some embodiments, the pharmaceutical composition further comprises poloxamer 124. In some embodiments, the pharmaceutical composition further comprises PEG 400.

In some embodiments, the one or more excipients are present in the composition in an amount of about 5 wt. % to about 10 wt. %; and the high HLB surfactant is present in the composition in an amount of greater than 54 wt. % to about 89.8 wt. %. This includes about 5, 6, 7, 8, 9, or 10 wt. %, including any value therebetween, of the one or more excipients; and 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89 wt. %, including any value therebetween, of the high HLB surfactant.

In some embodiments, the one or more excipients are present in the composition in an amount of about 5 wt. % to about 10 wt. %; the high HLB surfactant and the low HLB surfactant are present in the composition in a total amount of about 89.8 wt. % to about 94.8 wt. %; and the high HLB surfactant and the low HLB surfactant are present in the composition in a ratio of about 50:50 to about 90:10.

In some embodiments, the one or more excipients are present in the composition in an amount of about 5 wt. % to about 10 wt. %; the high HLB surfactant and the low HLB surfactant are present in the composition in a total amount of about 89.8 wt. % to about 94.8 wt. %; and the high HLB surfactant and the low HLB surfactant are present in the composition in a ratio of greater than 60:40 to about 90:10.

In some embodiments, the one or more surfactants comprise, consist essentially of, or consist of a low HLB surfactant. In some embodiments, the low HLB surfactant is selected from the group consisting of propylene glycol monocaprylate, propylene glycol dicaprylocaprate, glyceryl monocaprylate, glyceryl monooleate, sorbitan monooleate, and a combination of two or more thereof. In some embodiments, the low HLB surfactant is propylene glycol monocaprylate. In some embodiments, the low HLB surfactant is present in the composition in an amount of about 10 wt. % to about 99.975 wt. %. This includes 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 wt. %, including any value therebetween. In some embodiments, the one or more excipients are present in the composition in an amount of about 5 wt. % to about 10 wt. %; and the low HLB surfactant is present in the composition in an amount of about 9 wt. % to about 94.975 wt. %. This includes about 5, 6, 7, 8, 9, or 10 wt. %, including any value therebetween, of the one or more excipients; and about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, or 94 wt. %, including any value therebetween, of the low HLB surfactant.

In some embodiments, the one or more surfactants comprise a medium hydrophilic-lipophilic balance (HLB) surfactant. As used herein, a “medium HLB surfactant” refers to a surfactant with an HLB value of 7 to 10. Non-limiting examples of medium HLB surfactants include, but are not limited to, linoleoyl polyoxyl-6-glycerides (e.g., LABRAFIL® M2125CS), oleoyl polyoxyl-6-glycerides (e.g., LABRAFIL® M1944CS), polyglyceryl-3-oleate and polyglyceryl-10 mono/dioleate (e.g., CAPROL® MPGO), and sorbitan monolaurate (e.g., SPAN™ 20 with HLB of 8.6.

In some embodiments, the pharmaceutical composition comprises a compound of Formula I, or a stereoisomer or a tautomer thereof, or a pharmaceutically acceptable salt thereof, and PEG 40 hydrogenated castor oil.

In some embodiments, the pharmaceutical composition comprises a compound of Formula I, or a stereoisomer or a tautomer thereof, or a pharmaceutically acceptable salt thereof; PEG 40 hydrogenated castor oil; and propylene glycol monocaprylate.

In some embodiments, the pharmaceutical composition comprises a compound of Formula I, or a stereoisomer or a tautomer thereof, or a pharmaceutically acceptable salt thereof, and propylene glycol monocaprylate.

In some embodiments, the pharmaceutical composition consists essentially of a compound of Formula I, or a stereoisomer or a tautomer thereof, or a pharmaceutically acceptable salt thereof, and PEG 40 hydrogenated castor oil.

In some embodiments, the pharmaceutical composition consists essentially of a compound of Formula I, or a stereoisomer or a tautomer thereof, or a pharmaceutically acceptable salt thereof; PEG 40 hydrogenated castor oil; and propylene glycol monocaprylate.

In some embodiments, the pharmaceutical composition consists essentially of a compound of Formula I, or a stereoisomer or a tautomer thereof, or a pharmaceutically acceptable salt thereof, and propylene glycol monocaprylate.

In some embodiments, the pharmaceutical composition consists of a compound of Formula I, or a stereoisomer or a tautomer thereof, or a pharmaceutically acceptable salt thereof, and PEG 40 hydrogenated castor oil.

In some embodiments, the pharmaceutical composition consists of a compound of Formula I, or a stereoisomer or a tautomer thereof, or a pharmaceutically acceptable salt thereof; PEG 40 hydrogenated castor oil; and propylene glycol monocaprylate.

In some embodiments, the pharmaceutical composition consists of a compound of Formula I, or a stereoisomer or a tautomer thereof, or a pharmaceutically acceptable salt thereof, and propylene glycol monocaprylate.

In some embodiments, the pharmaceutical composition consists of:

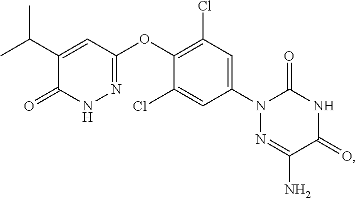 [SMILES: CC(C)c1cc(Oc2c(Cl)cc(-n3nc(N)c(=O)[nH]c3=O)cc2Cl)n[nH]c1=O]

or a pharmaceutically acceptable salt thereof, in an amount of from about 0.1 wt. % to about 0.3 wt. %; and
propylene glycol monocaprylate and PEG 40 hydrogenated castor oil in a ratio of from about 30:70 to about 20:80 (propylene glycol monocaprylate:PEG 40 hydrogenated castor oil).

In some embodiments, the pharmaceutical composition consists of:

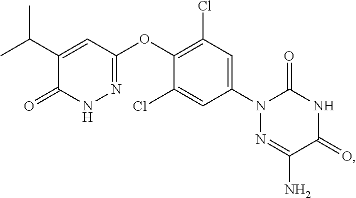 [SMILES: CC(C)c1cc(Oc2c(Cl)cc(-n3nc(N)c(=O)[nH]c3=O)cc2Cl)n[nH]c1=O]

or a pharmaceutically acceptable salt thereof, in an amount of from about 0.1 wt. % to about 0.3 wt. %; and
propylene glycol monocaprylate and PEG 40 hydrogenated castor oil in a ratio of about 30:70 (propylene glycol monocaprylate:PEG 40 hydrogenated castor oil).

In some embodiments, the pharmaceutical composition consists of:

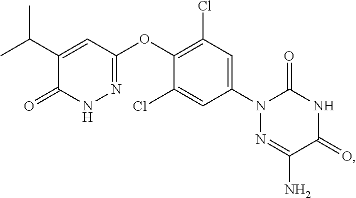 [SMILES: CC(C)c1cc(Oc2c(Cl)cc(-n3nc(N)c(=O)[nH]c3=O)cc2Cl)n[nH]c1=O]

or a pharmaceutically acceptable salt thereof, in an amount of from about 0.1 wt. % to about 0.3 wt. %; and
propylene glycol monocaprylate and PEG 40 hydrogenated castor oil in a ratio of about 25:75 (propylene glycol monocaprylate:PEG 40 hydrogenated castor oil).

In some embodiments, the pharmaceutical composition consists of:

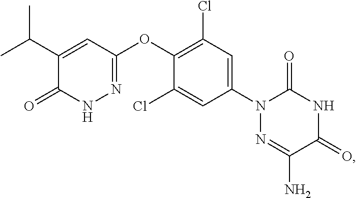 [SMILES: CC(C)c1cc(Oc2c(Cl)cc(-n3nc(N)c(=O)[nH]c3=O)cc2Cl)n[nH]c1=O]

or a pharmaceutically acceptable salt thereof, in an amount of from about 0.1 wt. % to about 0.3 wt. %; and
propylene glycol monocaprylate and PEG 40 hydrogenated castor oil in a ratio of about 20:80 (propylene glycol monocaprylate:PEG 40 hydrogenated castor oil).

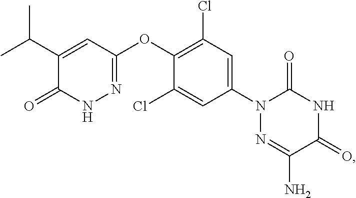 [SMILES: CC(C)c1cc(Oc2c(Cl)cc(-n3nc(N)c(=O)[nH]c3=O)cc2Cl)n[nH]c1=O]

In some embodiments, or the pharmaceutically acceptable salt thereof, is present in an amount of about 0.1, 0.2, 0.3 wt. %, or any value therebetween. In some embodiments,

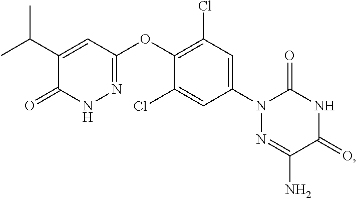 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of about 0.2 wt. %, and propylene glycol monocaprylate and PEG 40 hydrogenated castor oil are present in a ratio of 25:75 (propylene glycol monocaprylate:PEG 40 hydrogenated castor oil).

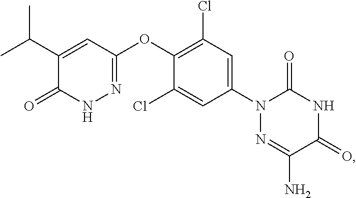 [SMILES: CC(C)c1cc(Oc2c(Cl)cc(-n3nc(N)c(=O)[nH]c3=O)cc2Cl)n[nH]c1=O]

In some embodiments, or the pharmaceutically acceptable salt thereof, is present in an amount of from about 100 μg to about 2000 μg. In some embodiments,

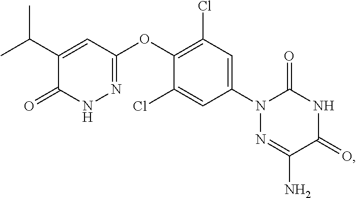 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of about 100, 200, 300, 400, 500, 600, 700, 800, 900, 1000, 1100, 1200, 1300, 1400, 1500, 1600, 1700, 1800, 1900, or 2000 μg, or any value therebetween. In some embodiments,

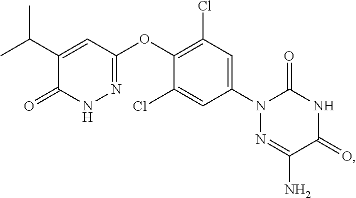 [SMILES: CC(C)c1cc(Oc2c(Cl)cc(-n3nc(N)c(=O)[nH]c3=O)cc2Cl)n[nH]c1=O]

or the pharmaceutically acceptable salt thereof, is present in an amount of about 300, 500, 700, 900, or 1200 μg.

In some embodiments, the composition is in the form of a capsule. In some embodiments, the capsule is a soft gel capsule.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof; PEG 40 hydrogenated castor oil; and propylene glycol monocaprylate; and
the capsule shell comprises, consists essentially of, or consists of gelatin, glycerol, dye, titanium dioxide, and purified water.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.1 wt. % to about 0.3 wt. % of the fill formulation; and propylene glycol monocaprylate and PEG 40 hydrogenated castor oil in a ratio of from about 30:70 to about 20:80 (propylene glycol monocaprylate:PEG 40 hydrogenated castor oil); and
the capsule shell is prepared from an initial composition comprising gelatin, glycerol, dye, titanium dioxide, and purified water; and the initial composition undergoes drying to substantially remove the purified water to prepare the capsule shell.

In some embodiments, the initial composition comprises gelatin in an amount of from about 41 wt. % to about 48 wt. %. This includes about 41, 42, 43, 44, 45, 46, 47, or 48 wt. %, including any value therebetween. In some embodiments, the initial composition comprises gelatin in an amount of 44.13 wt. %.

In some embodiments, the initial composition comprises glycerol in an amount of from about 20 wt. % to about 25 wt. %. This includes about 20, 21, 22, 23, 24, or 25 wt. %, including any value therebetween. In some embodiments, the initial composition comprises glycerol in an amount of 23.13 wt. %.

In some embodiments, the initial composition comprises purified water in an amount of from about 29 wt. % to about 33 wt. %. This includes about 29, 30, 31, 32, or 33 wt. %, including any value therebetween. In some embodiments, the initial composition comprises purified water in an amount of 31.75 wt. %.

In some embodiments, the initial composition comprises titanium dioxide in an amount of from about 0.8 wt. % to about 1.2 wt. %. This includes about 0.8, 0.9, 1.0, 1.1, or 1.2 wt. %. In some embodiments, the initial composition comprises titanium dioxide in an amount of 0.97 wt. %.

In some embodiments, the initial composition comprises dye in an amount of about 0.02 wt. %.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of from about 0.1 wt. % to about 0.3 wt. % of the fill formulation; and propylene glycol monocaprylate and PEG 40 hydrogenated castor oil in a ratio of from about 30:70 to about 20:80 (propylene glycol monocaprylate:PEG 40 hydrogenated castor oil); and
the capsule shell is prepared from an initial composition comprising, consisting essentially of, or consisting of gelatin in an amount of from about 41 wt. % to about 48 wt. % of the initial composition; glycerol in an amount of from about 20 wt. % to about 25 wt. % of the initial composition; dye in an amount of about 0.02 wt. % of the initial composition; titanium dioxide in an amount of about 1 wt. % of the initial composition; and purified water in an amount of from about 29 wt. % to about 33 wt. % of the initial composition; and the initial composition undergoes drying to substantially remove the purified water to prepare the capsule shell.
   As used herein, “substantially remove the purified water” refers to removing at least 50 wt. % of the purified water. This includes about 55 wt. %, 60 wt. %, 65 wt. %, 70 wt. %, 75 wt. %, 80 wt. %, 85 wt. %, 90 wt. %, 95 wt. %, or more, including increments therein, of the purified water. In some embodiments, propylene glycol monocaprylate and PEG 40 hydrogenated castor oil are present in the fill formulation in a ratio of about 30:70, about 25:75, or about 20:80 (propylene glycol monocaprylate:PEG 40 hydrogenated castor oil).

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.1 wt. % of the fill formulation; PEG 40 hydrogenated castor oil in an amount of about 69.95 wt. % of the fill formulation; and propylene glycol monocaprylate in an amount of about 29.95 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.1 wt. % of the fill formulation; PEG 40 hydrogenated castor oil in an amount of about 74.95 wt. % of the fill formulation; and propylene glycol monocaprylate in an amount of about 24.95 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.1 wt. % of the fill formulation; PEG 40 hydrogenated castor oil in an amount of about 79.95 wt. % of the fill formulation; and propylene glycol monocaprylate in an amount of about 19.95 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.2 wt. % of the fill formulation; PEG 40 hydrogenated castor oil in an amount of about 69.90 wt. % of the fill formulation; and propylene glycol monocaprylate in an amount of about 29.90 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.2 wt. % of the fill formulation; PEG 40 hydrogenated castor oil in an amount of about 74.90 wt. % of the fill formulation; and propylene glycol monocaprylate in an amount of about 24.90 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.2 wt. % of the fill formulation; PEG 40 hydrogenated castor oil in an amount of about 79.90 wt. % of the fill formulation; and propylene glycol monocaprylate in an amount of about 19.90 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.2 wt. % of the fill formulation; PEG 40 hydrogenated castor oil in an amount of about 69.85 wt. % of the fill formulation; and propylene glycol monocaprylate in an amount of about 29.95 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.2 wt. % of the fill formulation; PEG 40 hydrogenated castor oil in an amount of about 74.85 wt. % of the fill formulation; and propylene glycol monocaprylate in an amount of about 24.95 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.2 wt. % of the fill formulation; PEG 40 hydrogenated castor oil in an amount of about 79.85 wt. % of the fill formulation; and propylene glycol monocaprylate in an amount of about 19.95 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.3 wt. % of the fill formulation; PEG 40 hydrogenated castor oil in an amount of about 69.8 wt. % of the fill formulation; and propylene glycol monocaprylate in an amount of about 29.9 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.3 wt. % of the fill formulation; PEG 40 hydrogenated castor oil in an amount of about 74.8 wt. % of the fill formulation; and propylene glycol monocaprylate in an amount of about 24.9 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises, consists essentially of, or consists of a compound of Formula I, or a stereoisomer or a tautomer thereof, or a pharmaceutically acceptable salt thereof, in an amount of about 0.3 wt. % of the fill formulation; PEG 40 hydrogenated castor oil in an amount of about 79.8 wt. % of the fill formulation; and propylene glycol monocaprylate in an amount of about 19.9 wt. % of the fill formulation; and
the capsule shell is prepared from an initial composition comprising, consisting essentially of, or consisting of gelatin in an amount of about 44.13 wt. % of the initial composition; glycerol in an amount of about 23.13 wt. % of the initial composition; dye in an amount of about 0.02 wt. % of the initial composition; titanium dioxide in an amount of about 0.97 wt. % of the initial composition; and purified water in an amount of about 31.75 wt. % of the initial composition; and the initial composition undergoes drying to substantially remove the purified water to prepare the capsule shell.

In another aspect, provided herein is a method to prepare the soft gel capsules disclosed herein, the method comprising, consisting essentially of, or consisting of:
(i) preparing a blend formulation consisting of a compound of Formula I, or a stereoisomer or a tautomer thereof, or a pharmaceutically acceptable salt thereof; propylene glycol monocaprylate; and macrogolglycerol hydroxystearate;
(ii) lubricating two gelatin ribbons with a mixture of lecithin and medium-chain triglycerides to provide two lubricated gelatin ribbons;
(iii) continuously and simultaneously feeding the two lubricated gelatin ribbons with the blend formulation between rollers of a rotary die mechanism to form initial capsules; and
(iv) drying and de-lubricating the initial capsules to form the soft gel capsules.
   In some embodiments, step (iii) comprises filling, shaping, sealing, and cutting the initial capsules from the two gelatin ribbons. In some embodiments, the initial capsules are sealed by applying mechanical pressure on die rolls of the die mechanism and heating the initial capsules. In some embodiments, the initial capsules are dried in tumble dryers and de-lubricated with wipers.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and
the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

In some embodiments, the gelatin is present in the initial composition in an amount of from 35 wt. % to about 45 wt. %. This includes 35, 36, 37, 38, 39, 40, 41, 42, 43, 44, or 45 wt. %, including any value therebetween. In some embodiments, the gelatin is present in the initial composition in an amount of about 42 wt. %.

In some embodiments, the mixture of sorbitol and sorbitan is present in the initial composition in an amount of from 8 wt. % to about 15 wt. %. This includes 8, 9, 10, 11, 12, 13, 14, or 15 wt. %, including any value therebetween. In some embodiments, the mixture of sorbitol and sorbitan is present in the initial composition in an amount of about 10 wt. %.

In some embodiments, the glycerin is present in the initial composition in an amount of from 8 wt. % to about 15 wt. %. This includes 8, 9, 10, 11, 12, 13, 14, or 15 wt. %, including any value therebetween. In some embodiments, the glycerin is present in the initial composition in an amount of about 10 wt. %.

In some embodiments, the titanium dioxide is present in the initial composition in an amount of from 1 wt. % to about 2 wt. %. This includes 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, or 2.00 wt. %, including any value therebetween. In some embodiments, the titanium dioxide is present in the initial composition in an amount of about 1.45 wt. %.

In some embodiments, the purified water is present in the initial composition in an amount of from 30 wt. % to about 40 wt. %. This includes 30.00, 30.05, 30.10, 30.15, 30.20, 30.25, 30.30, 30.35, 30.40, 30.45, 30.50, 30.55, 30.60, 30.65, 30.70, 30.75, 30.80, 30.85, 30.90, 30.95, 31.00, 31.05, 31.10, 31.15, 31.20, 31.25, 31.30, 31.35, 31.40, 31.45, 31.50, 31.55, 31.60, 31.65, 31.70, 31.75, 31.80, 31.85, 31.90, 31.95, 32.00, 32.05, 32.10, 32.15, 32.20, 32.25, 32.30, 32.35, 32.40, 32.45, 32.50, 32.55, 32.60, 32.65, 32.70, 32.75, 32.80, 32.85, 32.90, 32.95, 33.00, 33.05, 33.10, 33.15, 33.20, 33.25, 33.30, 33.35, 33.40, 33.45, 33.50, 33.55, 33.60, 33.65, 33.70, 33.75, 33.80, 33.85, 33.90, 33.95, 34.00, 34.05, 34.10, 34.15, 34.20, 34.25, 34.30, 34.35, 34.40, 34.45, 34.50, 34.55, 34.60, 34.65, 34.70, 34.75, 34.80, 34.85, 34.90, 34.95, 35.00, 35.05, 35.10, 35.15, 35.20, 35.25, 35.30, 35.35, 35.40, 35.45, 35.50, 35.55, 35.60, 35.65, 35.70, 35.75, 35.80, 35.85, 35.90, 35.95, 36.00, 36.05, 36.10, 36.15, 36.20, 36.25, 36.30, 36.35, 36.40, 36.45, 36.50, 36.55, 36.60, 36.65, 36.70, 36.75, 36.80, 36.85, 36.90, 36.95, 37.00, 37.05, 37.10, 37.15, 37.20, 37.25, 37.30, 37.35, 37.40, 37.45, 37.50, 37.55, 37.60, 37.65, 37.70, 37.75, 37.80, 37.85, 37.90, 37.95, 38.00, 38.05, 38.10, 38.15, 38.20, 38.25, 38.30, 38.35, 38.40, 38.45, 38.50, 38.55, 38.60, 38.65, 38.70, 38.75, 38.80, 38.85, 38.90, 38.95, 39.00, 39.05, 39.10, 39.15, 39.20, 39.25, 39.30, 39.35, 39.40, 39.45, 39.50, 39.55, 39.60, 39.65, 39.70, 39.75, 39.80, 39.85, 39.90, 39.95, or 40.00 wt. %, including any value therebetween. In some embodiments, the purified water is present in the initial composition in an amount of about 36.55 wt. %.

In some embodiments, the gelatin is present in the initial composition in an amount of from 35 wt. % to about 45 wt. %; the mixture of sorbitol and sorbitan is present in the initial composition in an amount of from 8 wt. % to about 15 wt. %; the glycerin is present in the initial composition in an amount of from 8 wt. % to about 15 wt. %; the titanium dioxide is present in the initial composition in an amount of from 1 wt. % to about 2 wt. %; and the purified water is present in the initial composition in an amount of from 30 wt. % to about 40 wt. %. In some embodiments, the gelatin is present in the initial composition in an amount of about 42 wt. %, the mixture of sorbitol and sorbitan is present in the initial composition in an amount of about 10 wt. %, the glycerin is present in the initial composition in an amount of about 10 wt. %, the titanium dioxide is present in the initial composition in an amount of about 1.45 wt. %, and the purified water is present in the initial composition in an amount of about 36.55 wt. %.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and
the capsule shell is prepared from an initial composition comprising gelatin in an amount of from about 35 wt. % to about 45 wt. % of the initial composition; the mixture of sorbitol and sorbitan in an amount of from about 8 wt. % to about 15 wt. % of the initial composition; glycerin in an amount of from about 8 wt. % to about 15 wt. % of the initial composition; titanium dioxide in an amount of from about 1 wt. % to about 2 wt. % of the initial composition; and purified water in an amount of from about 30 wt. % to about 40 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and
the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and
the capsule shell is prepared from an initial composition comprising gelatin in an amount of from about 35 wt. % to about 45 wt. % of the initial composition; the mixture of sorbitol and sorbitan in an amount of from about 8 wt. % to about 15 wt. % of the initial composition; glycerin in an amount of from about 8 wt. % to about 15 wt. % of the initial composition; titanium dioxide in an amount of from about 1 wt. % to about 2 wt. % of the initial composition; and purified water in an amount of from about 30 wt. % to about 40 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 70:30 to about 80:20 (PEG 40 hydrogenated castor oil:propylene glycol monocaprylate); and
the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 70:30 to about 80:20 (PEG 40 hydrogenated castor oil:propylene glycol monocaprylate); and
the capsule shell is prepared from an initial composition comprising gelatin in an amount of from about 35 wt. % to about 45 wt. % of the initial composition; the mixture of sorbitol and sorbitan in an amount of from about 8 wt. % to about 15 wt. % of the initial composition; glycerin in an amount of from about 8 wt. % to about 15 wt. % of the initial composition; titanium dioxide in an amount of from about 1 wt. % to about 2 wt. % of the initial composition; and purified water in an amount of from about 30 wt. % to about 40 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 70:30 to about 80:20 (PEG 40 hydrogenated castor oil:propylene glycol monocaprylate); and
the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 75:25 (PEG 40 hydrogenated castor oil:propylene glycol monocaprylate); and
the capsule shell is prepared from an initial composition comprising gelatin in an amount of from about 35 wt. % to about 45 wt. % of the initial composition; the mixture of sorbitol and sorbitan in an amount of from about 8 wt. % to about 15 wt. % of the initial composition; glycerin in an amount of from about 8 wt. % to about 15 wt. % of the initial composition; titanium dioxide in an amount of from about 1 wt. % to about 2 wt. % of the initial composition; and purified water in an amount of from about 30 wt. % to about 40 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 75:25 (PEG 40 hydrogenated castor oil:propylene glycol monocaprylate); and
the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 70:30 to about 80:20 (PEG 40 hydrogenated castor oil:propylene glycol monocaprylate); and
the capsule shell is prepared from an initial composition comprising gelatin in an amount of from about 35 wt. % to about 45 wt. % of the initial composition; the mixture of sorbitol and sorbitan in an amount of from about 8 wt. % to about 15 wt. % of the initial composition; glycerin in an amount of from about 8 wt. % to about 15 wt. % of the initial composition; titanium dioxide in an amount of from about 1 wt. % to about 2 wt. % of the initial composition; and purified water in an amount of from about 30 wt. % to about 40 wt. % of the initial composition; and the initial composition undergoes drying to substantially remove the purified water to prepare the capsule shell.

In another aspect, provided herein is a soft gel capsule comprising, consisting essentially of, or consisting of a fill formulation and a capsule shell, wherein:
the fill formulation comprises a compound of Formula I, or a stereoisomer or a tautomer thereof, or a pharmaceutically acceptable salt thereof, in an amount of from about 0.05 wt. % to about 0.4 wt. % of the fill formulation; and PEG 40 hydrogenated castor oil and propylene glycol monocaprylate in a ratio of from about 70:30 to about 80:20 (PEG 40 hydrogenated castor oil:propylene glycol monocaprylate); and
the capsule shell is prepared from an initial composition comprising gelatin in an amount of about 42 wt. % of the initial composition; the mixture of sorbitol and sorbitan in an amount of about 10 wt. % of the initial composition; glycerin in an amount of about 10 wt. % of the initial composition; titanium dioxide in an amount of about 1.45 wt. % of the initial composition; and purified water in an amount of about 36.55 wt. % of the initial composition; and the initial composition undergoes drying to substantially remove the purified water to prepare the capsule shell.

In some embodiments, the gelatin is 160-175 Bloom gelatin. This includes 160, 161, 162, 163, 164, 165, 166, 167, 168, 169, 170, 171, 172, 173, 174, or 175 Bloom gelatin. In some embodiments, the gelatin is 175 Bloom gelatin. In some embodiments, the gelatin is limed bone gelatin. In some embodiments, the gelatin is 175 Bloom limed bone gelatin. In some embodiments, the gelatin is 166 Bloom gelatin. In some embodiments, the gelatin is 166 Bloom STABICAPS™ gelatin.

In another aspect, provided herein is a method to prepare the soft gel capsules disclosed herein, the method comprising, consisting essentially of, or consisting of:
combining (i) a compound of Formula I, or a stereoisomer or a tautomer thereof, or a pharmaceutically acceptable salt thereof; (ii) propylene glycol monocaprylate; and (iii) PEG 40 hydrogenated castor oil under oxygen-free conditions to form a fill formulation;
combining gelatin, a mixture of sorbitol and sorbitan, glycerin, and titanium dioxide in purified water to provide a gelatin mass; and
encapsulating the fill formulation within the gelatin mass followed by drying to form individual soft gel capsules.
   In some embodiments, the compound of Formula I, or the stereoisomer or the tautomer thereof, or the pharmaceutically acceptable salt thereof, is present in the fill formulation in an amount of from about 0.025 wt. % to about 1.0 wt. %. In some embodiments, the compound of Formula I, or the stereoisomer or the tautomer thereof, or the pharmaceutically acceptable salt thereof, is present in the fill formulation in an amount of from about 0.05 wt. % to about 0.4 wt. %. In some embodiments, the PEG 40 hydrogenated castor oil and the propylene glycol monocaprylate are present in the fill formulation in a ratio of from about 50:50 to about 90:10 (PEG 40 hydrogenated castor oil:propylene glycol monocaprylate). In some embodiments, the PEG 40 hydrogenated castor oil and the propylene glycol monocaprylate are present in the fill formulation in a ratio of from about 70:30 to about 80:20 (PEG 40 hydrogenated castor oil:propylene glycol monocaprylate). In some embodiments, the PEG 40 hydrogenated castor oil and the propylene glycol monocaprylate are present in the fill formulation in a ratio of about 75:25 (PEG 40 hydrogenated castor oil:propylene glycol monocaprylate).

In another aspect, provided herein is a soft gel capsule prepared by a method described herein.

### Synthesis of the Compounds

The presently disclosed compounds were synthesized using the general synthetic procedures set forth in US Patent Publication US20200354345A1 (also US granted U.S. Pat. No. 11,091,467).

### Methods of Treatment

In another aspect, disclosed herein are methods of treating a disorder or disease in a subject in need thereof, the method comprising, consisting essentially of, or consisting of administering to the subject a therapeutically effective amount of a pharmaceutical composition disclosed herein, wherein the disorder or disease is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

In another aspect, disclosed herein are methods of treating a thyroid hormone receptor related disorder in a patient, the method comprising, consisting essentially of, or consisting of the steps of identifying a patient in need of treatment for the thyroid hormone receptor related disorder, and administering to the patient, or contacting the patient with, a pharmaceutical composition as described herein.

In some embodiments, a health care professional, such as a physician, physician's assistant, nurse practitioner, or the like, identifies an individual as being in need of treatment for the thyroid hormone receptor related disorder, and/or a candidate for treatment with a pharmaceutical composition disclosed herein. The identification may be based on medical test results, non-responsiveness to other, first-line therapies, the specific nature of the particular liver disorder, or the like.

In some embodiments, the thyroid hormone receptor related disorder is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

In another aspect, disclosed herein are methods of treating MASH in a subject in need thereof, the method comprising, consisting essentially of, or consisting of administering to the subject a therapeutically effective amount of a pharmaceutical composition disclosed herein.

In another aspect, disclosed herein are methods of treating obesity in a subject in need thereof, the method comprising, consisting essentially of, or consisting of administering to the subject a therapeutically effective amount of a pharmaceutical composition disclosed herein.

In another aspect, disclosed herein are methods of treating hyperlipidemia in a subject in need thereof, the method comprising, consisting essentially of, or consisting of administering to the subject a therapeutically effective amount of a pharmaceutical composition disclosed herein.

In another aspect, disclosed herein are methods of treating hypercholesterolemia in a subject in need thereof, the method comprising, consisting essentially of, or consisting of administering to the subject a therapeutically effective amount of a pharmaceutical composition disclosed herein.

In another aspect, disclosed herein are methods of treating diabetes in a subject in need thereof, the method comprising, consisting essentially of, or consisting of administering to the subject a therapeutically effective amount of a pharmaceutical composition disclosed herein.

In another aspect, disclosed herein are methods of treating liver steatosis in a subject in need thereof, the method comprising, consisting essentially of, or consisting of administering to the subject a therapeutically effective amount of a pharmaceutical composition disclosed herein.

In some embodiments, the pharmaceutical composition is administered in combination with a KHK inhibitor, an FXR agonist, a SSAO inhibitor, a FASN inhibitor, or a SCD1 modulator. In some embodiments, the KHK inhibitor is PF-06835919; the FXR agonist is TERN-101 (LY2562175), Tropifexor, obeticholic acid (OCA), or ASC42; the SSAO inhibitor is TERN-201; the FASN inhibitor is ASC40; and the SCD1 modulator is aramchol.

In another aspect, disclosed herein is the use of the pharmaceutical composition as described herein for the manufacture of a medicament for the treatment of a disorder or disease is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

### EXAMPLES

### Example 1. Selection of Excipients Using a Kinetic Solubility Screen

### 1.1—Kinetic Solubility Screen

A kinetic solubility screen covering liquid and semi-solid excipients (listed in Table 1), including those with lipophilic, hydrophilic and amphiphilic structures, was performed in order to evaluate the affinity of compound A with lipid excipients.

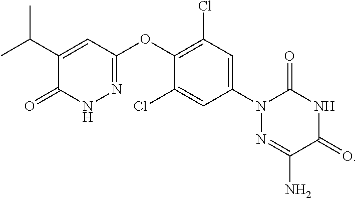 [SMILES: CC(C)c1cc(Oc2c(Cl)cc(-n3nc(N)c(=O)[nH]c3=O)cc2Cl)n[nH]c1=O]

The chemical structure of compound A is

TABLE 1

| Excipients | # | Chemical Name | Trade Name |
|---|---|---|---|
| Lipophilic | 1 | Oleic acid | Oleic acid |
| excipients | 2 | MCT | MCT |
| | 3 | Olive Oil | Olive Oil |
| Low HLB | 4 | Propylene glycol monocaprylate | CAPRYOL ® 90 |
| surfactants | 5 | Propylene glycol | LABRAFAC ® |
| | | dicaprylocaprate | PG |
| | 6 | Glyceryl monocaprylate | CAPMUL ® 808G |
| | 7 | Glyceryl monooleate | PECEOL ® |
| | 8 | Sorbitan monooleate | SPAN ® 80 |
| High HLB | 9 | Polysorbate 80 | TWEEN ® 80 |
| surfactants | 10 | PEG 35 castor oil | KOLLIPHOR ® |
| | | | ELP |
| | 11 | PEG 40 hydrogenated castor oil | KOLLIPHOR ® |
| | | | RH 40 |
| | 12 | PEG 32 lauroyl glycerides | GELUCIRE ® |
| | | | 44/14 |
| | 13 | PEG 32 stearate | GELUCIRE ® |
| | | | 48/16 |
| | 14 | PEG 32 stearoyl glycerides | GELUCIRE ® |
| | | | 50/13 |
| | 15 | D alpha tocopherol PEG 1000 | Vitamin E |
| | | succinate | TPGS |
| Hydrophilic | 16 | Benzyl Benzoate | Benzyl |
| | | | Benzoate |
| excipients | 17 | Ethanol | Ethanol |
| | 18 | Poloxamer 124 | KOLLISOLV ® |
| | | | P124 |
| | 19 | Diethylene glycol monoethyl | TRANSCUTOL ® |
| | | ether | HP |
| | 20 | PEG 400 | KOLLISOLV ® |
| | | | PEG 400 |

Experimental Design: The kinetic solubility screen was performed at 3 concentrations. The initial target concentration was 3 mg/g. In some cases, a lower concentration corresponding to 50% or 25% of the target concentration was tested (i.e., 1.5 mg/g, 0.75 mg/g) by successive dilutions. In some cases, two times and 1.5 times the target concentration (i.e., 6 mg/g and 4.5 mg/g) was evaluated. The solubility screening exercise was performed at 30° C. or 50° C. depending on the melting point of the selected excipients.

Solubilization of compound A was assessed through macroscopic and microscopic examinations. Physical stability study follow-up was performed on samples that sufficiently solubilized compound A during the solubility screening.

Table 2 describes the results of the solubility screen using the 20 excipients, listing the concentrations of solubilized compound A and the experimental conditions.

TABLE 2
Results from solubility screen of excipients

| | | | | Experimental Conditions | |
|---|---|---|---|---|---|
| | Excipients for 17-HPC solubility | | Concentration | Working | |
| | screening exercise | | of solubilized | Temperature | Duration |
| # | Chemical Name | Trade Name | compound | (° C.) | (min) |
| 1 | Oleic acid | Oleic acid | <0.75 mg/g | 30° C. | — |
| 2 | MCT | MCT | <0.75 mg/g | 30° C. | — |
| 3 | Olive Oil | Olive Oil | <0.75 mg/g | 30° C. | — |
| 4 | Propylene glycol | CAPRYOL ® 90 | ~0.75 mg/g | 30° C. | 90 |
| | monocaprylate | | | | |
| 5 | Propylene glycol | LABRAFAC ® | <0.75 mg/g | 30° C. | — |
| | dicaprylocaprate | PG | | | |
| 6 | Glyceryl | CAPMUL ® | >1.5 mg/g | 50° C. | 45 |
| | monocaprylate | 808G | | | |
| 7 | Glyceryl | PECEOL ® | ~0.75 mg/g | 50° C. | 90 |
| | monooleate | | | | |
| 8 | Sorbitan | SPAN ® 80 | ~0.75 mg/g | 50° C. | 90 |
| | monooleate | | | | |
| 9 | Polysorbate 80 | TWEEN ® 80 | >4.5 mg/g | 50° C. | 30 |
| 10 | PEG 35 castor | KOLLIPHOR ® | >1.5 mg/g | 50° C. | 15 |
| | oil | ELP | | | |
| 11 | PEG 40 | KOLLIPHOR ® | >4.5 mg/g | 50° C. | 15 |
| | hydrogenated | RH 40 | | | |
| | castor oil | | | | |
| 12 | PEG 32 lauroyl | GELUCIRE ® | >4.5 mg/g | 50° C. | 90 |
| | glycerides | 44/14 | | | |
| 13 | PEG 32 stearate | GELUCIRE ® | >4.5 mg/g | 50° C. | 15 |
| | | 48/16 | | | |
| 14 | PEG 32 stearoyl | GELUCIRE ® | <0.75 mg/g | 50° C. | — |
| | glycerides | 50/13 | | | |
| 15 | D alpha | Vitamin E TPGS | >1.5 mg/g | 50° C. | 60 |
| | tocopherol PEG | | | | |
| | 1000 succinate | | | | |
| 16 | Benzyl Benzoate | Benzyl Benzoate | <0.75 mg/g | 30° C. | — |
| 17 | Ethanol | Ethanol | >0.75 mg/g | 30° C. | 15 |
| 18 | Poloxamer 124 | KOLLISOLV ® | ~0.75 mg/g | 30° C. | — |
| | | P124 | | | |
| 19 | Diethylene | TRANSCUTOL ® | >6 mg/g | 30° C. | 15 |
| | glycol | HP | | | |
| | monoethyl ether | | | | |
| 20 | PEG 400 | KOLLISOLV ® | >4.5 mg/g | 30° C. | 90 |
| | | PEG 400 | | | |

### 1.2—Physical Stability Screen

The physical stability (Table 3A and 3B) was evaluated for the 14 samples from Table 2 that showed a solubilization of ≥0.75 mg/g. During the evaluation, the liquid formulations were placed under accelerated conditions at 5° C. for one week. All excipients were followed at room temperature for at least 2 weeks and up to 1 month.

TABLE 3A
Physical stability of liquid excipients

| | Excipients for 17-HPC | | Concentration | | Working | Physical Stability | | |
|---|---|---|---|---|---|---|---|---|
| | solubility screening exercise | | of solubilized | | Temperature | 5° C. | RT | RT |
| # | Chemical Name | Trade Name | compound | | (° C.) | (1 week) | (2 weeks) | (1 month) |
| 4 | Propylene glycol | CAPRYOL ® 90 | ~0.75 | mg/g | 30° C. | Stable at | Stable at | Stable at |
| | monocaprylate | | | | | 0.75 mg/g | 0.75 mg/g | 0.75 mg/g |
| 17 | Ethanol | Ethanol | >0.75 | mg/g | 30° C. | Stable at | Stable at | Stable at |
| | | | | | | 0.75 mg/g | 0.75 mg/g | 0.75 mg/g |
| 18 | Poloxamer 124 | KOLLISOLV ® | ~0.75 | mg/g | 30° C. | Stable at | Stable at | Stable at |
| | | P124 | | | | 0.75 mg/g | 0.75 mg/g | 0.75 mg/g |
| 19 | Diethylene glycol | TRANSCUTOL ® | >6 | mg/g | 30° C. | Stable at | Stable at | Stable at |
| | monoethyl ether | HP | | | | 6 mg/g | 6 mg/g | 6 mg/g |
| 20 | PEG 400 | KOLLISOLV ® | >4.5 | mg/g | 30° C. | Stable at | Stable at | Stable at |

| | | | |
|---|---|---|---|
| PEG 400 | 4.5 mg/g | 4.5 mg/g | 4.5 mg/g |

TABLE 3B
Physical stability of semi-solid excipients

| | Excipients for 17-HPC | | Concentration | | Working | Physical Stability | | |
|---|---|---|---|---|---|---|---|---|
| | solubility screening exercise | | of solubilized | | Temperature | RT | | RT |
| # | Chemical Name | Trade Name | compound | | (° C.) | (2 weeks) | | (1 month) |
| 6 | Glyceryl | CAPMUL ® | >1.5 | mg/g | 50° C. | Stable at 1.5 mg/g | | Stable at |
| | monocaprylate | 808G | | | | | | 1.5 mg/g |
| 7 | Glyceryl | PECEOL ® | ~0.75 | mg/g | 50° C. | Stable at 0.75 mg/g | | Stable at |
| | monooleate | | | | | | | 0.75 mg/g |
| 8 | Sorbitan | SPAN ® 80 | ~0.75 | mg/g | 50° C. | Stable at 0.75 mg/g | | Stable at |
| | monooleate | | | | | | | 0.75 mg/g |
| 9 | Polysorbate 80 | TWEEN ® 80 | >4.5 | mg/g | 50° C. | Stable at 4.5 mg/g | | Stable at |
| | | | | | | | | 4.5 mg/g |
| 10 | PEG 35 castor oil | KOLLIPHOR ® | >1.5 | mg/g | 50° C. | Stable at 1.5 mg/g | | Stable at |
| | | ELP | | | | | | 1.5 mg/g |
| 11 | PEG 40 | KOLLIPHOR ® | >4.5 | mg/g | 50° C. | Stable at 4.5 mg/g | | Stable at |
| | hydrogenated | RH 40 | | | | | | 4.5 mg/g |
| | castor oil | | | | | | | |
| 12 | PEG 32 lauroyl | GELUCIRE ® | >4.5 | mg/g | 50° C. | Unstable at | Stable at | Stable at |
| | glycerides | 44/14 | | | | 4.5 mg/g | 3 mg/g | 3 mg/g |
| 13 | PEG 32 stearate | GELUCIRE ® | >4.5 | mg/g | 50° C. | Unstable at | Unstable at | Stable at |
| | | 48/16 | | | | 4.5 mg/g | 3 mg/g | 1.5 mg/g |

| | | | | | | | |
|---|---|---|---|---|---|---|---|
| 15 | D alpha tocopherol | Vitamin E | >1.5 | mg/g | 50° C. | Stable at 1.5 mg/g | Stable at |

| | | |
|---|---|---|
| PEG 1000 succinate | TPGS | 1.5 mg/g |

### 1.3—Chemical Stability Screen

The chemical stability of 14 excipients (Table 4A) was evaluated based on the results from the solubility screen and physical stability screen.

TABLE 4A
List of Excipients for Chemical Stability Screen

| Excipients | # | Chemical Name | Trade Name |
|---|---|---|---|
| Low HLB | 4 | Propylene glycol monocaprylate | CAPRYOL ® 90 |
| surfactants | 6 | Glyceryl monocaprylate | CAPMUL ® 808G |
| | 7 | Glyceryl monooleate | PECEOL ® |
| | 8 | Sorbitan monooleate | SPAN ® 80 |
| High HLB | 9 | Polysorbate 80 | TWEEN ® 80 |
| surfactants | 10 | PEG 35 castor oil | KOLLIPHOR ® ELP |
| | 11 | PEG 40 hydrogenated castor oil | KOLLIPHOR ® RH 40 |
| | 12 | PEG 32 lauroyl glycerides | GELUCIRE ® 44/14 |
| | 13 | PEG 32 stearate | GELUCIRE ® 48/16 |
| | 15 | D alpha tocopherol PEG 1000 succinate | Vitamin E TPGS |
| Hydrophilic | 17 | Ethanol | Ethanol |
| excipients | 18 | Poloxamer 124 | KOLLISOLV ® P124 |
| | 19 | Diethylene glycol monoethyl ether | TRANSCUTOL ® HP |
| | 20 | PEG 400 | KOLLISOLV ® PEG 400 |

Samples were prepared using compound A at concentration of 0.75 mg/g for each selected excipient. The compound was solubilized at 30° C. or 50° C. depending on the melting point of the excipient. The solubilization was evaluated by macroscopic and microscopic evaluation. Samples were placed into glass vials and stored at 40° C. and 75% relative humidity (RH). The chemical stability was monitored through assay and related substances at three time points: 0, 2, and 4 weeks (see Table 4B-4D). The impurity with RRT (relative retention time compared to compound A)=0.94 appeared to be a degradant and was monitored throughout the study as stability-indicating.

TABLE 4B
Appearance of samples after 2 weeks of storage at 40° C. and 75% RH.

| | Excipients | | | |
|---|---|---|---|---|
| # | Chemical Name | Trade Name | Formulation Appearance | Analytical solution appearance |
| 4 | Propylene glycol | CAPRYOL ® 90 | transparent liquid | homogenous transparent solution |
| | monocaprylate | | | |
| 6 | Glyceryl | CAPMUL ® 808G | transparent viscous liquid | homogenous transparent solution |
| | monocaprylate | | | |
| 7 | Glyceryl monooleate | PECEOL ® | transparent viscous liquid | heterogenous solution: 2 phases |
| | | | | are observed |
| 8 | Sorbitan monooleate | SPAN ® 80 | yellow/orange viscous liquid | heterogenous solution: 2 phases |
| | | | | are observed |
| 9 | Polysorbate 80 | TWEEN ® 80 | yellowish viscous liquid | homogenous yellow solution |
| 10 | PEG 35 castor oil | KOLLIPHOR ® ELP | slightly yellowish viscous | homogenous transparent |
| | | | liquid | solution, slightly yellow |
| 11 | PEG 40 hydrogenated | KOLLIPHOR ® RH 40 | transparent viscous liquid | homogenous transparent solution |
| | castor oil | | | |
| 12 | PEG 32 lauroyl | GELUCIRE ® 44/14 | slightly yellowish viscous | opaque solution, slightly yellow |
| | glycerides | | liquid | solution with white deposits |
| 13 | PEG 32 stearate | GELUCIRE ® 48/16 | yellowish viscous liquid | opaque solution, yellowish |
| | | | | solution with deposits |
| 15 | D alpha tocopherol | Vitamin E TPGS | yellowish viscous liquid | homogenous transparent |
| | PEG 1000 succinate | | | solution, yellow solution |
| 17 | Ethanol | Ethanol | transparent liquid | homogenous transparent solution |
| 18 | Poloxamer 124 | KOLLISOLV ® P124 | opaque viscous semi liquid | homogenous transparent solution |
| 19 | Diethylene glycol | TRANSCUTOL ® HP | yellowish viscous liquid | homogenous transparent solution |
| | monoethyl ether | | | |
| 20 | PEG 400 | KOLLISOLV ® PEG 400 | transparent liquid, slight | homogenous transparent solution |
| | | | viscous | |

TABLE 4C
Assay and Impurity Evaluation of Samples after 2 weeks or 4 weeks of storage at 40° C. and 75% RH.

| | Excipients | | Assay (% LC) at 306 nm | | | RRT 0.94 (% area) [at 306 nm] | | |
|---|---|---|---|---|---|---|---|---|
| # | Chemical Name | Trade Name | T0 | T2W | T4W | T0 | T2W | T4W |
| 4 | Propylene glycol | CAPRYOL ® 90 | 100.7 | 101.3 | 99.7 | ND | ND | 0.07 |
| | monocaprylate (type II) | | | | | | | |
| | Glyceryl monocaprylate | CAPMUL ® 808G | 98.3 | 96.8 | 92.9 | 0.27 | 1.64 | 3.42 |
| 7 | Glyceryl monooleate | PECEOL ® | 110.3* | 77.7 | 93.8 | 0.07 | 0.25 | 0.41 |
| 8 | Sorbitan monooleate | SPAN ® 80 | 111.0* | 102.7 | 99.9 | 0.10 | 0.33 | 0.30 |
| 9 | Polysorbate 80 | TWEEN ® 80 | 96.5 | 93.7 | 90.4 | 0.46 | 0.94 | 1.40 |
| | | KOLLIPHOR ® | | | | | | |
| 10 | PEG 35 castor oil | ELP | 98.4 | 96.0 | 95.1 | 0.19 | 0.29 | 0.33 |
| 11 | PEG 40 hydrogenated | KOLLIPHOR ® | 98.7 | 99.3 | 98.8 | 0.07 | 0.09 | 0.13 |
| | castor oil | RH 40 | | | | | | |
| | (macrogolglycerol | | | | | | | |
| | hydroxystearate) | | | | | | | |
| 12 | PEG 32 lauroy1 | GELUCIRE ® | 100.5 | 99.2 | 91 | 0.51 | 0.63 | 3.25 |
| | glycerides | 44/14 | | | | | | |
| 13 | PEG 32 stearate | GELUCIRE ® | 100.0 | 99.2 | 97.6 | 0.51 | 0.76 | 0.89 |
| | | 48/16 | | | | | | |
| 15 | D alpha tocopherol | Vitamin E TPGS | 97.3 | 86.7 | 80.4 | 0.07 | 0.12 | 0.19 |
| | PEG 1000 succinate | | | | | | | |
| 17 | Ethanol | Ethanol | 100.5 | 101.5 | 100.6 | ND | ND | ND |
| 18 | Poloxamer 124 | KOLLISOLV ® | 99.0 | 98.6 | 99.0 | <0.05 | 0.08 | 0.09 |
| | | P124 | | | | | | |
| 19 | Diethylene glycol | TRANSCUTOL ® | 99.4 | 99.4 | 90.9 | ND | 0.13 | 0.69 |
| | monoethyl ether | HP | | | | | | |
| 20 | PEG 400 | KOLLISOLV ® | 97.6 | 94.5 | 67.9 | <0.05 | 0.26 | 0.78 |
| | | PEG 400 | | | | | | |

T0: time at start of experiment; T2W: 2-week mark; T4W: 4-week mark.
*Two phases were observed; compound A was dissolved in less than the total volume, which may explain the high result.

TABLE 4D
Summary of Chemical Stability (Binary Compatibility) after 2 weeks.

| Excipients | # | Chemical Name | Trade Name | Assay | RS |
|---|---|---|---|---|---|
| Low HLB | 4 | Propylene glycol monocaprylate | CAPRYOL ® 90 | acceptable | acceptable |
| surfactants | 6 | Glyceryl monocaprylate | CAPMUL ® 808G | acceptable | not acceptable |
| | 7 | Glyceryl monooleate | PECEOL ® | — | possible |
| | 8 | Sorbitan monooleate | SPAN ® 80 | — | possible |
| High HLB | 9 | Polysorbate 80 | TWEEN ® 80 | possible | not acceptable |
| surfactants | 10 | PEG 35 castor oil | KOLLIPHOR ® ELP | possible | possible |
| | 11 | PEG 40 hydrogenated castor oil | KOLLIPHOR ® RH 40 | acceptable | acceptable |
| | 12 | PEG 32 lauroyl glycerides | GELUCIRE ® 44/14 | acceptable | not acceptable |
| | 13 | PEG 32 stearate | GELUCIRE ® 48/16 | acceptable | not acceptable |
| | 15 | D alpha tocopherol PEG 1000 succinate | Vitamin E TPGS | not acceptable | acceptable |
| Hydrophilic | 17 | Ethanol | Ethanol | acceptable | acceptable |
| excipients | 18 | Poloxamer 124 | KOLLISOLV ® P124 | acceptable | acceptable |
| | 19 | Diethylene glycol monoethyl ether | TRANSCUTOL ® HP | acceptable | acceptable |
| | 20 | PEG 400 | KOLLISOLV ® PEG 400 | not acceptable | not acceptable |

RS = related substances, referring to the impurity at RRT = 0.94

### Example 2. Formulation Development

Formulation compositions having the following ratios of KOLLIPHOR® RH 40/CAPRYOL® 90 were tested: 90/10, 83/17, 75/25, 60/40, and 45/55 (% w/w).

### 2.1—In Vitro Dispersion Testing

In vitro dispersion in water at 37° C. was performed on each formulation to evaluate the capacity to maintain compound A under solubilized state upon dispersion with macroscopic and microscopic follow-up over 24 h. Approximately 1 g of compound A was dispersed into 250 ml of water at 37° C.

TABLE 5
Results of in vitro dispersion testing

| KOLLIPHOR ® RH 40 (%) | | 90 | 83 | 75 | 60 | 45 |
|---|---|---|---|---|---|---|
| CAPRYOL ® 90 (%) | | 10 | 17 | 25 | 40 | 55 |
| API Concentration | | 0.25% | 0.25% | 0.20% | 0.17% | 0.10% |
| Dispersion | Aspect | Clear | Clear | Clear | Translucent | Milky |
| Test | Results | No | No | No | No | No |
| | | precipitation | precipitation | precipitation | precipitation | precipitation |

### 2.2—Rheological Behavior.

The rheological behavior was measured using a HAAKE™ MARS™ rheometer (Thermo Fisher Scientific). Shear ramp: 20 C, [1-1000 s-1], 300 s, #60.

Formulations containing >60 wt. % KOLLIPHOR® RH 40 presented shear thinning behavior and were semi-solid at room temperature. Formulations were also thermo-thinning.

The formulation containing a 90/10 ratio demonstrated a viscosity of greater than ˜2500 m Pa·S, while the other four formulations demonstrated a viscosity of less than ˜2500 m Pa·s

Based on the results discussed above, three formulations were prepared for in vivo evaluation.

### 2.3—In Vivo Formulation Development

Three formulations were tested in an in vivo study by preparing capsules containing the following three formulations A-C:
(A) 83/17 ratio of KOLLIPHOR® RH 40/CAPRYOL® 90 with 0.1% w/w of compound A;
(B) 83/17 ratio of KOLLIPHOR® RH 40/CAPRYOL® 90 with 0.2% w/w of compound A; and
(C) 75/25 ratio of KOLLIPHOR® RH 40/CAPRYOL® 90 with 0.1% w/w of compound A.
   2.4—PK Study: Liquid Formulation Vs. Capsules

The PK of formulations A-C in dog were studied and compared with a liquid formulation (PEG 400:Ethanol (60:40)).

TABLE 6
PK results

| | | | Plasma | Plasma |
|---|---|---|---|---|
| | | | Cₘₐₓ | AUC₀₋₂₄ |
| | Plasma | Plasma | (relative | (relative |
| | Cₘₐₓ | AUC₀₋₂₄ | to liquid | to liquid |
| Formulation | (ng/mL) | (ng h/mL) | formulation) | formulation) |
| Liquid | 405 ± 40.8 | 5517 ± 504 | — | — |
| formulation | | | | |
| Liquid | 405 ± 49.8 | 5547 ± 566 | — | — |
| formulation | | | | |
| (A) | 409 ± 30.4 | 5981 ± 421 | 1.00 | 1.08 |
| (B) | 345 ± 39.4 | 4783 ± 407 | 0.85 | 0.87 |
| (C) | 393 ± 48.3 | 5072 ± 531 | 0.97 | 0.91 |

Overall mean AUC₀₋₂₄/Cₘₐₓ with three capsule formulations were generally similar; however, the exposures with formulation A (83/17, 0.1% compound A) were closest to the liquid formulation.

### 2.5—In Vitro Performance Evaluation—μDISS Testing

The dissolution rate of the formulations A-C was measured in fasted simulated intestinal fluid (FaSSIF), fed simulated intestinal fluid (FeSSIF), and fasted simulated gastric fluid (FaSSGF).

The results showed no variability. Thus, the concentration of compound A had no impact on the dissolution rate in vitro. All three formulations were comparable. All quickly self-emulsified and compound A was quickly solubilized.

### Example 3. Capsule Examples

An oval #5 capsule is used to contain 250 mg of a formulation described herein with 0.2% w/w of compound A.

An oval #10 capsule is used to contain 500 mg of formulation described herein with 0.1% w/w of compound A.

An oval #3 capsule is used to contain 150 mg of formulation described herein with 0.2% w/w of compound A.

### Example 4. Solid (Soft Gel Capsule or “Gelcap”) Formulation

TABLE 7
Composition of solid (gelcap) of compound A (100 μg)

| | Concentration | Weight by Unit |
|---|---|---|
| Ingredient | (% w/w) | (mg/caps) |
| Fill Formulation | | |
| compound A | 0.10 | 0.10 |
| PEG 40 hydrogenated castor oil | 69.95 | 69.95 |
| (macrogolglycerol hydroxystearate) | | |
| Propylene glycol monocaprylate | 29.95 | 29.95 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 5. Solid (Soft Gel Capsule or “Gelcap”) Formulation

TABLE 8
Composition of solid (gelcap) of compound A (100 μg)

| | Concentration | Weight by Unit |
|---|---|---|
| Ingredient | (% w/w) | (mg/caps) |
| Fill Formulation | | |
| compound A | 0.10 | 0.10 |
| PEG 40 hydrogenated castor oil | 74.95 | 74.95 |
| (macrogolglycerol hydroxystearate) | | |
| Propylene glycol monocaprylate | 24.95 | 24.95 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 6. Solid (Soft Gel Capsule or “Gelcap”) Formulation

TABLE 9
Composition of solid (gelcap) of compound A (100 μg)

| | Concentration | Weight by Unit |
|---|---|---|
| Ingredient | (% w/w) | (mg/caps) |
| Fill Formulation | | |
| compound A | 0.10 | 0.10 |
| PEG 40 hydrogenated castor oil | 79.95 | 79.95 |
| (macrogolglycerol hydroxystearate) | | |
| Propylene glycol monocaprylate | 19.95 | 19.95 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 7. Solid (Soft Gel Capsule or “Gelcap”) Formulation

TABLE 10
Composition of solid (gelcap) of compound A (200 μg)

| | Concentration | Weight by Unit |
|---|---|---|
| Ingredient | (% w/w) | (mg/caps) |
| Fill Formulation | | |
| compound A | 0.20 | 0.20 |
| PEG 40 hydrogenated castor oil | 69.90 | 69.90 |
| (macrogolglycerol hydroxystearate) | | |
| Propylene glycol monocaprylate | 29.90 | 29.90 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 8. Solid (Soft Gel Capsule or “Gelcap”) Formulation

TABLE 11
Composition of solid (gelcap) of compound A (200 μg)

| | Concentration | Weight by |
|---|---|---|
| Ingredient | (% w/w) | Unit (mg/caps) |
| Fill Formulation | | |
| compound A | 0.2 | 0.20 |
| PEG 40 hydrogenated castor oil | 74.90 | 74.90 |
| (macrogolglycerol hydroxystearate) | | |
| Propylene glycol monocaprylate | 24.90 | 24.90 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 9. Solid (Soft Gel Capsule or “Gelcap”) Formulation

TABLE 12
Composition of solid (gelcap) of compound A (200 μg)

| | Concentration | Weight by |
|---|---|---|
| Ingredient | (% w/w) | Unit (mg/caps) |
| Fill Formulation | | |
| compound A | 0.2 | 0.20 |
| PEG 40 hydrogenated castor oil | 79.90 | 79.9 |
| (macrogolglycerol hydroxystearate) | | |
| Propylene glycol monocaprylate | 19.90 | 19.90 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 10. Solid (Soft Gel Capsule or “Gelcap”) Formulation

TABLE 13
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.2 | 0.300 |
| PEG 40 hydrogenated castor oil | 69.85 | 104.775 |
| (macrogolglycerol hydroxystearate) | | |
| Propylene glycol monocaprylate | 29.95 | 44.925 |
| (type II) | | |
| Total | 100.00% | 150.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 11. Solid (Soft Gel Capsule or “Gelcap”) Formulation

TABLE 14
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.2 | 0.300 |
| PEG 40 hydrogenated castor oil | 74.85 | 112.275 |
| (macrogolglycerol hydroxystearate) | | |
| Propylene glycol monocaprylate | 24.95 | 37.425 |
| (type II) | | |
| Total | 100.00% | 150.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 12. Solid (Soft Gel Capsule or “Gelcap”) Formulation

TABLE 15
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.2 | 0.300 |
| PEG 40 hydrogenated castor oil | 79.85 | 119.775 |
| (macrogolglycerol hydroxystearate) | | |
| Propylene glycol monocaprylate | 19.95 | 29.925 |
| (type II) | | |
| Total | 100.00% | 150.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 13. Solid (Soft Gel Capsule or “Gelcap”) Formulation

TABLE 16
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.3 | 0.30 |
| PEG 40 hydrogenated castor oil | 69.8 | 69.8 |
| (macrogolglycerol hydroxystearate) | | |
| Propylene glycol monocaprylate | 29.9 | 29.9 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable

### Example 14. Solid (Soft Gel Capsule or “Gelcap”) Formulation

TABLE 17
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.3 | 0.30 |
| PEG 40 hydrogenated castor oil | 74.8 | 74.8 |
| (macrogolglycerol hydroxystearate) | | |
| Propylene glycol monocaprylate | 24.9 | 24.9 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 15. Solid (Soft Gel Capsule or “Gelcap”) Formulation

TABLE 18
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.3 | 0.30 |
| PEG 40 hydrogenated castor oil | 79.8 | 79.8 |
| (macrogolglycerol hydroxystearate) | | |
| Propylene glycol monocaprylate | 19.9 | 19.9 |
| (type II) | | |
| Total | 100.00% | 100.00 |
| Capsule shell (wet) | | |
| Gelatin (bovine skin) | 44.13 | 70.60 |
| Glycerol | 23.13 | 37.01 |
| D&C Yellow 10 | 0.02 | 0.04 |
| Titanium Dioxide | 0.97 | 1.55 |
| Purified water ^{a} | 31.75 | 50.80 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 160.00 |

NA = not applicable
^{a} Purified water is mostly removed during drying steps.
^{b} Will be present only in trace amounts as processing aid and not measurable.

### Example 16. Solid (Soft Gel Capsule or “Gelcap”) Formulation

TABLE 19
Composition of solid (gelcap) of compound A (500 μg)

| | Concentration | Weight by |
|---|---|---|
| Ingredient | (% w/w) | Unit (mg/caps) |
| Fill Formulation | | |
| compound A | 0.20 | 0.50 |
| PEG 40 hydrogenated castor oil | 74.85 | 187.1 |
| (macrogolglycerol hydroxystearate) | | |
| Propylene glycol monocaprylate | 24.95 | 62.4 |
| (type II) | | |
| Total | 100.00% | 250.00 |
| Capsule shell (wet) | | |
| Gelatin (bovine skin) | 44.13 | 97.1 |
| Glycerol | 23.13 | 50.9 |
| D&C Yellow 10 | 0.02 | 0.05 |
| Titanium Dioxide | 0.97 | 2.1 |
| Purified water ^{a} | 31.75 | 69.8 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 220.0 |

NA = not applicable
^{a} Purified water is mostly removed during drying steps.
^{b} Will be present only in trace amounts as processing aid and not measurable.

### Example 17. Solid (Soft Gel Capsule or “Gelcap”) Formulation

TABLE 20
Composition of solid (gelcap) of compound A (700 μg)

| | Concentration | Weight by |
|---|---|---|
| Ingredient | (% w/w) | Unit (mg/caps) |
| Fill Formulation | | |
| compound A | 0.20 | 0.70 |
| PEG 40 hydrogenated castor oil | 74.85 | 262.0 |
| (macrogolglycerol hydroxystearate) | | |
| Propylene glycol monocaprylate | 24.95 | 87.3 |
| (type II) | | |
| Total | 100.00% | 350.00 |
| Capsule shell (wet) | | |
| Gelatin (bovine skin) | 44.13 | 127.5 |
| Glycerol | 23.13 | 66.9 |
| D&C Yellow 10 | 0.02 | 0.07 |
| Titanium Dioxide | 0.97 | 2.8 |
| Purified water ^{a} | 31.75 | 91.7 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 289.0 |

NA = not applicable
^{a} Purified water is mostly removed during drying steps.
^{b} Will be present only in trace amounts as processing aid and not measurable.

### Example 18. Solid (Soft Gel Capsule or “Gelcap”) Formulation

TABLE 21
Composition of solid (gelcap) of compound A (900 μg)

| | Concentration | Weight by |
|---|---|---|
| Ingredient | (% w/w) | Unit (mg/caps) |
| Fill Formulation | | |
| compound A | 0.20 | 0.90 |
| PEG 40 hydrogenated castor oil | 74.85 | 336.8 |
| (macrogolglycerol hydroxystearate) | | |
| Propylene glycol monocaprylate | 24.95 | 112.3 |
| (type II) | | |
| Total | 100.00% | 450.00 |
| Capsule shell (wet) | | |
| Gelatin (bovine skin) | 44.13 | 131.1 |
| Glycerol | 23.13 | 68.7 |
| D&C Yellow 10 | 0.02 | 0.07 |
| Titanium Dioxide | 0.97 | 2.9 |
| Purified water ^{a} | 31.75 | 94.3 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 297.0 |

NA = not applicable
^{a} Purified water is mostly removed during drying steps.
^{b} Will be present only in trace amounts as processing aid and not measurable.

### Example 19. Solid (Soft Gel Capsule or “Gelcap”) Formulation

TABLE 22
Composition of solid (gelcap) of compound A (1200 μg)

| | Concentration | Weight by |
|---|---|---|
| Ingredient | (% w/w) | Unit (mg/caps) |
| Fill Formulation | | |
| compound A | 0.20 | 1.20 |
| PEG 40 hydrogenated castor oil | 74.85 | 449.1 |
| (macrogolglycerol hydroxystearate) | | |
| Propylene glycol monocaprylate | 24.95 | 149.7 |
| (type II) | | |
| Total | 100.00% | 600.00 |
| Capsule shell (wet) | 44.13 | 186.7 |
| Gelatin (bovine skin) | | |
| Glycerol | 23.13 | 97.8 |
| D&C Yellow 10 | 0.02 | 0.1 |
| Titanium Dioxide | 0.97 | 4.1 |
| Purified water ^{a} | 31.75 | 134.3 |
| Soybean lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 423.0 |

NA = not applicable
^{a} Purified water is mostly removed during drying steps.
^{b} Will be present only in trace amounts as processing aid and not measurable.

### Example 20. Soft Gel Capsule Preparation

Soft gel capsules from Examples 4-19 were prepared and packaged using six steps as follows.

### 1. Gelatin Mass Preparation:

Gelatin mass was prepared in a gel melter with the addition of raw materials (i.e., purified water, glycerol and gelatin). After completion of the recipe, the gelatin mass preparation was dispensed in a tank through a sieve. Coloration was performed by adding a mix preparation of dyes and purified water to the gelatin mass preparation.

### 2. Weighing of Blend Formulation:

The macrogolglycerol hydroxystearate was pre-heated and weighed. Compound A and propylene glycol monocaprylate were weighed.

### 3. Blend Formulation Preparation:

Compound A and propylene glycol monocaprylate were transferred into a preparation container. Macrogolglycerol hydroxystearate was poured into the container. The mixture was heated and stirred until the preparation was homogeneous. The active fill material solution was then stored for encapsulation.

### 4. Encapsulation:

The blend formulation was transferred into the hopper. The gelatin ribbons were fed on cooling drums lined up with the die rolls. Two plasticized gelatin ribbons were lubricated with a mix of lecithin and medium-chain triglycerides, and continuously and simultaneously fed with the blend formulation between the rollers of the rotary die mechanism where the capsule was simultaneously filled, shaped, sealed and cut from the gelatin ribbon. The sealing of the capsule was achieved by mechanical pressure on the die rolls and the heating of the ribbons by the wedge. Formed capsules were transferred into tumble dryers where they were pre-dried and de-lubricated with wipers.

### 5. Drying:

After the pre-drying step, the capsules were collected on trays. Trays were stored in drying corridors where dry air was blown in until completion of drying which was controlled through a capsule hardness test.

### 6. Bulk Packaging:

Capsules were bulk packaged into double polyethylene bags.

### Example 21. Open-Label, Single-Dose, Three-Period, Fixed-Sequence Crossover Study

The relative bioavailability of the solid (gelcap) formulation of compound A of the present disclosure versus solution formulation of compound A, and food effect of the gelcap formulation were assessed. One cohort of 8 healthy human subjects received the following single doses of compound A in a fixed sequence: 1) as a solution in a fasted state, 2) as a solid formulation in a fasted state, and 3) as a solid formulation in a fed state, separated by a washout period of at least 10 days. Subjects were followed-up for approximately 2 weeks after the administration of the last dose of study drug.

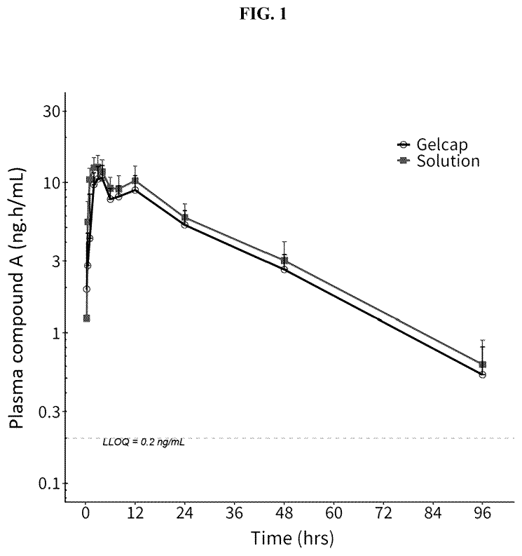

The mean plasma concentration-time plots for compound A following single oral doses of 0.6 mg compound A of solution (diluent=60% v/v polyethylene glycol 400 and 40% v/v ethanol) and solid (two gelcaps of Example 11, each with 300 μg of compound A) formulations under fasted conditions are shown in FIG. 1. Pharmacokinetic (PK) parameters are shown in Table 23.

A statistical comparison of PK parameters for solid (gelcap) versus solution formulations was performed. Geometric mean ratios for Cₘₐₓ, AUCₜ and AUC_{inf} were 86.5%, 85% and 85%, respectively, for gelcap versus solution formulations.

TABLE 23
Plasma PK Parameters [Geometric Means (Geometric SD)] of
Compound A Following Single Oral Compound A Doses of Solution
and Solid Formulations under Fasted Condition in Healthy Subjects

| | No. of | AUC₀₋₂₄ | AUC_{0-inf} | Cₘₐₓ | tₘₐₓ ^{a} | t_{1/2} ^{b} |
|---|---|---|---|---|---|---|
| | Subjects | (ng · h/mL) | (ng · h/mL) | (ng/mL) | (h) | (h) |
| Gelcap/Fasted | 8 | 178 (19.2) | 347(22.8) | 11.1 (18) | 3 (2, 4) | 23.6(5.01) |
| Solution/Fasted | 8 | 210 (21.7) | 403(23.7) | 12.8 (16.6) | 3 (2, 4) | 24.1(5.21) |

AUC₀₋₂₄ = area under the concentration-time curve from 0 to 24 hours; AUC_{0-inf} = area under the concentration-time curve from 0 to infinity; Cₘₐₓ = maximum concentration; tₘₐₓ = time to reach maximum concentration; tₘₐₓ = plasma terminal half-life.
^{a} Median (minimum, maximum).
^{b} Arithmetic mean (standard deviation).

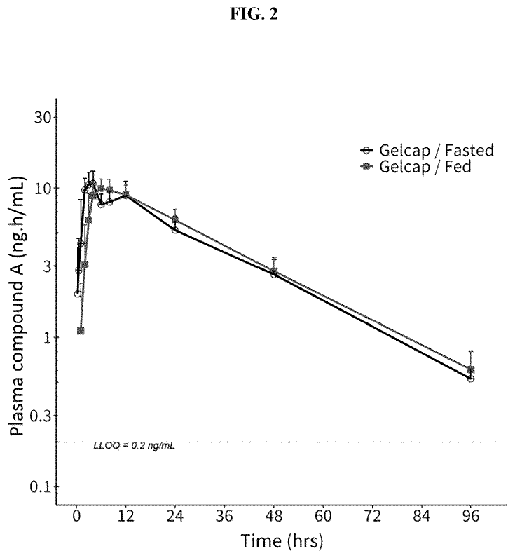

The mean plasma concentration-time plots for compound A following single oral doses of 0.6 mg compound A of solid formulations under fasted vs fed conditions is shown in FIG. 2. Pharmacokinetic (PK) parameters are summarized in Table 24.

A statistical comparison of PK parameters for fed versus fasted for the solid (gelcap) formulation was performed. Geometric mean ratios for Cₘₐₓ, AUCₜ and AUC_{inf} were 95.4%, 101% and 101% for fed versus fasting conditions, respectively. The associated 90% confidence intervals for all 3 parameters were contained within 80% and 125%, meeting the bioequivalence criteria with respect fed versus fasted which demonstrate that there is no food effect for the gelcap formulation.

TABLE 24
Plasma PK Parameters [Geometric Means (Geometric SD)] of
Compound A Following Single Oral Compound A Doses of Solid Formulations
under Fasted and/or Fed Conditions in Healthy Subjects

| Solid Dosage | No. of | AUC₀₋₂₄ | AUC_{0-inf} | Cₘₐₓ | tₘₐₓ ^{a} | t_{1/2} ^{b} |
|---|---|---|---|---|---|---|
| Form | Subjects | (ng · h/mL) | (ng · h/mL) | (ng/mL) | (h) | (h) |
| Gelcap/Fasted | 8 | 178(19.2) | 347(22.8) | 11.1(18) | 3(2, 4) | 23.6(5.01) |
| Gelcap/Fed | 8 | 180(13.7) | 369(17.2) | 10.5(14.7) | 7(4, 12) | 22.9(3.61) |

AUC₀₋₂₄ = area under the concentration-time curve from 0 to 24 hours; AUC_{0-inf} = area under the concentration-time curve from 0 to infinity; Cₘₐₓ = maximum concentration; tₘₐₓ = time to reach maximum concentration; tₘₐₓ = plasma terminal half-life.
^{a} Median (minimum, maximum)
^{b} Arithmetic mean (standard deviation)

### Example 22. Assessment of Shelf Stability on In Vitro Dissolution

The soft gel capsule from Example 18 was assessed for impact of shelf stability on dissolution. Test samples were stored for time=3, 6, 9, or 12 months at 25° C. and 60% relative humidity (RH) and for time=1, 3, or 6 months at 40° C. and 75% RH. Control samples (time=0 months) were tested for their dissolution alongside the test samples. Dissolution conditions were conducted according to USP <711> and Ph. Eur. 2.9.3. In particular, dissolution was determined using USP <711> Apparatus II, with a dissolution media of 0.1 N. hydrochloric acid in 900 mL and a paddle speed of 75 rpm at 37° C.±0.5° C. Enzyme (pepsin) was added to the dissolution media when cross-linking was observed and documented. Enzyme addition mimics in vivo conditions, since enzymes in the digestive system would usually assist in breakdown of a capsule shell which has undergone crosslinking. Analysis was performed with an HPLC method, using an X-Bridge C18, 150×4.6 mm, 3.5 μm column (or equivalent) equilibrated at 30° C., an isocratic elution with 0.05% TFA in water/acetonitrile (50/50, v/v) as the mobile phase and a UV detection wavelength of 306 nm.

Test samples stored for time=3, 6, or 12 months at 25° C. and 60% RH were dissolved without enzyme. Control sample (“Initial”; storage time of t=0 months) was tested alongside these test samples. Their dissolution is shown in FIG. 3. The data indicates minimal effect on the dissolution profile due to storage for up to 12 months at 25° C. and 60% relative humidity.

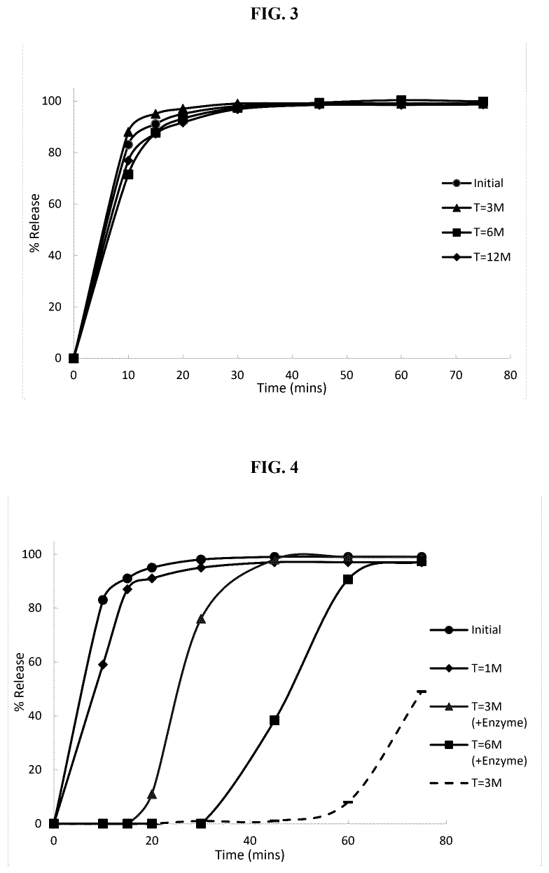

However, test samples which were stored for 1, 3, or 6 months at 40° C. and 75% RH were dissolved with or without enzyme and demonstrated increasingly poor dissolution after 3-month storage (select data shown in FIG. 4). Assessment of the test sample stored for 3 months in dissolution media with enzyme exhibited better dissolution compared to the same sample tested in enzyme-free dissolution media, further indicating that crosslinking of the capsule shell slowed down in vitro dissolution in the absence of enzyme. Control sample (“Initial”; storage time of t=0 months) was tested alongside these test samples.

### Example 23. Solid (Soft Gel Capsule or “Gelcap”) Formulation

TABLE 24
Composition of solid (gelcap) of compound A (300 μg)

| | Concentration | Weight by |
|---|---|---|
| Ingredient | (% w/w) | Unit (mg/caps) |
| Fill Formulation | | |
| compound A | 0.07 | 0.30 |
| PEG 40 hydrogenated castor oil | 74.96 | 337.3 |
| (macrogolglycerol hydroxystearate) | | |
| Propylene glycol monocaprylate | 24.98 | 112.4 |
| (type II) | | |
| Total | 100.00% | 450.00 |
| Capsule shell (wet) | | |
| DMF 014194* | 98.55% | 795.3 |
| Titanium Dioxide | 1.45% | 11.7 |
| Lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 807.0 |

*DMF 014194: composition consisting of 35 wt. %-45 wt. % gelatin (175 Bloom limed bone or 166 Bloom STABICAPS ™), 8 wt. %-15 wt. % glycerin, 8 wt. %-15 wt. % sorbitol special (a mixture of sorbitol and sorbitan), and 30 wt. %-40 wt. % purified water, wt. % relative to initial composition also containing titanium dioxide prior to drying to substantially remove the water; commercially available from Pantheon.
NA = not applicable
^{a} Purified water is mostly removed during drying steps.
^{b} Will be present only in trace amounts as processing aid and not measurable.

### Example 24. Solid (Soft Gel Capsule or “Gelcap”) Formulation

TABLE 25
Composition of solid (gelcap) of compound A (700 μg)

| | Concentration | Weight by |
|---|---|---|
| Ingredient | (% w/w) | Unit (mg/caps) |
| Fill Formulation | | |
| compound A | 0.16 | 0.70 |
| PEG 40 hydrogenated castor oil | 74.89 | 337.0 |
| (macrogolglycerol hydroxystearate) | | |
| Propylene glycol monocaprylate | 24.95 | 112.3 |
| (type II) | | |
| Total | 100.00% | 450.00 |
| Capsule shell (wet) | | |
| DMF 014194* | 98.55% | 795.3 |
| Titanium Dioxide | 1.45% | 11.7 |
| Lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 807.0 |

*DMF 014194: composition consisting of 35 wt. %-45 wt. % gelatin (175 Bloom limed bone or 166 Bloom STABICAPS ™), 8 wt. %-15 wt. % glycerin, 8 wt. %-15 wt. % sorbitol special (a mixture of sorbitol and sorbitan), and 30 wt. %-40 wt. % purified water, wt. % relative to initial composition also containing titanium dioxide prior to drying to substantially remove the water; commercially available from Pantheon.
NA = not applicable
^{a} Purified water is mostly removed during drying steps.
^{b} Will be present only in trace amounts as processing aid and not measurable.

### Example 25. Solid (Soft Gel Capsule or “Gelcap”) Formulation

TABLE 26
Composition of solid (gelcap) of compound A (900 μg)

| | Concentration | Weight by |
|---|---|---|
| Ingredient | (% w/w) | Unit (mg/caps) |
| Fill Formulation | | |
| compound A | 0.20 | 0.90 |
| PEG 40 hydrogenated castor oil | 74.84 | 336.8 |
| (macrogolglycerol hydroxystearate) | | |
| Propylene glycol monocaprylate | 24.96 | 112.3 |
| (type II) | | |
| Total | 100.00% | 450.00 |
| Capsule shell (wet) | | |
| DMF 014194* | 98.55% | 795.3 |
| Titanium Dioxide | 1.45% | 11.7 |
| Lecithin | NA | Trace ^{b} |
| Medium chain triglycerides for | NA | Trace ^{b} |
| internal ribbon lubrication | | |
| Medium chain triglycerides for | NA | Trace ^{b} |
| external ribbon lubrication | | |
| Total | 100.00% | 807.0 |

*DMF 014194: composition consisting of 35 wt. %-45 wt. % gelatin (175 Bloom limed bone or 166 Bloom STABICAPS ™), 8 wt. %-15 wt. % glycerin, 8 wt. %-15 wt. % sorbitol special (a mixture of sorbitol and sorbitan), and 30 wt. %-40 wt. % purified water, wt. % relative to initial composition also containing titanium dioxide prior to drying to substantially remove the water; commercially available from Pantheon.
NA = not applicable
^{a} Purified water is mostly removed during drying steps.
^{b} Will be present only in trace amounts as processing aid and not measurable.

### Example 26. Soft Gel Capsule Preparation

Soft gel capsules from Examples 23-25 were prepared and packaged using the following steps. Nitrogen overlaying was performed throughout the dispensing, compounding, and encapsulation processes.

### 1. Gelatin Mass Compounding:

Gelatin mass was prepared with the addition of the raw materials (purified water, gelatin, glycerin, and sorbitol-sorbitan mixture) and deaerated. Following the gelatin mass preparation, titanium dioxide (TiO₂) dispersed in glycerin was added to the Gelatin to complete the manufacture. After preparation, the gelatin mass was held under heat (135-145° F.) until encapsulation.

### 2. Dispensing of Components for Fill Solution:

Previously unopened containers of macrogolglycerol hydroxystearate and propylene glycol monocaprylate were used to prepare the fill solution.
Compound A and propylene glycol monocaprylate were weighed in appropriate container as needed.
Macrogolglycerol hydroxystearate was melted in an oven, stirred (100±50 rpm), and dispensed into a jacketed bulk mixing tank. The tank was maintained at 40±5° C. and was under nitrogen overlay.

### 3. Compounding of the Fill Solution:

Propylene glycol monocaprylate (portion I) was added to the mixing tank containing macrogolglycerol hydroxystearate. The resultant mixture was heated (40±5° C.) and stirred for at least 20 minutes until homogeneous. In a separate container, Compound A was added to propylene glycol monocaprylate (portion II), and the resultant mixture was stirred to form a slurry. The Compound A-containing slurry was then slowly added with stirring (300±150 rpm) to the mixing tank containing propylene glycol monocaprylate (portion I) and macrogolglycerol hydroxystearate. The slurry container was rinsed with propylene glycol monocaprylate (portion III) multiple times, and the rinsate added to the mixing tank. The resultant mixture was heated (40±5° C.) and stirred (300±150 rpm) for at least 1 hour until Compound A was completely dissolved and a homogeneous solution was obtained, providing the fill solution. During this stirring process, the mixture was sampled and evaluated for blend uniformity and visually checked for the absence of undissolved particles under a microscope.
The fill solution was de-aerated under vacuum with stirring (20±5 rpm) and purged with nitrogen multiple times before being stored for encapsulation under a nitrogen blanket, with constant heating (32±3° C.) and stirring.

### 4. Encapsulation:

The fill solution and gelatin mass were connected to an automated soft gel encapsulator using heated hoses. During encapsulation, the fill solution was continuously mixed and heated. The gelatin ribbons were fed on cooling drums lined up with die rolls. Two gelatin ribbons were simultaneously fed with the fill solution between the rollers of the rotary die where the capsule was shaped, sealed, and cut from the gelatin ribbon. Sealing was achieved by mechanical pressure on the die rolls and heating of the ribbons by a wedge. Formed capsules were pre-dried by tumbling in drying baskets attached to the encapsulation machine.
During encapsulation, the wedge temperature, gelatin mass and fill solution temperature were monitored. Capsules were evaluated for ribbon thickness, fill weight, and trailing seam thickness throughout the run.

### 5. Drying:

After the pre-drying process, capsules were collected on trays and dried in drying tunnels. Capsules were pulled at specified intervals and tested for hardness. Drying was completed when the target hardness was achieved. Capsules were then run through a shaker box and transferred to deep trays to be visually inspected, sized, checked for leakers and metal particles.

### 6. Packaging:

After passing through metal detector, the soft gel capsules were bulk packaged in lock-tied double polyethylene bags in cardboard box. Soft gel capsules were eventually packaged n 60 cc HDPE bottles with CRC polypropylene caps.

### Example 27. Assessment of Shelf Stability on In Vitro Dissolution

The soft gel capsules from Example 25 were assessed for impact of shelf stability on dissolution. Test samples were stored for time=1, 3, or 6 months at 40° C. and 75% RH. A control sample (time=0 months) (“Initial”) was tested for its dissolution alongside the test samples. Dissolution conditions were as described in Example 22. No enzyme was added to the dissolution media for this study.

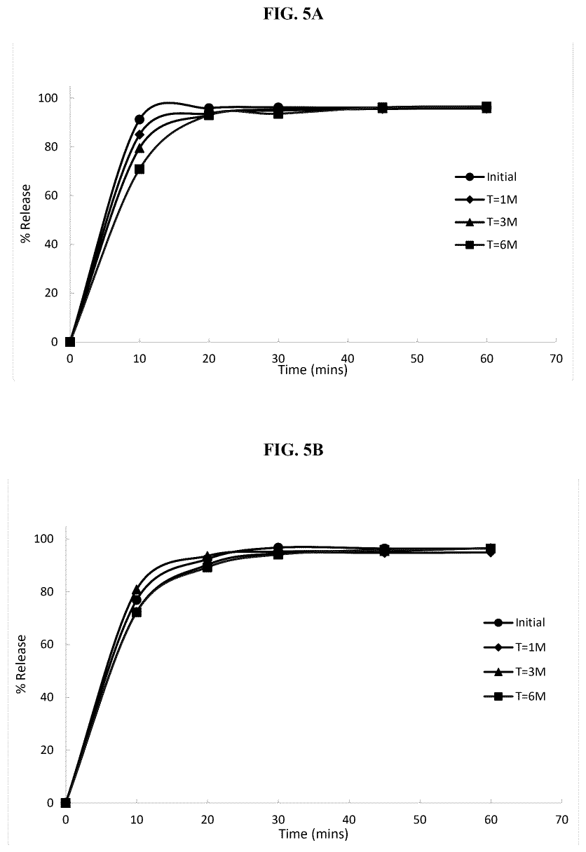

As shown in FIG. 5A and FIG. 5B, test samples with 175 Bloom limed bone gelatin or 166 Bloom gelatin (STABICAPS™), respectively, exhibited minimal effect on the respective dissolution profile due to storage for up to 6 months at 40° C. and 75% RH. The combination of fill preparation under inert atmosphere (e.g., nitrogen) and use of freshly opened containers of reagents as described in Example 26 and the change in composition of the capsule shell in Example 25 were necessary to mitigate the crosslinking effect observed in Example 19.

### Example 28. Relative Bioavailability Between Soft Gel Capsule Formulations

The pharmacokinetics of soft gel capsules of Examples 17 and 24 were compared in 16 healthy human subjects in a single dose, open label, randomized, two-period, two-sequence crossover Phase I study. One cohort of 8 subjects received a single oral dose of a soft gel capsule (0.7 mg compound A) of Example 17 followed by a washout period of at least 7 days before administration of a single oral dose of a soft gel capsule (0.7 mg compound A) of Example 24. The other cohort of 8 subjects received a single oral dose of a soft gel capsule (0.7 mg compound A) of Example 24 followed by a washout period of at least 7 days before administration of a single oral dose of a soft gel capsule (0.7 mg compound A) of Example 17. Subjects were followed-up for 2 weeks after the administration of the last dose of soft gel capsule formulation.

Data from this study is shown in Table 27. This study demonstrates that the two formulations are bioequivalent.

TABLE 27
Relative PK data

| | | ^{†}AUCₗₐₛₜ | ^{†}AUC_{inf} | ^{†}Cₘₐₓ | *Tₘₐₓ | ^{#}T_{1/2} |
|---|---|---|---|---|---|---|
| Formulation | n | (ng*h/mL) | (ng*h/mL) | (ng/mL) | (h) | (h) |
| Example 17 | 8 | 528 (11.3) | 551 (10.6) | 21.7 (16.3) | 3 (2, 4) | 20.8 (2.94) |
| Example 24 | 8 | 548 (13.5) | 571 (13) | 22.8 (13.1) | 2 (1, 3) | 20 (2.62) |
| Geometric mean ratio | | 1.04 | 1.04 | 1.05 | | |
| (Example 24:Example 17) | | | | | | |
| 90% Cl (lower-upper) | | 0.96-1.12 | 0.96-1.12 | 0.98-1.13 | | |

^{†}Data shown as geometric mean (coefficient of variation) for AUCₗₐₛₜ, AUC_{inf}, Cₘₐₓ;
*Data shown as median (minimum, maximum) for Tₘₐₓ;
^{#}Data shown as mean (standard deviation) for T_{1/2}

### REFERENCES

1. Younossi, Z M, Koenig, A B, Abdelatif, D, Fazel, Y, Henry, L, Wymer, M. Global epidemiology of nonalcoholic fatty liver disease-Meta-analytic assessment of prevalence, incidence, and outcomes. Hepatology, 2016, 64(1):73e84.
2. Gastroenterology. 2012 June; 142(7):1592-609. doi: 10.1053/j.gastro.2012.04.001. Epub 2012 May 15.
3. Serfaty, L., Lemoine, M. Definition and natural history of metabolic steatosis: clinical aspects of NAFLD, NASH and cirrhosis. Diabetes and Metabolism, 2008, 34 (6 Pt 2):634e637.
4. Hepatology. 2012 October; 56(4):1580-1584. doi: 10.1002/hep.26031
5. Dulai, P S, Singh, S, Patel, J, Soni, M, Prokop, L J, Younossi, Z, et al., Increased risk of mortality by fibrosis stage in nonalcoholic fatty liver disease: systematic review and meta-analysis. Hepatology, 2017, 65(5):1557e1565.
6. Younossi, Z M, Loomba, R, Rinella, M E, Bugianesi, E, Marchesini, G, Neuschwander-Tetri, B A, et al. Current and future therapeutic regimens for non-alcoholic fatty liver disease (NAFLD) and non-alcoholic steatohepatitis (NASH). Hepatology, 2018, 68(1):349e360.
7. Harvey C B, Williams G R. Mechanism of thyroid hormone action. Thyroid, 2002 June; 12(6):441-6.
8. Bookout A L, Jeong Y, Downes M, Yu R T, Evans R M, Mangelsdorf D J. Anatomical profiling of nuclear receptor expression reveals a hierarchical transcriptional network. Cell, 2006, 126:789-799
9. Flamant F, Baxter J D, Forrest D, Refetoff S, Samuels H H, Scanlan T S, Vennstrom B, Samarut J. International union of pharmacology. LIX. The pharmacology and classification of the nuclear receptor superfamily: thyroid hormone receptors. Pharmacol. Rev., 2006, 58:705-711
10. Haning H, Woltering M, Mueller U, Schmidt G, Schmeck C, Voehringer V, Kretschmer A, Pernerstorfer J. Bioorg. Med Chem Lett., 2005 Apr. 1, 15(7):1835-40. Novel heterocyclic thyromimetics.
11. Hirano T, Kagechika H. Thyromimetics: a review of recent reports and patents (2004-2009). Expert Opin Ther Pat., 2010 February; 20(2):213-28. doi: 10.1517/13543770903567069.
12. Kowalik M A, Columbano A, Perra A. Thyroid Hormones, Thyromimetics and Their Metabolites in the Treatment of Liver Disease. Front Endocrinol (Lausanne), 2018 Jul. 10; 9:382. doi: 10.3389/fendo.2018.00382. eCollection 2018.
13. Erion M D, Cable E E, Ito B R, Jiang H, Fujitaki J M, Finn P D, Zhang B H, Hou J, Boyer S H, van Poelje P D, Linemeyer D L. Targeting thyroid hormone receptor-beta agonists to the liver reduces cholesterol and triglycerides and improves the therapeutic index. Proc Natl Acad Sci USA., 2007 Sep. 25; 104(39):15490-5. Epub 2007 Sep. 18.
14. Hartley M D, Kirkemo L L, Banerji T, Scanlan T S. A Thyroid Hormone-Based Strategy for Correcting the Biochemical Abnormality in X-Linked Adrenoleukodystrophy. Endocrinology 2017, 158(5), p 1328-1338. doi: 10.1210/en.2016-1842.
15. Milanesi A, Brent G A. Beam Me In: Thyroid Hormone Analog Targets Alternative Transporter in Mouse Model of X-Linked Adrenoleukodystrophy. Endocrinology 2017, 158, p 1116-1119. doi: 10.1210/en.2017-00206.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. A pharmaceutical composition comprising a compound of Formula I: 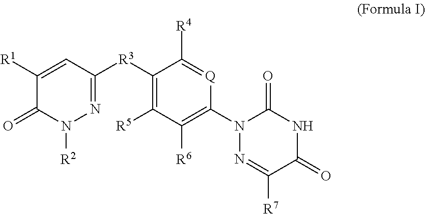
or a stereoisomer or a tautomer thereof, or a pharmaceutically acceptable salt thereof, and one or more surfactants;
wherein:
R¹ is C₁-C₆ alkyl; C₂-C₁₀ alkenyl; a non-aromatic C₃-C₁₂ carbocyclic ring; a C₆-C₁₀ aryl group; a 3- to 6-membered heterocycloalkyl ring containing one to four ring heteroatoms independently selected from oxygen, sulfur, and nitrogen; a five- to ten-membered heteroaryl ring containing one to four ring heteroatoms independently selected from oxygen, sulfur, and nitrogen; a (carbocyclic)alkyl group; an aralkyl group; or a (heterocycloalkyl)alkyl group; and R¹ is optionally substituted with one to five R^{g} independently selected from the group consisting of hydroxy, halogen, CN, C₁-C₆ alkyl, C₆-C₁₀ aryl, C₁-C₆ alkoxy, C₁-C₆ haloalkoxy, and C₆-C₁₀ aralkoxy, or two R^{g} together with the atoms to which they are attached form an aromatic or non-aromatic 3- to 6-membered ring, optionally containing one or two ring heteroatoms independently selected from oxygen, sulfur, and nitrogen;
R² is hydrogen or an optionally substituted C₁-C₆ alkyl;
R³ is independently —(C(R^{a})₂)_{z}—; oxygen; sulfur; or —NR^{a}—; wherein:
each R^{a} is independently a hydrogen or C₁-C₆ alkyl; and
z is 0, 1, 2, 3, 4 or 5;
each of R⁴ and R⁵ is independently selected from halogen, —CN, optionally substituted C₁-C₆ alkyl, optionally substituted C₁-C₆ alkoxy, optionally substituted C₂-C₆ alkenyl, or cyclopropyl;
R⁶ is selected from hydrogen, optionally substituted C₁-C₆ alkyl, optionally substituted C₁-C₆ alkoxy, cyano, or halogen;
optionally R⁵ and R⁶ taken together, along with the carbon atoms to which they are attached, form a 4-, 5- or 6-membered non-aromatic carbocyclic, heterocycloalkyl, aryl, or heteroaryl ring;
Q is nitrogen or —CR^{c}—, wherein R^{c} is hydrogen, halogen, or C₁-C₆ alkyl;
R⁷ is —(C(R⁴)₂)ₙ—N(R^{d})₂; wherein
each R^{d} is independently hydrogen or optionally substituted C₁-C₆ alkyl; and
each n is independently selected from 0 or 1; and
wherein the composition is formulated for oral administration.

2. The pharmaceutical composition of claim 1, wherein the compound is present in the composition in an amount of about 0.025 wt. % to about 1.0 wt. %.

3. The pharmaceutical composition of claim 1, wherein the one or more surfactants comprise a high HLB surfactant.

4. The pharmaceutical composition of claim 3, wherein the high HLB surfactant is selected from the group consisting of polysorbate 80, PEG 35 castor oil, PEG 40 hydrogenated castor oil, PEG 32 lauroyl glycerides, PEG 32 stearate, PEG 32 stearoyl glycerides, D alpha tocopherol PEG 1000 succinate, and any combination of two or more thereof.

5. (canceled)

6. (canceled)

7. The pharmaceutical composition of claim 3, further comprising a low HLB surfactant.

8. The pharmaceutical composition of claim 7, wherein the low HLB surfactant is selected from the group consisting of propylene glycol monocaprylate, propylene glycol dicaprylocaprate, glyceryl monocaprylate, glyceryl monooleate, sorbitan monooleate, and a combination of two or more thereof.

9.-17. (canceled)

18. The pharmaceutical composition of claim 7, wherein the high HLB surfactant and the low HLB surfactant are present in the composition in a ratio of about 50:50 to about 90:10.

19.-21. (canceled)

22. The pharmaceutical composition of claim 7, further comprising one or more excipients selected from the group consisting of ethanol, isopropyl alcohol (IPA), benzyl alcohol, propylene glycol, N-methyl-2-pyrrolidone (NMP), glyceryol, dimethylsulfoxide (DMSO), diethylene glycol monoethyl ether, poloxamer 124, and PEG 400.

23. The pharmaceutical composition of claim 22, wherein the one or more excipients are present in the composition in an amount of about 5 wt. % to about 10 wt. %; the high HLB surfactant and the low HLB surfactant are present in the composition in a total amount of about 89.8 wt. % to about 94.8 wt. %; and the high HLB surfactant and the low HLB surfactant are present in the composition in a ratio of greater than 60:40 to about 90:10.

24.-30. (canceled)

31. The pharmaceutical composition of claim 7 consisting of a compound of Formula I, or a stereoisomer or a tautomer thereof, or a pharmaceutically acceptable salt thereof; PEG 40 hydrogenated castor oil; and propylene glycol monocaprylate.

32.-41. (canceled)

42. The pharmaceutical composition of claim 1, wherein the compound is selected from the group consisting of: 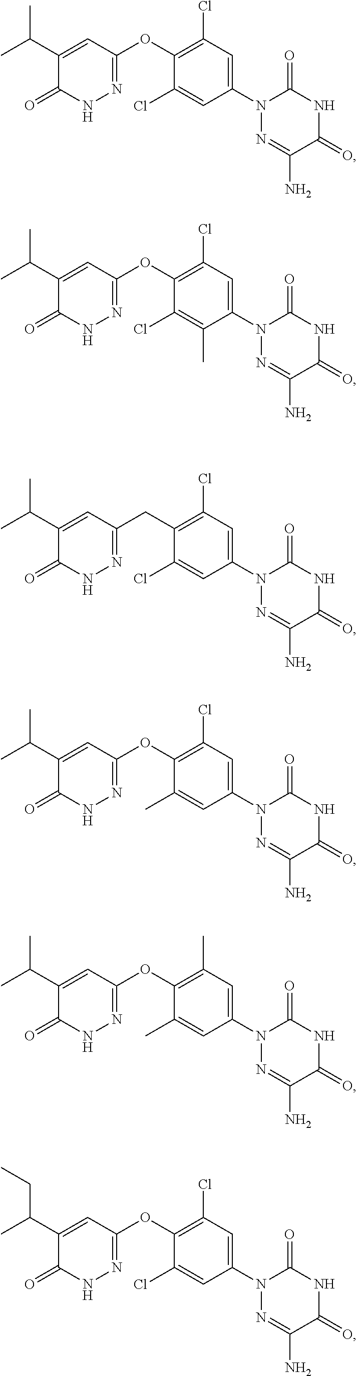 [SMILES: CC(C)c1cc(Oc2c(Cl)cc(-n3nc(N)c(=O)[nH]c3=O)cc2Cl)n[nH]c1=O ; Cc1c(-n2nc(N)c(=O)[nH]c2=O)cc(Cl)c(Oc2cc(C(C)C)c(=O)[nH]n2)c1Cl ; CC(C)c1cc(Cc2c(Cl)cc(-n3nc(N)c(=O)[nH]c3=O)cc2Cl)n[nH]c1=O ; Cc1cc(-n2nc(N)c(=O)[nH]c2=O)cc(Cl)c1Oc1cc(C(C)C)c(=O)[nH]n1 ; Cc1cc(-n2nc(N)c(=O)[nH]c2=O)cc(C)c1Oc1cc(C(C)C)c(=O)[nH]n1 ; CCC(C)c1cc(Oc2c(Cl)cc(-n3nc(N)c(=O)[nH]c3=O)cc2Cl)n[nH]c1=O] 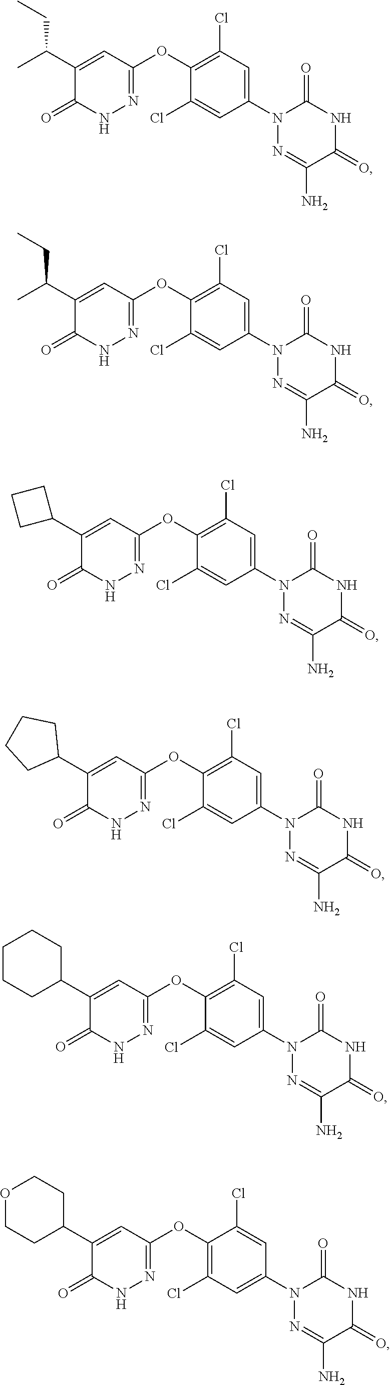 [SMILES: CC[C@@H](C)c1cc(Oc2c(Cl)cc(-n3nc(N)c(=O)[nH]c3=O)cc2Cl)n[nH]c1=O ; CC[C@H](C)c1cc(Oc2c(Cl)cc(-n3nc(N)c(=O)[nH]c3=O)cc2Cl)n[nH]c1=O ; Nc1nn(-c2cc(Cl)c(Oc3cc(C4CCC4)c(=O)[nH]n3)c(Cl)c2)c(=O)[nH]c1=O ; Nc1nn(-c2cc(Cl)c(Oc3cc(C4CCCC4)c(=O)[nH]n3)c(Cl)c2)c(=O)[nH]c1=O ; Nc1nn(-c2cc(Cl)c(Oc3cc(C4CCCCC4)c(=O)[nH]n3)c(Cl)c2)c(=O)[nH]c1=O ; Nc1nn(-c2cc(Cl)c(Oc3cc(C4CCOCC4)c(=O)[nH]n3)c(Cl)c2)c(=O)[nH]c1=O] 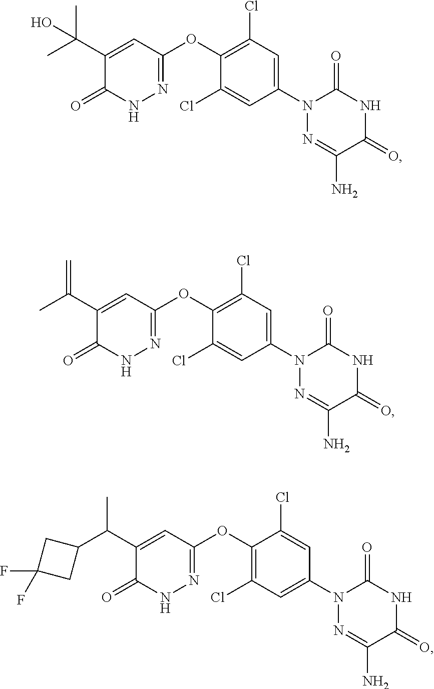 [SMILES: CC(C)(O)c1cc(Oc2c(Cl)cc(-n3nc(N)c(=O)[nH]c3=O)cc2Cl)n[nH]c1=O ; C=C(C)c1cc(Oc2c(Cl)cc(-n3nc(N)c(=O)[nH]c3=O)cc2Cl)n[nH]c1=O ; CC(c1cc(Oc2c(Cl)cc(-n3nc(N)c(=O)[nH]c3=O)cc2Cl)n[nH]c1=O)C1CC(F)(F)C1] 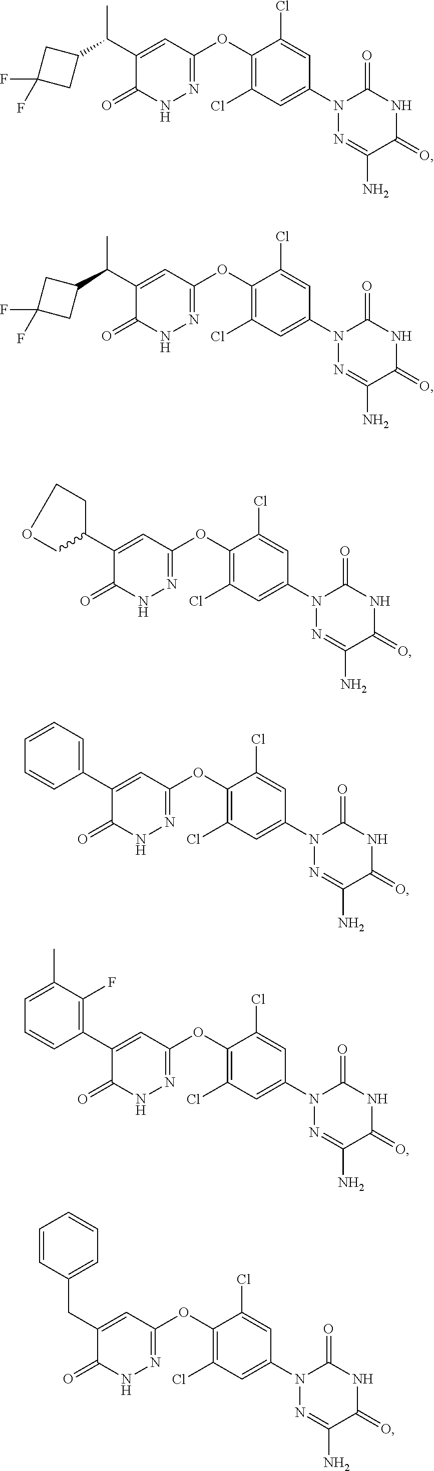 [SMILES: C[C@H](c1cc(Oc2c(Cl)cc(-n3nc(N)c(=O)[nH]c3=O)cc2Cl)n[nH]c1=O)C1CC(F)(F)C1 ; C[C@@H](c1cc(Oc2c(Cl)cc(-n3nc(N)c(=O)[nH]c3=O)cc2Cl)n[nH]c1=O)C1CC(F)(F)C1 ; Nc1nn(-c2cc(Cl)c(Oc3cc(C4CCOC4)c(=O)[nH]n3)c(Cl)c2)c(=O)[nH]c1=O ; Nc1nn(-c2cc(Cl)c(Oc3cc(-c4ccccc4)c(=O)[nH]n3)c(Cl)c2)c(=O)[nH]c1=O ; Cc1cccc(-c2cc(Oc3c(Cl)cc(-n4nc(N)c(=O)[nH]c4=O)cc3Cl)n[nH]c2=O)c1F ; Nc1nn(-c2cc(Cl)c(Oc3cc(Cc4ccccc4)c(=O)[nH]n3)c(Cl)c2)c(=O)[nH]c1=O] 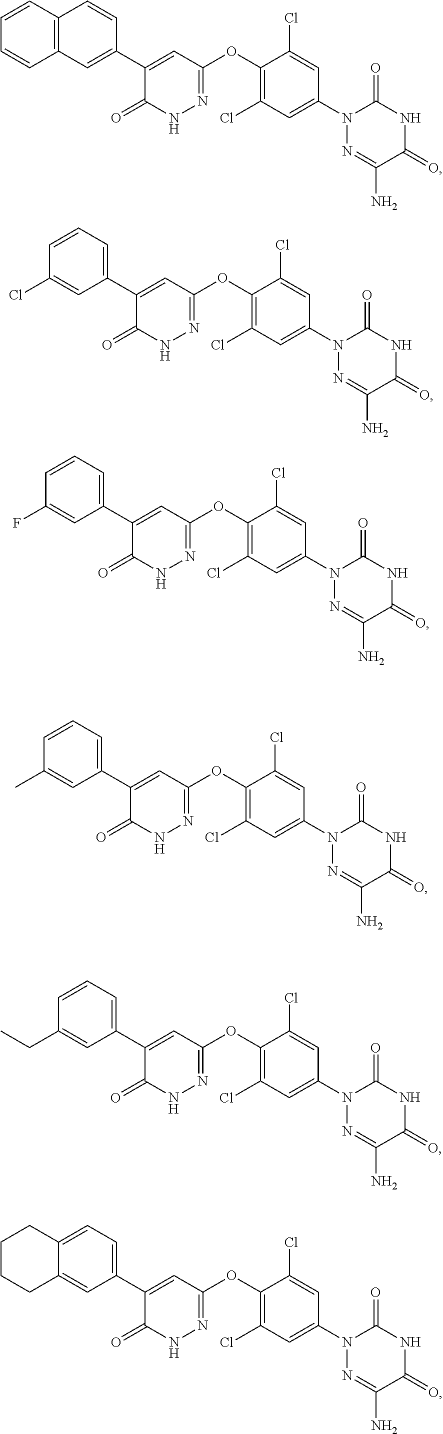 [SMILES: Nc1nn(-c2cc(Cl)c(Oc3cc(-c4ccc5ccccc5c4)c(=O)[nH]n3)c(Cl)c2)c(=O)[nH]c1=O ; Nc1nn(-c2cc(Cl)c(Oc3cc(-c4cccc(Cl)c4)c(=O)[nH]n3)c(Cl)c2)c(=O)[nH]c1=O ; Nc1nn(-c2cc(Cl)c(Oc3cc(-c4cccc(F)c4)c(=O)[nH]n3)c(Cl)c2)c(=O)[nH]c1=O ; Cc1cccc(-c2cc(Oc3c(Cl)cc(-n4nc(N)c(=O)[nH]c4=O)cc3Cl)n[nH]c2=O)c1 ; CCc1cccc(-c2cc(Oc3c(Cl)cc(-n4nc(N)c(=O)[nH]c4=O)cc3Cl)n[nH]c2=O)c1 ; Nc1nn(-c2cc(Cl)c(Oc3cc(-c4ccc5c(c4)CCCC5)c(=O)[nH]n3)c(Cl)c2)c(=O)[nH]c1=O] 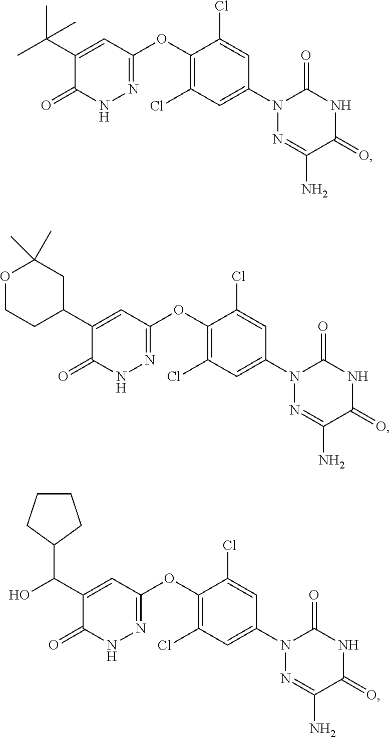 [SMILES: CC(C)(C)c1cc(Oc2c(Cl)cc(-n3nc(N)c(=O)[nH]c3=O)cc2Cl)n[nH]c1=O ; CC1(C)CC(c2cc(Oc3c(Cl)cc(-n4nc(N)c(=O)[nH]c4=O)cc3Cl)n[nH]c2=O)CCO1 ; Nc1nn(-c2cc(Cl)c(Oc3cc(C(O)C4CCCC4)c(=O)[nH]n3)c(Cl)c2)c(=O)[nH]c1=O] 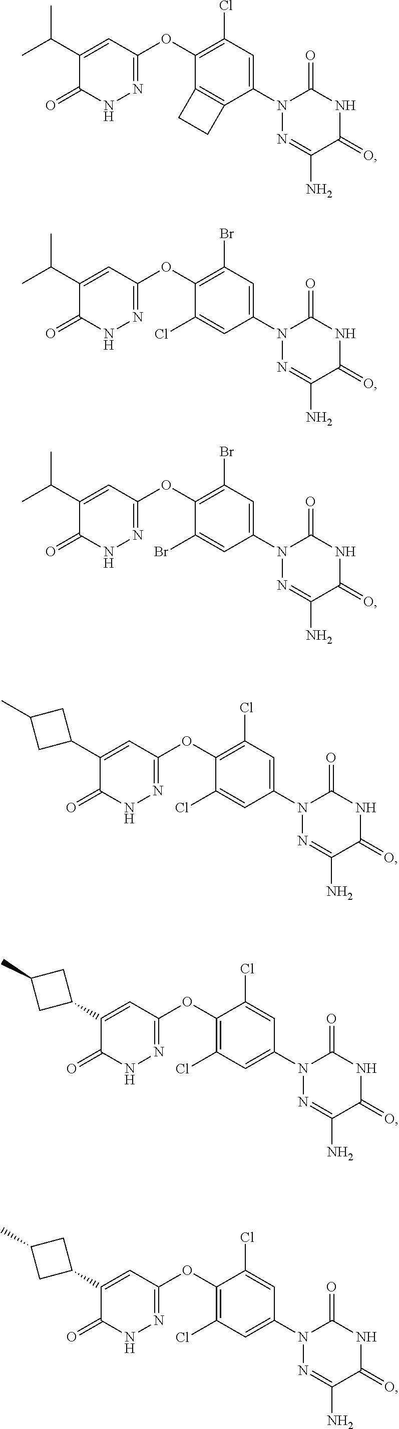 [SMILES: CC(C)c1cc(Oc2c(Cl)cc(-n3nc(N)c(=O)[nH]c3=O)c3c2CC3)n[nH]c1=O ; CC(C)c1cc(Oc2c(Cl)cc(-n3nc(N)c(=O)[nH]c3=O)cc2Br)n[nH]c1=O ; CC(C)c1cc(Oc2c(Br)cc(-n3nc(N)c(=O)[nH]c3=O)cc2Br)n[nH]c1=O ; CC1CC(c2cc(Oc3c(Cl)cc(-n4nc(N)c(=O)[nH]c4=O)cc3Cl)n[nH]c2=O)C1 ; C[C@H]1C[C@H](c2cc(Oc3c(Cl)cc(-n4nc(N)c(=O)[nH]c4=O)cc3Cl)n[nH]c2=O)C1 ; C[C@H]1C[C@@H](c2cc(Oc3c(Cl)cc(-n4nc(N)c(=O)[nH]c4=O)cc3Cl)n[nH]c2=O)C1] 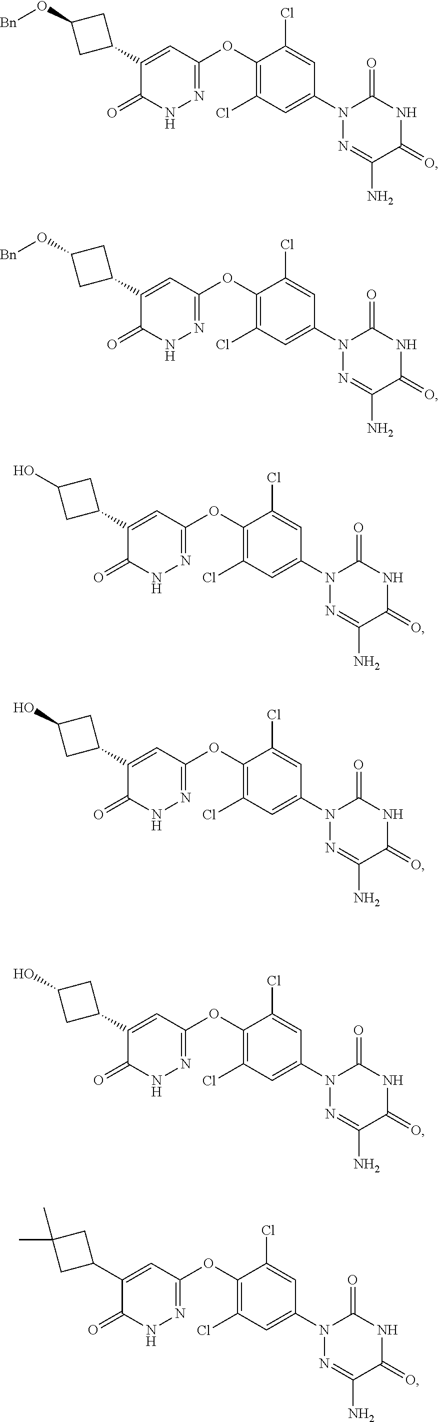 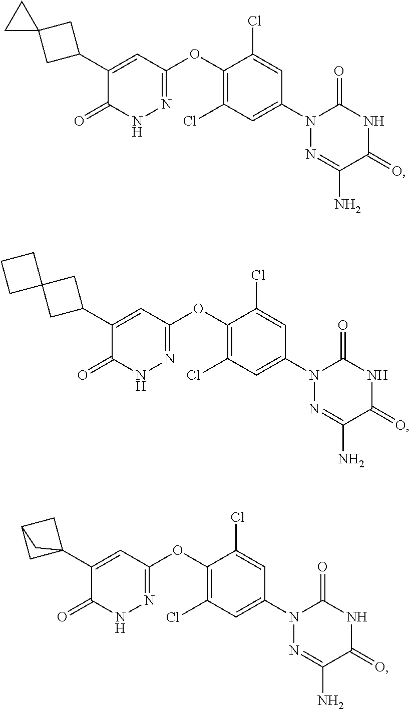 [SMILES: Nc1nn(-c2cc(Cl)c(Oc3cc(C4CC5(CC5)C4)c(=O)[nH]n3)c(Cl)c2)c(=O)[nH]c1=O ; Nc1nn(-c2cc(Cl)c(Oc3cc(C4CC5(CCC5)C4)c(=O)[nH]n3)c(Cl)c2)c(=O)[nH]c1=O ; Nc1nn(-c2cc(Cl)c(Oc3cc(C45CC(C4)C5)c(=O)[nH]n3)c(Cl)c2)c(=O)[nH]c1=O] 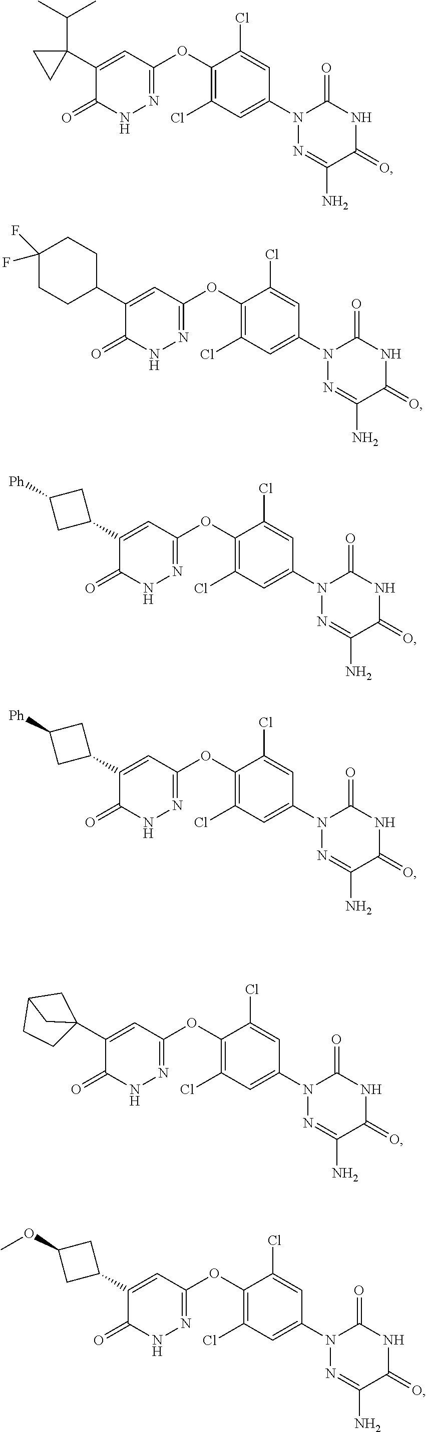 [SMILES: CC(C)C1(c2cc(Oc3c(Cl)cc(-n4nc(N)c(=O)[nH]c4=O)cc3Cl)n[nH]c2=O)CC1 ; Nc1nn(-c2cc(Cl)c(Oc3cc(C4CCC(F)(F)CC4)c(=O)[nH]n3)c(Cl)c2)c(=O)[nH]c1=O ; Nc1nn(-c2cc(Cl)c(Oc3cc([C@H]4C[C@@H](c5ccccc5)C4)c(=O)[nH]n3)c(Cl)c2)c(=O)[nH]c1=O ; Nc1nn(-c2cc(Cl)c(Oc3cc([C@H]4C[C@H](c5ccccc5)C4)c(=O)[nH]n3)c(Cl)c2)c(=O)[nH]c1=O ; Nc1nn(-c2cc(Cl)c(Oc3cc(C45CCC(C4)C5)c(=O)[nH]n3)c(Cl)c2)c(=O)[nH]c1=O ; CO[C@H]1C[C@H](c2cc(Oc3c(Cl)cc(-n4nc(N)c(=O)[nH]c4=O)cc3Cl)n[nH]c2=O)C1] 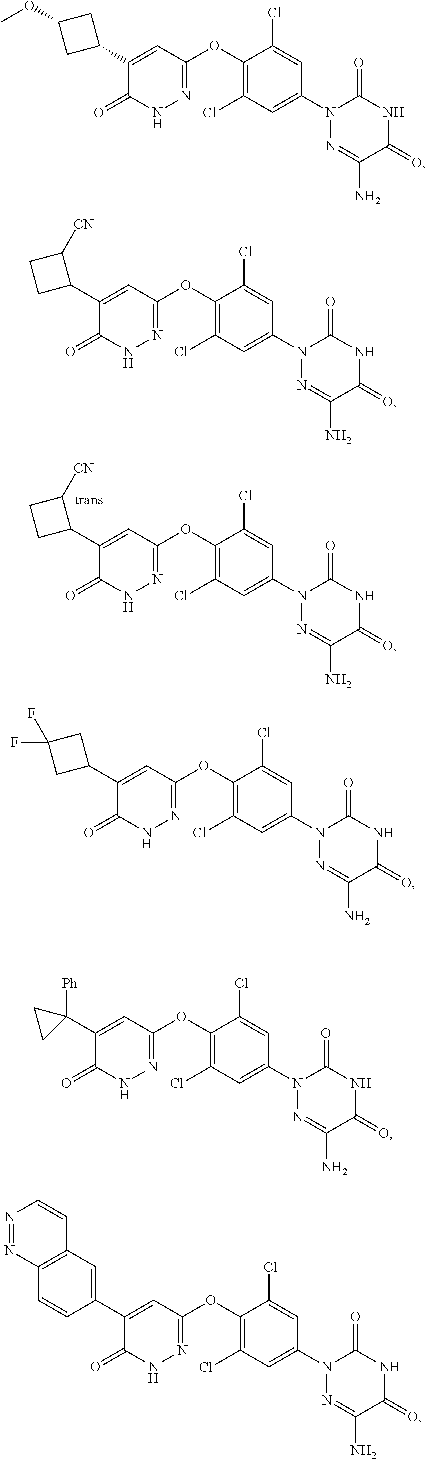 [SMILES: CO[C@H]1C[C@@H](c2cc(Oc3c(Cl)cc(-n4nc(N)c(=O)[nH]c4=O)cc3Cl)n[nH]c2=O)C1 ; N#CC1CCC1c1cc(Oc2c(Cl)cc(-n3nc(N)c(=O)[nH]c3=O)cc2Cl)n[nH]c1=O ; N#CC1CCC1c1cc(Oc2c(Cl)cc(-n3nc(N)c(=O)[nH]c3=O)cc2Cl)n[nH]c1=O ; Nc1nn(-c2cc(Cl)c(Oc3cc(C4CC(F)(F)C4)c(=O)[nH]n3)c(Cl)c2)c(=O)[nH]c1=O ; Nc1nn(-c2cc(Cl)c(Oc3cc(C4(c5ccccc5)CC4)c(=O)[nH]n3)c(Cl)c2)c(=O)[nH]c1=O ; Nc1nn(-c2cc(Cl)c(Oc3cc(-c4ccc5nnccc5c4)c(=O)[nH]n3)c(Cl)c2)c(=O)[nH]c1=O] 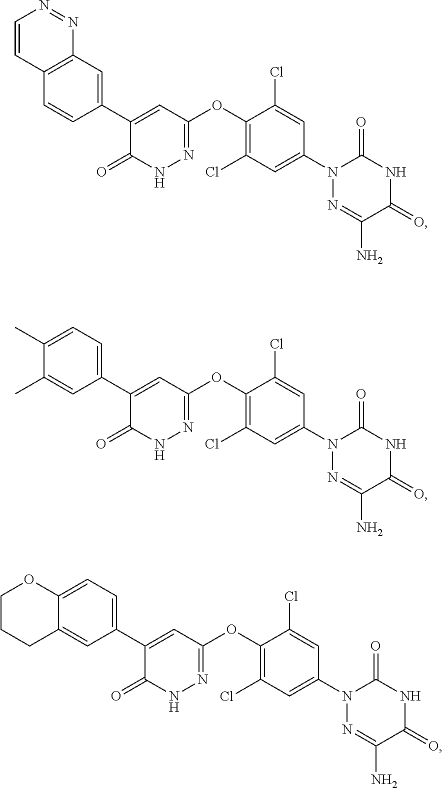 [SMILES: Nc1nn(-c2cc(Cl)c(Oc3cc(-c4ccc5ccnnc5c4)c(=O)[nH]n3)c(Cl)c2)c(=O)[nH]c1=O ; Cc1ccc(-c2cc(Oc3c(Cl)cc(-n4nc(N)c(=O)[nH]c4=O)cc3Cl)n[nH]c2=O)cc1C ; Nc1nn(-c2cc(Cl)c(Oc3cc(-c4ccc5c(c4)CCCO5)c(=O)[nH]n3)c(Cl)c2)c(=O)[nH]c1=O] 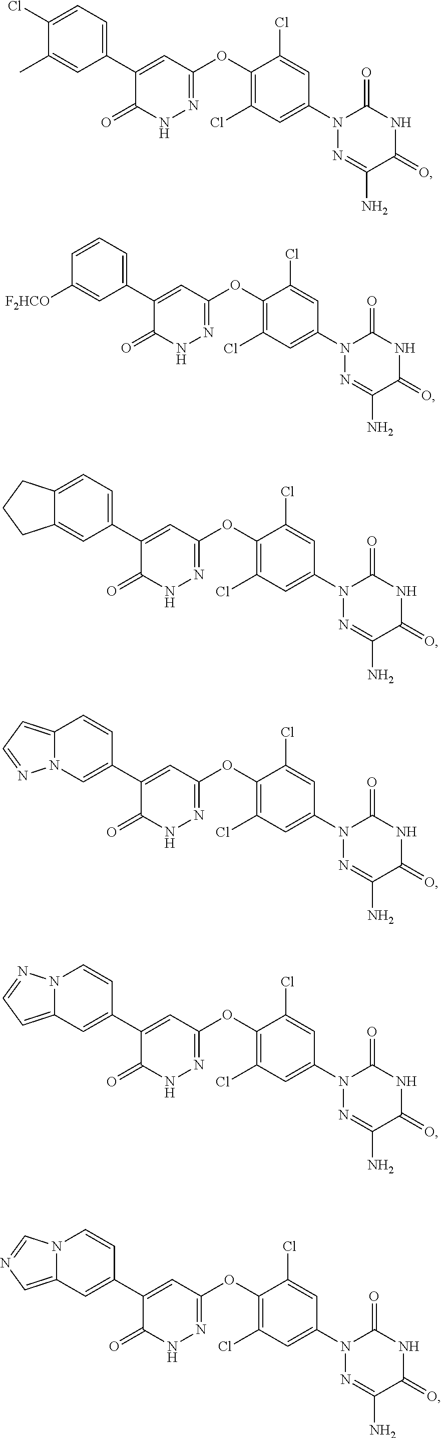 [SMILES: Cc1cc(-c2cc(Oc3c(Cl)cc(-n4nc(N)c(=O)[nH]c4=O)cc3Cl)n[nH]c2=O)ccc1Cl ; Nc1nn(-c2cc(Cl)c(Oc3cc(-c4cccc(OC(F)F)c4)c(=O)[nH]n3)c(Cl)c2)c(=O)[nH]c1=O ; Nc1nn(-c2cc(Cl)c(Oc3cc(-c4ccc5c(c4)CCC5)c(=O)[nH]n3)c(Cl)c2)c(=O)[nH]c1=O ; Nc1nn(-c2cc(Cl)c(Oc3cc(-c4ccc5ccnn5c4)c(=O)[nH]n3)c(Cl)c2)c(=O)[nH]c1=O ; Nc1nn(-c2cc(Cl)c(Oc3cc(-c4ccn5nccc5c4)c(=O)[nH]n3)c(Cl)c2)c(=O)[nH]c1=O ; Nc1nn(-c2cc(Cl)c(Oc3cc(-c4ccn5cncc5c4)c(=O)[nH]n3)c(Cl)c2)c(=O)[nH]c1=O] 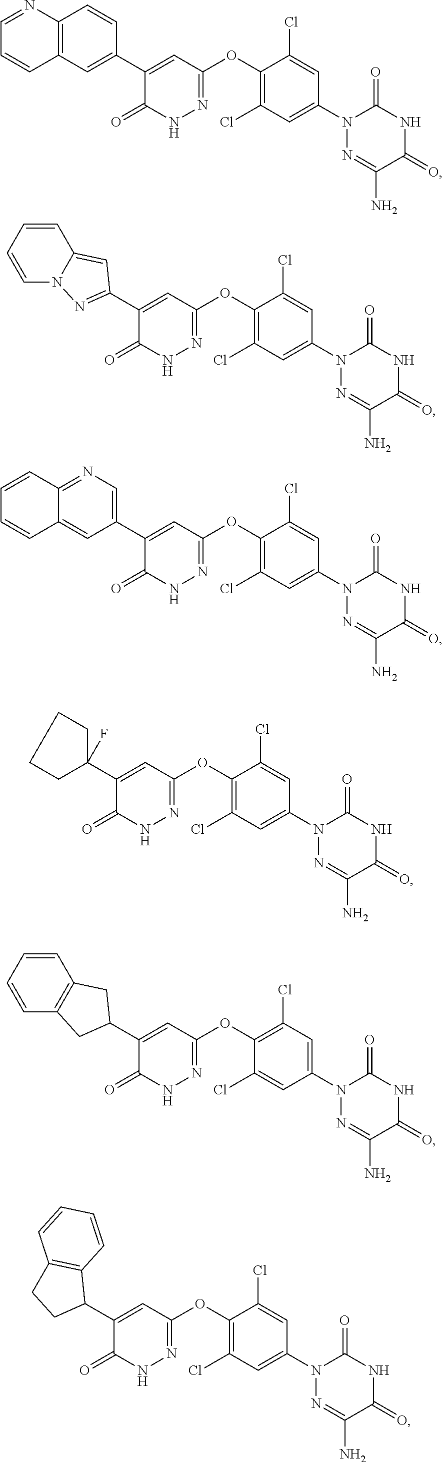 [SMILES: Nc1nn(-c2cc(Cl)c(Oc3cc(-c4ccc5ncccc5c4)c(=O)[nH]n3)c(Cl)c2)c(=O)[nH]c1=O ; Nc1nn(-c2cc(Cl)c(Oc3cc(-c4cc5ccccn5n4)c(=O)[nH]n3)c(Cl)c2)c(=O)[nH]c1=O ; Nc1nn(-c2cc(Cl)c(Oc3cc(-c4cnc5ccccc5c4)c(=O)[nH]n3)c(Cl)c2)c(=O)[nH]c1=O ; Nc1nn(-c2cc(Cl)c(Oc3cc(C4(F)CCCC4)c(=O)[nH]n3)c(Cl)c2)c(=O)[nH]c1=O ; Nc1nn(-c2cc(Cl)c(Oc3cc(C4Cc5ccccc5C4)c(=O)[nH]n3)c(Cl)c2)c(=O)[nH]c1=O ; Nc1nn(-c2cc(Cl)c(Oc3cc(C4CCc5ccccc54)c(=O)[nH]n3)c(Cl)c2)c(=O)[nH]c1=O] 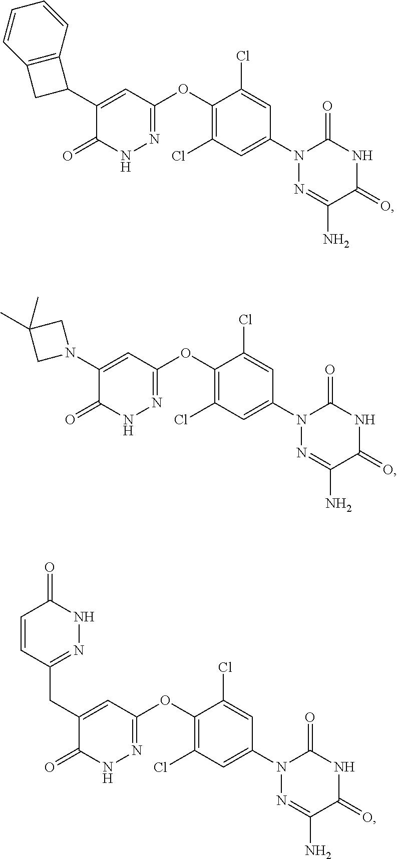 [SMILES: Nc1nn(-c2cc(Cl)c(Oc3cc(C4Cc5ccccc54)c(=O)[nH]n3)c(Cl)c2)c(=O)[nH]c1=O ; CC1(C)CN(c2cc(Oc3c(Cl)cc(-n4nc(N)c(=O)[nH]c4=O)cc3Cl)n[nH]c2=O)C1 ; Nc1nn(-c2cc(Cl)c(Oc3cc(Cc4ccc(=O)[nH]n4)c(=O)[nH]n3)c(Cl)c2)c(=O)[nH]c1=O] 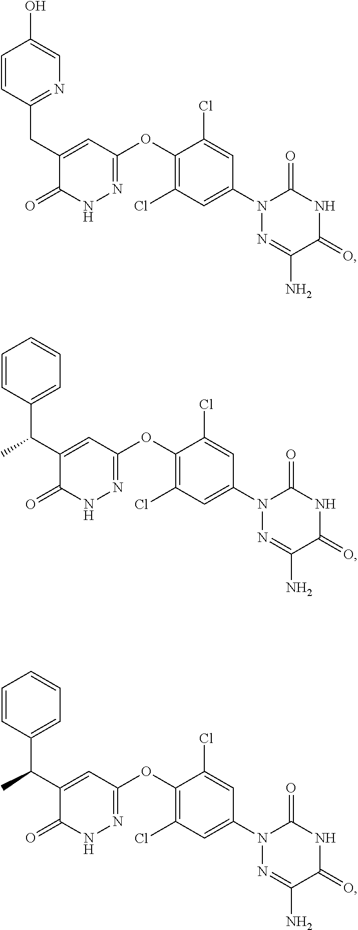 [SMILES: Nc1nn(-c2cc(Cl)c(Oc3cc(Cc4ccc(O)cn4)c(=O)[nH]n3)c(Cl)c2)c(=O)[nH]c1=O ; C[C@H](c1ccccc1)c1cc(Oc2c(Cl)cc(-n3nc(N)c(=O)[nH]c3=O)cc2Cl)n[nH]c1=O ; C[C@@H](c1ccccc1)c1cc(Oc2c(Cl)cc(-n3nc(N)c(=O)[nH]c3=O)cc2Cl)n[nH]c1=O] 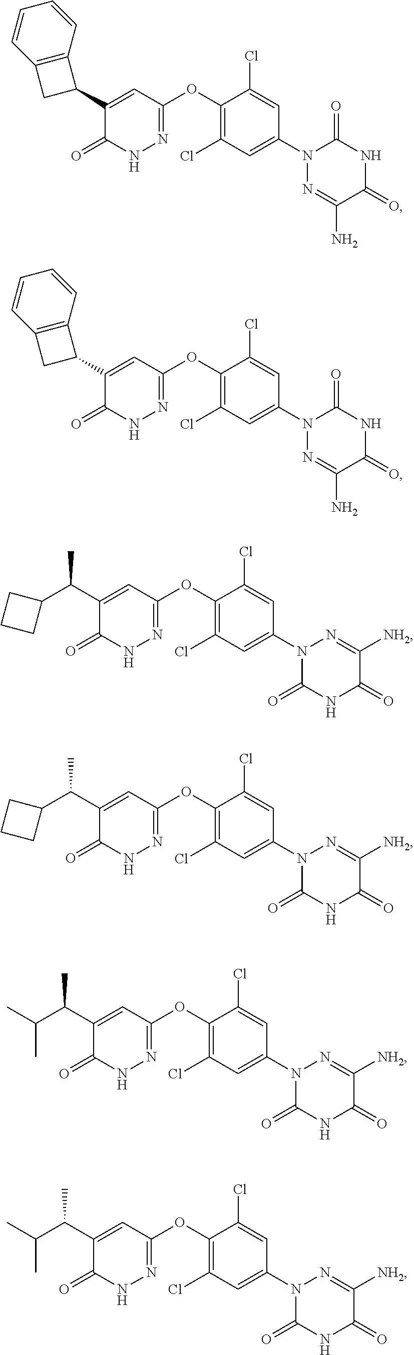 [SMILES: Nc1nn(-c2cc(Cl)c(Oc3cc([C@H]4Cc5ccccc54)c(=O)[nH]n3)c(Cl)c2)c(=O)[nH]c1=O ; Nc1nn(-c2cc(Cl)c(Oc3cc([C@@H]4Cc5ccccc54)c(=O)[nH]n3)c(Cl)c2)c(=O)[nH]c1=O ; C[C@@H](c1cc(Oc2c(Cl)cc(-n3nc(N)c(=O)[nH]c3=O)cc2Cl)n[nH]c1=O)C1CCC1 ; C[C@H](c1cc(Oc2c(Cl)cc(-n3nc(N)c(=O)[nH]c3=O)cc2Cl)n[nH]c1=O)C1CCC1 ; CC(C)[C@@H](C)c1cc(Oc2c(Cl)cc(-n3nc(N)c(=O)[nH]c3=O)cc2Cl)n[nH]c1=O ; CC(C)[C@H](C)c1cc(Oc2c(Cl)cc(-n3nc(N)c(=O)[nH]c3=O)cc2Cl)n[nH]c1=O] 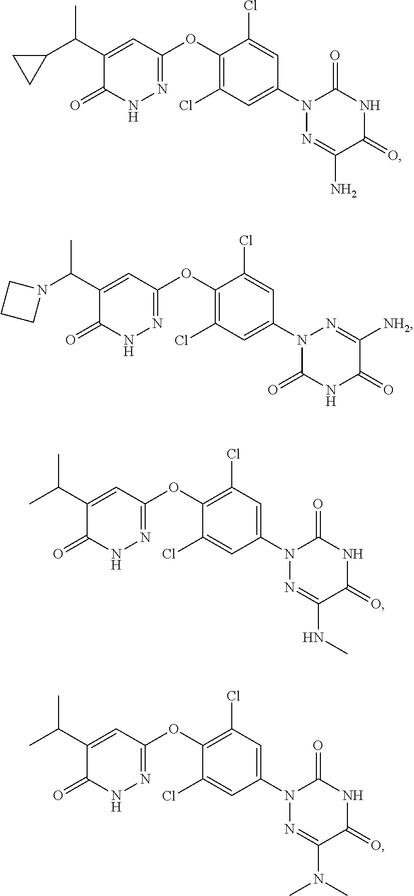 [SMILES: CC(c1cc(Oc2c(Cl)cc(-n3nc(N)c(=O)[nH]c3=O)cc2Cl)n[nH]c1=O)C1CC1 ; CC(c1cc(Oc2c(Cl)cc(-n3nc(N)c(=O)[nH]c3=O)cc2Cl)n[nH]c1=O)N1CCC1 ; CNc1nn(-c2cc(Cl)c(Oc3cc(C(C)C)c(=O)[nH]n3)c(Cl)c2)c(=O)[nH]c1=O ; CC(C)c1cc(Oc2c(Cl)cc(-n3nc(N(C)C)c(=O)[nH]c3=O)cc2Cl)n[nH]c1=O] 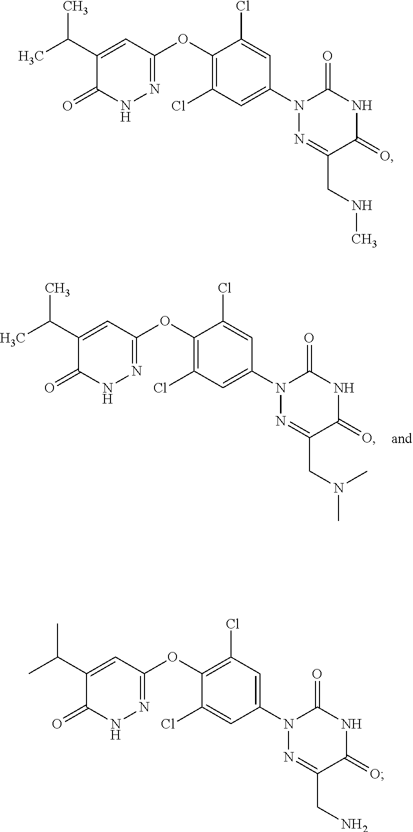
or a stereoisomer or a tautomer thereof, or a pharmaceutically acceptable salt thereof.

43. (canceled)

44. The pharmaceutical composition of claim 31 consisting of: 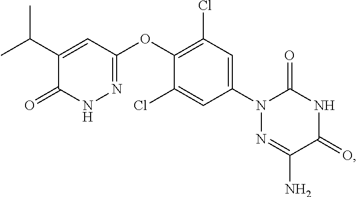 [SMILES: CC(C)c1cc(Oc2c(Cl)cc(-n3nc(N)c(=O)[nH]c3=O)cc2Cl)n[nH]c1=O]
or a pharmaceutically acceptable salt thereof, in an amount of from about 0.1 wt. % to about 0.3 wt. %; and
propylene glycol monocaprylate and PEG 40 hydrogenated castor oil in a ratio of from about 30:70 to about 20:80 (propylene glycol monocaprylate:PEG 40 hydrogenated castor oil).

45. (canceled)

46. The pharmaceutical composition of claim 44, wherein 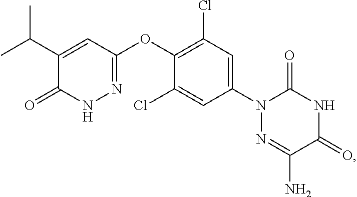 [SMILES: CC(C)c1cc(Oc2c(Cl)cc(-n3nc(N)c(=O)[nH]c3=O)cc2Cl)n[nH]c1=O]
or the pharmaceutically acceptable salt thereof, is present in an amount of from about 100 μg to about 2000 μg.

47. (canceled)

48. The pharmaceutical composition of claim 1, wherein the composition is in the form of a capsule.

49. The pharmaceutical composition of claim 48, wherein the capsule is a soft gel capsule.

50. A method of treating a disorder or disease in a subject in need thereof, the method comprising administering to the subject a therapeutically effective amount of the pharmaceutical composition of claim 1, wherein the disorder or disease is selected from (metabolic dysfunction-associated steatohepatitis (MASH), obesity, hyperlipidemia, hypercholesterolemia, diabetes, liver steatosis, atherosclerosis, cardiovascular diseases, hypothyroidism, and thyroid cancer.

51. The method of claim 50, wherein the pharmaceutical composition is administered in combination with a KHK inhibitor, an FXR agonist, a SSAO inhibitor, a FASN inhibitor, or a SCD1 modulator.

52.-55. (canceled)

56. A soft gel capsule comprising a fill formulation and a capsule shell, wherein:
the fill formulation comprises a compound of Formula I, or a stereoisomer or a tautomer thereof, or a pharmaceutically acceptable salt thereof, in an amount of from about 0.1 wt. % to about 0.3 wt. % of the fill formulation; and propylene glycol monocaprylate and PEG 40 hydrogenated castor oil in a ratio of from about 30:70 to about 20:80 (propylene glycol monocaprylate:PEG 40 hydrogenated castor oil); and
the capsule shell is prepared from an initial composition comprising gelatin, glycerol, dye, titanium dioxide, and purified water; and the initial composition undergoes drying to substantially remove the purified water to prepare the capsule shell.

57. (canceled)

58. (canceled)

59. A soft gel capsule comprising a fill formulation and a capsule shell, wherein:
the fill formulation comprises a compound of Formula I, or a stereoisomer or a tautomer thereof, or a pharmaceutically acceptable salt thereof, in an amount of from about 0.025 wt. % to about 1.0 wt. % of the fill formulation; and PEG 40 hydrogenated castor oil and propylene glycol monocaprylate in a ratio of from about 50:50 to about 90:10 (PEG 40 hydrogenated castor oil:propylene glycol monocaprylate); and
the capsule shell is prepared from an initial composition comprising gelatin, a mixture of sorbitol and sorbitan, glycerin, titanium dioxide, and purified water; and the initial composition undergoes drying to substantially remove the purified water to prepare the capsule shell.

60.-66. (canceled)

67. A method to prepare a soft gel capsule, wherein the method comprises:
combining (i) a compound of Formula I, or a stereoisomer or a tautomer thereof, or a pharmaceutically acceptable salt thereof; (ii) propylene glycol monocaprylate; and (iii) PEG 40 hydrogenated castor oil under oxygen-free conditions to form a fill formulation;
combining gelatin, a mixture of sorbitol and sorbitan, glycerin, and titanium dioxide in purified water to provide a gelatin mass; and
encapsulating the fill formulation within the gelatin mass followed by drying to form individual soft gel capsules.

68.-76. (canceled)
